SEQUENCE LISTING (29 sequences)

SEQ ID NO: 1 - protein, 15 aa, Plasmodium berghei
MVRIKPGSANKPSDD

SEQ ID NO: 2 - protein, 15 aa, Plasmodium berghei
MVRIKYGSYNNKPSD

SEQ ID NO: 3 - protein, 13 aa, Plasmodium berghei
MVRIKPGSANKPS

SEQ ID NO: 4 - protein, 9 aa, Plasmodium berghei
MVRIKPGSA

SEQ ID NO: 5 - protein, 15 aa, Artificial Sequence
  synthetic construct; scrambled Circumsporozoite protein
GRVDPSNKSMPKDIA

SEQ ID NO: 6 - protein, 10 aa, Homo sapiens
SQAVQEHAST

SEQ ID NO: 7 - protein, 13 aa, Homo sapiens
SQAVQEHASTNMG

SEQ ID NO: 8 - protein, 13 aa, Artificial Sequence
  synthetic construct; scrambled region of human retinoid and thyroid hormone receptor SMRT
EVAQHSQASTMNG

SEQ ID NO: 9 - protein, 14 aa, Artificial Sequence
  synthetic construct; scrambled PIF peptide
GQASSAQMNSTGVH

SEQ ID NO: 10 - protein, 18 aa, Artificial Sequence
  synthetic construct; PIF peptide
SGIVIYQYMDDRYVGSDL

SEQ ID NO: 11 - protein, 11 aa, Artificial Sequence
  synthetic construct; scrambled region of Circumsporozoite protein
GMRELQRSANK

SEQ ID NO: 12 - protein, 9 aa, Artificial Sequence
  synthetic construct; PIF peptide
VIIIAQYMD

SEQ ID NO: 13 - protein, 15 aa, Artificial Sequence
  synthetic construct; PIF peptide
MVRIKPGSANKPSDD

SEQ ID NO: 14 - protein, 13 aa, Artificial Sequence
  synthetic construct; PIF peptide
SQAVQEHASTNMG

SEQ ID NO: 15 - protein, 18 aa, Artificial Sequence
  synthetic construct; PIF peptide
SGIVIYQYMDDRYVGSDL

SEQ ID NO: 16 - protein, 9 aa, Artificial Sequence
  synthetic construct; PIF peptide
MVRIKPGSA

SEQ ID NO: 17 - protein, 9 aa, Artificial Sequence
  synthetic construct; PIF peptide
VIIIAQYMD

SEQ ID NO: 18 - protein, 5 aa, Artificial Sequence
  synthetic construct; PIF peptide
MVRIK

SEQ ID NO: 19 - protein, 4 aa, Artificial Sequence
  synthetic construct; PIF peptide
PGSA

SEQ ID NO: 20 - protein, 15 aa, Artificial Sequence
  synthetic construct; PIF peptide
  misc_feature (3)..(3): Xaa can be any naturally occurring amino acid
MVXIKPGSANKPSDD

SEQ ID NO: 21 - protein, 15 aa, Artificial Sequence
  synthetic construct; PIF peptide
  misc_feature (1)..(1): Xaa can be any naturally occurring amino acid
XVRIKPGSANKPSDD

SEQ ID NO: 22 - protein, 15 aa, Artificial Sequence
  synthetic construct; PIF peptide
  misc_feature (1)..(1): Xaa can be any naturally occurring amino acid
  misc_feature (3)..(3): Xaa can be any naturally occurring amino acid
XVXIKPGSANKPSDD

SEQ ID NO: 23 - protein, 15 aa, Artificial Sequence
  synthetic construct; PIF peptide
  misc_feature (6)..(6): Xaa can be any naturally occurring amino acid
MVRIKXGSANKPSDD

SEQ ID NO: 24 - protein, 15 aa, Artificial Sequence
  synthetic construct; PIF peptide
  misc_feature (4)..(4): Xaa can be any naturally occurring amino acid
MVRXKPGSANKPSDD

SEQ ID NO: 25 - protein, 15 aa, Artificial Sequence
  synthetic construct; PIF peptide
  misc_feature (2)..(2): Xaa can be any naturally occurring amino acid
MXRIKPGSANKPSDD

SEQ ID NO: 26 - protein, 15 aa, Artificial Sequence
  synthetic construct; PIF peptide
MVRIKEGSANKPSDD

SEQ ID NO: 27 - protein, 15 aa, Artificial Sequence
  synthetic construct; PIF peptide
MVRGKPGSANKPSDD

SEQ ID NO: 28 - protein, 15 aa, Artificial Sequence
  synthetic construct; PIF peptide
MERIKPGSANKPSDD

SEQ ID NO: 29 - protein, 15 aa, Artificial Sequence
  synthetic construct; PIF peptide
AVRIKPGSANKPSDD

(19) United States
(12) Patent Application Publication
(10) Pub. No.: US 2023/0190867 A1
(43) Pub. Date: Jun. 22, 2023

(54) METHODS OF TREATING NASH AND COMPOSITIONS THEREFORE

(71) Applicant: BIOINCEPT, LLC, Westport, CT (US)

(72) Inventor: Eytan R. Barnea, New York, NY (US)

(21) Appl. No.: 16/975,375

(22) PCT Filed: Feb. 22, 2019
(86) PCT No.: PCT/US2019/019256

§ 371 Date: Aug. 24, 2020

Related U.S. Application Data
(60) Provisional application No. 62/634,189, filed on Feb. 22, 2018.

(51) Int. Cl. A61K 38/17; A61P 31/22; A61P 1/16; A61K 45/06

(52) U.S. Cl. CPC A61K 38/1709; A61P 31/22; A61P 1/16; A61K 45/06

(57) ABSTRACT

This application describes compounds that are preimplantation factor (PIF) peptides, mimetics thereof, pharmaceutically acceptable salts thereof, or combinations thereof. This application also describes the use of those compounds for treatment and prevention of non-alcoholic steatohepatitis (NASH), liver fibrosis and non-alcoholic fatty liver disease (NAFLD).

### CROSS REFERENCE TO RELATED APPLICATIONS

The present application is a national stage application filed under 35 U.S.C. § 371 of International Application No. PCT/US2019/019256, filed Feb. 22, 2019, which claims priority to U.S. Provisional Application No. 62/634,189, filed Feb. 22, 2018, the entire contents of each of the aforementioned applications are incorporated by reference in their entireties.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is 37928_0030U2_Sequence_Listing. The size of the text file is 8 KB and the text file was created on Aug. 24, 2020

### FIELD OF INVENTION

The disclosure relates to methods of treating or preventing non-alcoholic fatty liver disease in a subject in need thereof.

### BACKGROUND

Non-alcoholic fatty liver disease, (NAFLD), is among the most common liver diseases in the world. NAFLD encompasses a broad spectrum of pathological conditions ranging from simple steatosis to steatohepatitis, fibrosis and finally even cirrhosis. However, only a minority of patients progress to end-stage disease. In 10 years, only 3% will become cirrhotic (Giridahar 2013). There is rising concern that NAFLD leads to development of cancer, specifically hepatocellular carcinoma (HCC). Generally, the liver cells store very little amount of fat. However, in the case of NAFLD diseases, there is fat accumulation in the liver that is damaging to some patients, and yet causes no symptoms to others. Fibrosis is a major driver in patients that have advanced NAFLD. There are several clinical similarities among patients with NAFLD. These patients frequently display insulin resistance that leads to diabetes, being overweight, increased visceral fat, hyperlipidemia and high blood pressure. These patients also have increased rate of mortality as compared to the general population when paired by age and sex (Duran 2016).

Nonalcoholic steatohepatitis (NASH), a severe form of NAFLD, is characterized by liver inflammation and damage, caused by a buildup of fat in the liver. NAFLD affects about 25-30% of the general population, and about a quarter of these patients have non-alcoholic steatohepatitis (NASH). NASH is considered a more severe form of NAFLD because it can cause scar tissue to build up that may lead to cirrhosis and liver failure or even hepatocellular cancer that requires liver transplantation.

At present, NASH is becoming a leading cause of hepatic damage that results in liver transplantation. Liver transplantation is the only option once advanced cirrhosis develops, but obesity and the complications of diabetes may preclude consideration for liver transplantation.

Risk factors for NASH include obesity, diabetes, hypertension and hyperlipidemia, viral hepatitis, especially hepatitis C, and exposure to toxins. Diagnosis is made by blood tests with liver imaging and ultimately a liver biopsy is required for a definitive diagnosis. Currently, there is no therapy available. Previous therapies have focused on treating the cause, aiming to stop or slow further scarring of the liver, which is sometimes shown to be beneficial. These treatments include: 1. antiviral drugs to eliminate the virus in cases of chronic viral hepatitis; 2. abstinence from alcohol consumption; 3. removal of heavy metals in cases of iron overload (hemochromatosis) or Wilson disease (which causes copper to accumulate); 4. discontinuing drugs that are known to cause fibrosis and limiting exposure to environmental toxins; 5. surgical treatments to remove or dissolve bile duct blockage; and 6. encouraging weight loss, which in turn controls glycemia and lipid levels. The use of alternative treatment by herbals, antioxidants, Vitamin D, and probiotics have also been studied and recommended to patients in a disease where no other promising treatment is available.

### SUMMARY OF EMBODIMENTS

The disclosure provides a method of treating non-alcoholic fatty liver disease (NAFLD) in a subject. In one aspect, the method comprises administering to the subject a pharmaceutical composition comprising: (i) a therapeutically effective amount of Preimplantation Factor (PIF) peptide, compositions thereof, mimetics thereof, pharmaceutically acceptable salts thereof, or combinations of any of the foregoing; and (ii) one or plurality of pharmaceutically acceptable carriers.

The disclosure also provides a method of preventing non-alcoholic fatty liver disease (NAFLD) in a subject. In one aspect, the method comprises administering to the subject a pharmaceutical composition comprising: (i) a therapeutically effective amount of PIF peptide, compositions thereof, mimetics thereof, pharmaceutically acceptable salts thereof, or combinations of any of the foregoing; and (ii) one or plurality of pharmaceutically acceptable carriers.

The disclosure also provides a method of treating non-alcoholic steatohepatitis (NASH) in a subject. In one aspect, the method comprises administering to the subject a pharmaceutical composition comprising: (i) a therapeutically effective amount of PIF peptide, compositions thereof, mimetics thereof, pharmaceutically acceptable salts thereof, or combinations of any of the foregoing; and (ii) one or plurality of pharmaceutically acceptable carriers.

The disclosure also provides a method of preventing non-alcoholic steatohepatitis (NASH) in a subject. In one aspect, the method comprises administering to the subject a pharmaceutical composition comprising: (i) a therapeutically effective amount of PIF peptide, compositions thereof, mimetics thereof, pharmaceutically acceptable salts thereof, or combinations of any of the foregoing; and (ii) one or plurality of pharmaceutically acceptable carriers.

In some embodiments, the therapeutically effective amount is from about 0.01 μg per milliliter of volume to about 10 mg per milliliter of volume. In some embodiments, the therapeutically effective amount is from about 0.1 μg per milliliter of volume to about 5 μg per milliliter of volume.

In some embodiments, the PIF peptide comprises MVRIK (SEQ ID NO: 18) or a pharmaceutically acceptable salt thereof. In some embodiments, the PIF peptide comprises MVRIKPGSANKPSDD (SEQ ID NO: 1) or a pharmaceutically acceptable salt thereof. In some embodiments, the PIF peptide comprises an amino acid sequence that is at least 70% homologous to MVRIKPGSANKPSDD (SEQ ID NO: 1) or a pharmaceutically acceptable salt thereof. In some embodiments, the PIF peptide consists of MVRIKPGSANKPSDD (SEQ ID NO: 1) or a pharmaceutically acceptable salt thereof.

In some embodiments, the methods provided herein may further comprise administering a therapeutically effective amount of an anti-inflammatory compound at least one hour prior to, simultaneously with, or subsequent to administering the pharmaceutical composition. In some embodiments, the anti-inflammatory compound is free of a steroid.

In some embodiments, the methods provided herein may further comprise a step of administering a therapeutically effective amount of PIF peptide, compositions thereof, mimetics thereof, pharmaceutically acceptable salts thereof, or combinations thereof, once, twice, or three times in a 30 day period. In some embodiments, the therapeutically effective amount of PIF peptide is the only therapeutic compound administered to the subject. In some embodiments, the pharmaceutical composition is administered intravenously, intramuscularly, topically intradermally, transmucosally, subcutaneously, sublingually, orally, intravaginally, intraocularly, intranasally, intrarectally, gastrointesinally, intraductally, inthecall, subdurally, exradurally, intraventricularly, intraarticuarly, intraperitoneally, or into the pleural cavity.

### BRIEF DESCRIPTION OF DRAWINGS

FIG. 1 shows a flow diagram for patient disposition in the single ascending dose clinical trial in patients with chronic autoimmune hepatitis (AIH) and compensated chronic liver disease.

FIG. 2 is a graph showing pharmacokinetics of sPIF in the single ascending dose study. Lines represent time course of individual patients and mean serum levels following a single subcutaneous dose of sPIF.

FIG. 3 shows a flow diagram for patient disposition in the multiple ascending dose clinical trial in patients with chronic autoimmune hepatitis (AIH) and compensated chronic liver disease.

FIG. 4A-FIG. 4D are graphs showing the effect of TxpIC 500 ng/ml and sPIF on HepaRg primary cell line. A dose dependent decrease was observed in CXCL10 (FIG. 4A), IL28 (FIG. 4B), CCL5 (FIG. 4C), and RSAD2 (FIG. 4D) expression.

FIG. 5A-FIG. 5D are graphs showing the time dependent effects of sPIF and Mutant-10 sPIF in cultured HepaRG cells induced by TxpIC 500 ng/ml, on CXCL2 (FIG. 5A), IL28 (FIG. 5B), CCL5 (FIG. 5C), and RSAD2 (FIG. 5D).

FIG. 6A is a graph showing the sPIF effect on RSAD2 in Hepatitis C (HCV)-induced primary human hepatocytes (PHH). FIG. 6B-FIG. 6C are graphs showing sPIF Mutant 3 and Mutant 10 effects on RSAD2 (FIG. 6B) and CxCL10 (FIG. 6C) in TpxIC-induced PHH.

FIG. 7A is a graph showing the sPIF effect on IL28 in TxiPC and HCV-induced primary human hepatocytes (PHH). FIG. 7B-FIG. 7C are graphs showing sPIF Mutant 3 and Mutant 10 effects on IL28 (FIG. 7B) and CC15 (FIG. 7C) in TpxIC-induced PHH.

FIG. 8A is a graph showing sPIF Mutant 3 and Mutant 10 effects on IL28 in HCV-induced PHH. FIG. 8B is a graph showing sPIF Mutant 3 and Mutant 10 effects on CXCL10 in HCV-induced PHH. FIG. 8C is a graph showing sPIF effects on IP10 in TpxIC and HCV-induced PHH.

FIG. 9A-FIG. 9D are graphs showing sPIF effects on IP10 (FIG. 9A), CCL5 (FIG. 9B), RSAD2 (FIG. 9C), and IL1B (FIG. 9D) in HCV-induced THP1 cells.

FIG. 10A is a picture showing sPIF effect on mouse weight after cytomegalovirus (CMV) infection. The first two mice on the left of the picture were infected with 750 PFU of EGFP mCMV on the DOB, the mouse on the right is a not infected one (control). The first mouse on the left received PBS whereas the mouse in the middle received sPIF. A delayed body growth is detectable in the PBS-treated mouse, which is partially improved by sPIF treatment. FIG. 10B is a graph showing that treatment with sPIF increased survival rate in CMV-infected mice. Log-rank (Mantel-Cox) test for Kaplan-Meier survival curve analysis; p-value: 0.04. N=14-17 mice/experimental group.

FIG. 11A is a graph showing the sPIF effect on weight after CMV infection. Mice were weighted on postnatal day 10, the last day of sPIF or vehicle administration. Data are shown as mean±SD. N=7-9 mice/experimental group. **p-value<0.01. FIG. 11B is a graph showing quantification of cerebellar area (expressed as percentage of total brain area) after treatment with sPIF in mice with CMV infection. Data are shown as mean±SD. Measurements were taken from 5 brain sections/mouse, N=2 mice/experimental group. *p-value<0.05, NS: not significant.

FIG. 12A-FIG. 12B are graphs showing FITC-PIF (FIG. 12A) and FITC-PIFscr control (FIG. 12B) binding to white blood cells (WBC) in the hypercholesterolemic APO-E-model. FIG. 12C-FIG. 12D are graphs showing the effect of sPIF on CD4+ cells (FIG. 12C) and NKT+ cells (FIG. 12D) in the APO-E-model.

FIG. 13A-FIG. 13D are graphs showing sPIF effect on liver function enzymes AST (FIG. 13A), ALT (FIG. 13B), ALKP (FIG. 13C), and GGT (FIG. 13D) in streptozocin-induced hepatotoxicity.

FIG. 14A-FIG. 14C are graphs showing sPIF effect on kidney BUN (FIG. 14A), creatinine (FIG. 14B), and albumin (FIG. 14C) in streptozocin-induced hepatotoxicity.

### DETAILED DESCRIPTION OF EMBODIMENTS

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must also be noted that as used herein and in the appended claims, the singular forms “a”, “an”, and “the” include plural reference unless the context clearly dictates otherwise. Thus, for example, reference to a “peptide” is a reference to one or more peptides and equivalents thereof known to those skilled in the art, and so forth. In some embodiments, the peptide has a percent homology or sequence identity to any one of the sequences disclosed herein. The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may he performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

By “substantially identical” is meant nucleic acid molecule (or polypeptide) exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison. In some embodiments, the PIF peptide comprises about 70%, 75%, 80%, 85%, 87%, 90%, 91%, 92%, 93%, 94%, 95%, 96%, 97%, 98%, 99% or 100% sequence identity to any of the sequences disclosed herein. In some embodiments, the PIF peptide comprises about 70%, 75%, 80%, 85%, 87%, 90%, 91%, 92%, 93%, 94%, 95%, 96%, 97%, 98%, 99% or 100% sequence identity to any of the sequences disclosed in Table 1. In some embodiments, the peptidomimetics of PIF comprises about 70%, 75%, 80%, 85%, 87%, 90%, 91%, 92%, 93%, 94%, 95%, 96%, 97%, 98%, 99% or 100% sequence identity to any of the sequences disclosed in Table 1 and share at least about 50% biological activity of the wild-type sequence upon which it is based. In some embodiments, the peptidomimetics of PIF comprises about 70%, 75%, 80%, 85%, 87%, 90%, 91%, 92%, 93%, 94%, 95%, 96%, 97%, 98%, 99% or 100% sequence identity to any of the sequences disclosed in Table 1 and share at least 60% biological activity of the wild-type sequence upon which it is based. In some embodiments, the peptidomimetics of PIF comprises about 70%, 75%, 80%, 85%, 87%, 90%, 91%, 92%, 93%, 94%, 95%, 96%, 97%, 98%, 99% or 100% sequence identity to any of the sequences disclosed in Table 1 and share at least about 70% biological activity of the wild-type sequence upon which it is based.

In some embodiments, the peptidomimetics of PIF comprises about 70%, 75%, 80%, 85%, 87%, 90%, 91%, 92%, 93%, 94%, 95%, 96%, 97%, 98%, 99% or 100% sequence identity to any of the sequences disclosed in Table 1 and share at least about 80% biological activity of the wild-type sequence upon which it is based.
In some embodiments, the peptidomimetics of PIF comprises about 70%, 75%, 80%, 85%, 87%, 90%, 91%, 92%, 93%, 94%, 95%, 96%, 97%, 98%, 99% or 100% sequence identity to any of the sequences disclosed in Table 1 and share at least about 90% biological activity of the wild-type sequence upon which it is based. In some embodiments, the peptidomimetics of PIF comprises about 70%, 75%, 80%, 85%, 87%, 90%, 91%, 92%, 93%, 94%, 95%, 96%, 97%, 98%, 99% or 100% sequence identity to any of the sequences disclosed in Table 1 and share at least about 100% biological activity of the wild-type sequence upon which it is based.
In some embodiments, the peptidomimetics of PIF comprises about 70%, 75%, 80%, 85%, 87%, 90%, 91%, 92%, 93%, 94%, 95%, 96%, 97%, 98%, 99% or 100% sequence identity to the amino acid sequence RIKP and share at least about 70%, 80%, 90% or at least about 100% biological activity of the wild-type sequence upon which it is based. In some embodiments, the peptidomimetics of PIF comprises about 70%, 75%, 80%, 85%, 87%, 90%, 91%, 92%, 93%, 94%, 95%, 96%, 97%, 98%, 99% or 100% sequence identity to any of the sequences disclosed in Table 1 and share at least about 100% biological activity of the wild-type sequence upon which it is based. In some embodiments, the biological activity is measured as a function of expression of inflammatory cytokines or chemokines or combinations of both by the cells that interact with peptide. In some embodiments, the biological activity of a PIF peptide is measured by its anti-inflammatory effect on tissues or tissue or cells from a subject to which the peptide has been administered.

As used herein, the term “about” means plus or minus 20%, 10% or 5% of the numerical value of the number with which it is being disclosed. Therefore, about 50% means in the range of 40%-60%.

“Administering” when used in conjunction with a therapeutic means to administer a therapeutic directly into or onto a target organ, tissue or cell or to administer a therapeutic to a patient, whereby the therapeutic positively impacts the organ, tissue or cell to which it is targeted. Thus, as used herein, the term “administering”, when used in conjunction with preimplantation factor (PIF), can include, but is not limited to, providing PIF into or onto the target organ, tissue or cell; providing PIF systemically to a patient by, e.g., intravenous injection whereby the therapeutic reaches the target organ, tissue or cell; providing PIF in the form of the encoding sequence thereof to the target tissue (e.g., by so-called gene-therapy techniques). “Administering” may be accomplished by parenteral, oral or topical administration, or by such methods in combination with other known techniques.

The terms “animal,” “patient,” and “subject” as used herein include, but are not limited to, humans and non-human vertebrates such as wild, domestic and farm animals. In some embodiments, the terms “animal,” “patient,” and “subject” may refer to humans. In some embodiments, the terms “animal,” “patient,” and “subject” may refer to non-human mammals. In some embodiments, the terms “animal,” “patient,” and “subject” may refer to any or combination of: dogs, cats, pigs, cows, horses, goats, sheep or other domesticated non-human mammals. In some embodiments, the subject is a human patient that has been diagnosed or is suspected of having a non-alcoholic fatty liver disease (NAFLD). In some embodiments, the subject is a human patient that has been diagnosed or is suspected of having non-alcoholic steatohepatitis (NASH). In some embodiments, the subject is a human patient that has been diagnosed or is suspected of having NASH-induced fibrosis or fibrosis of the liver.

“Immunomodulating” refers to the ability of a compound of the present invention to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

The term “improves” is used to convey that the present invention changes either the appearance, form, characteristics and/or the physical attributes of the subject, organ, tissue or cell to which it is being provided, applied or administered. For example, the change in form may be demonstrated by any of the following alone or in combination: a decrease in one or more symptoms of a disease or disorder; reduction or elimination of the need for immune suppressive agents; and faster recovery from a disease or disorder.

The term “inhibiting” includes the administration of a compound of the present invention to prevent the onset of the symptoms, alleviate the symptoms, or eliminate the disease, condition or disorder.

As used herein, the terms “peptide,” “polypeptide” and “protein” are used interchangeably and refer to two or more amino acids covalently linked by an amide bond or non-amide equivalent. The peptides of the invention can be of any length. For example, the peptides can have from about two to about 100 or more residues, such as, 5 to 12, 12 to 15, 15 to 18, 18 to 25, 25 to 50, 50 to 75, 75 to 100, or more in length. Preferably, peptides are from about 2 to about 18 residues. The peptides of the invention include L- and D-isomers, and combinations of L- and D-isomers. The peptides can include modifications typically associated with post-translational processing of proteins, for example, cyclization (e.g., disulfide or amide bond), phosphorylation, glycosylation, carboxylation, ubiquitination, myristylation, or lipidation. In some embodiments, the peptides of the disclosure comprise only D-isomers. In some embodiments, the peptides comprise only L-isomers.

By “pharmaceutically acceptable,” it is meant the carrier, diluent or excipient must be compatible with the other ingredients of the formulation or composition and not deleterious to the recipient thereof.

As used herein, the term “therapeutic” means an agent utilized to treat, combat, ameliorate, prevent or improve an unwanted condition or disease of a patient. In part, embodiments of the present invention are directed to decreasing one or more symptoms of non-alcoholic fatty liver disease (NAFLD) or non-alcoholic steatohepatitis (NASH).

A “therapeutically effective amount” or “effective amount” of a composition (e.g., a PIF peptide) is a predetermined amount calculated to achieve the desired effect, i.e., to treat, combat, ameliorate, prevent or improve one or more symptoms of non-alcoholic fatty liver disease (NAFLD) or non-alcoholic steatohepatitis (NASH) and/or fibrosis. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. The compounds are effective over a wide dosage range and, for example, dosages per day will normally fall within the range of from about 0.001 μg to about 10 mg/kg, usually in the range of from about 0.01 μg to about 5 mg/kg. In some embodiments, the therapeutically effective dose of PIF or a PIF analog or peptide is about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, and about 1 mg/kg. However, it will be understood that the effective amount administered will be determined by the physician in the light of the relevant circumstances including the condition to be treated, the choice of compound to be administered, and the chosen route of administration, and therefore the above dosage ranges are not intended to limit the scope of the invention in any way. A therapeutically effective amount of compounds of embodiments of this invention is typically an amount such that when it is administered in a physiologically tolerable excipient composition, it is sufficient to achieve an effective systemic concentration or local concentration in the tissue.

The terms “treat,” “treated,” or “treating” as used herein refers to both therapeutic treatment and/or prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

Generally speaking, the term “tissue” refers to any aggregation of similarly specialized cells which are united in the performance of a particular function.

The disclosure relates to compositions and pharmaceutical compositions comprising polypeptides described herein, which, in some embodiments, act as agonists of PIF-mediated signal transduction via the receptor or receptors of PIF. These compositions or pharmaceutical compositions modulate signaling pathways that provide significant therapeutic benefit in the treatment or prevention of non-alcoholic fatty liver disease (NAFLD), including non-alcoholic steatohepatitis (NASH). The compositions of the present disclosure may exist in unsolvated forms as well as solvated forms, including hydrated forms of the polypeptides disclosed herein. The compositions of the present disclosure also are capable of forming both pharmaceutically acceptable salts, including but not limited to acid addition and/or base addition salts. Furthermore, compositions of the present disclosure may exist in various solid states including an amorphous form (non-crystalline form), and in the form of clathrates, prodrugs, polymorphs, bio-hydrolyzable esters, racemic mixtures, non-racemic mixtures, or as purified stereoisomers including, but not limited to, optically pure enantiomers and diastereomers. In general, all of these forms can be used as an alternative form to the free base or free acid forms of the compounds, as described above and are intended to be encompassed within the scope of the present disclosure.

A “polymorph” refers to solid crystalline forms of a compound. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Different physical properties of polymorphs can affect their processing. In some embodiments, the pharmaceutical composition comprises at least one polymorph of any of the compositions disclosed herein.

As noted above, the compositions or pharmaceutical compositions of the present disclosure can be administered, inter alia, as pharmaceutically acceptable salts, esters, amides or prodrugs. The term “salts” refers to inorganic and organic salts of compounds of the present disclosure. The salts can be prepared in situ during the final isolation and purification of a compound, or by separately reacting a purified compound in its free base or acid form with a suitable organic or inorganic base or acid and isolating the salt thus formed. Representative salts include the hydrobromide, hydrochloride, sulfate, bisulfate, nitrate, acetate, oxalate, palmitiate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. The salts may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, for example, S. M. Berge, et al., “Pharmaceutical Salts,” J Pharm Sci, 66: 1-19 (1977). The term “salt” refers to acidic salts formed with inorganic and/or organic acids, as well as basic salts formed with inorganic and/or organic bases. Examples of these acids and bases are well known to those of ordinary skill in the art. Such acid addition salts will normally be pharmaceutically acceptable although salts of non-pharmaceutically acceptable acids may be of utility in the preparation and purification of the compound in question. Salts include those formed from hydrochloric, hydrobromic, sulphuric, phosphoric, citric, tartaric, lactic, pyruvic, acetic, succinic, fumaric, maleic, methanesulphonic and benzenesulphonic acids.

In some embodiments, salts of the compositions comprising either a PIF or PIF analog or PIF mutant may be formed by reacting the free base, or a salt, enantiomer or racemate thereof, with one or more equivalents of the appropriate acid. In some embodiments, pharmaceutical acceptable salts of the present disclosure refer to analogs having at least one basic group or at least one basic radical. In some embodiments, pharmaceutical acceptable salts of the present disclosure comprise a free amino group, a free guanidino group, a pyrazinyl radical, or a pyridyl radical that forms acid addition salts. In some embodiments, the pharmaceutical acceptable salts of the present disclosure refer to analogs that are acid addition salts of the subject compounds with (for example) inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxybenzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid. When several basic groups are present mono- or poly-acid addition salts may be formed. The reaction may be carried out in a solvent or medium in which the salt is insoluble or in a solvent in which the salt is soluble, for example, water, dioxane, ethanol, tetrahydrofuran or diethyl ether, or a mixture of solvents, which may be removed in vacuo or by freeze drying. The reaction may also be a metathetical process or it may be carried out on an ion exchange resin. In some embodiments, the salts may be those that are physiologically tolerated by a patient. Salts according to the present disclosure may be found in their anhydrous form or as in hydrated crystalline form (i.e., complexed or crystallized with one or more molecules of water).

Examples of pharmaceutically acceptable esters of the compounds of the present disclosure include C₁-C₈ alkyl esters. Acceptable esters also include C₅-C₇ cycloalkyl esters, as well as arylalkyl esters such as benzyl. C₁-C₄ alkyl esters are commonly used. Esters of compounds of the present disclosure may be prepared according to methods that are well known in the art. Examples of pharmaceutically acceptable amides of the compounds of the present disclosure include amides derived from ammonia, primary C₁-C₈ alkyl amines, and secondary C₁-C₈ dialkyl amines. In the case of secondary amines, the amine may also be in the form of a 5 or 6 membered heterocycloalkyl group containing at least one nitrogen atom. Amides derived from ammonia, C₁-C₃ primary alkyl amines and C₁-C₂ dialkyl secondary amines are commonly used. Amides of the compounds of the present disclosure may be prepared according to methods well known to those skilled in the art.

As used herein, “conservative” amino acid substitutions may be defined as set out in Tables A, B, or C below. The PIF compounds of the disclosure include those wherein conservative substitutions (from either nucleic acid or amino acid sequences) have been introduced by modification of polynucleotides encoding polypeptides of the disclosure. Amino acids can be classified according to physical properties and contribution to secondary and tertiary protein structure. A conservative substitution is recognized in the art as a substitution of one amino acid for another amino acid that has similar properties. In some embodiments, the conservative substitution is recognized in the art as a substitution of one nucleic acid for another nucleic acid that has similar properties, or, when encoded, has equivalent or similar binding affinities.

The disclosure also relates to compositions and pharmaceutical compositions comprising one or a plurality of amino acid structural and functional analogs, for example, peptidomimetics having synthetic or non-natural amino acids (such as a norleucine) or amino acid analogues or non-natural side chains, so long as the mimetic shares one or more functions or activities of compounds of the disclosure. The compounds of the disclosure therefore include “mimetic” and “peptidomimetic” forms. As used herein, a “non-natural side chain” is a modified or synthetic chain of atoms joined by covalent bond to the α-carbon atom, β-carbon atom, or γ-carbon atom which does not make up the backbone of the polypeptide chain of amino acids. The peptide analogs may comprise one or a combination of non-natural amino-acids chosen from: norvaline, tert-butylglycine, phenylglycine, He, 7-azatryptophan, 4-fluorophenylalanine, N-methyl-methionine, N-methyl-valine, N-methyl-alanine, sarcosine, N-methyl-tert-butylglycine, N-methyl-leucine, N-methyl-phenylglycine, N-methyl-isoleucine, N-methyl-tryptophan, N-methyl-7-azatryptophan, N-methyl-phenylalanine, N-methyl-4-fluorophenylalanine, N-methyl-threonine, N-methyl-tyrosine, N-methyl-valine, N-methyl-lysine, homocysteine, and Tyr; Xaa2 is absent, or an amino acid selected from the group consisting of Ala, D-Ala, N-methyl-alanine, Glu, N-methyl-glutamate, D-Glu, Gly, sarcosine, norleucine, Lys, D-Lys, Asn, D-Asn, D-Glu, Arg, D-Arg, Phe, D-Phe, N-methyl-phenylalanine, Gin, D-Gln, Asp, D-Asp, Ser, D-Ser, N-methyl-serine, Thr, D-Thr, N-methyl-threonine, D-Pro D-Leu, N-methyl-leucine, D-Ile, N-methyl-isoleucine, D-Val, N-methyl-valine, tert-butylglycine, D-tert-butylglycine, N-methyl-tert-butylglycine, Trp, D-Trp, N-methyl-tryptophan, D-Tyr, N-methyl-tyrosine, 1-aminocyclopropanecarboxylic acid, 1-aminocyclobutanecarboxylic acid, 1-aminocyclopentanecarboxylic acid, 1-aminocyclohexanecarboxylic acid, 4-aminotetrahydro-2H-pyran-4-carboxylic acid, aminoisobutyric acid, (5)-2-amino-3-(IH-tetrazol-5-yl)propanoic acid, Glu, Gly, N-methyl-glutamate, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, octylglycine, tranexamic acid, aminovaleric acid, and 2-(2-aminoethoxy)acetic acid. The natural side chain, or R group, of an alanine is a methyl group. In some embodiments, the non-natural side chain of the composition is a methyl group in which one or more of the hydrogen atoms is replaced by a deuterium atom. Non-natural side chains are disclosed in the art in the following publications: WO/2013/172954, WO2013123267, WO/2014/071241, WO/2014/138429, WO/2013/050615, WO/2013/050616, WO/2012/166559, US Application No. 20150094457, Ma, Z., and Hartman, M. C. (2012). In Vitro Selection of Unnatural Cyclic Peptide Libraries via mRNA Display. In J. A. Douthwaite & R. H. Jackson (Eds.), Ribosome Display and Related Technologies: Methods and Protocols (pp. 367-390). Springer New York, all of which are incorporated by reference in their entireties.

The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinant or chemically modified peptides, as well as non-peptide agents such as small molecule drug mimetics, as further described below. The term “analog” refers to any polypeptide comprising at least one α-amino acid and at least one non-native amino acid residue, wherein the polypeptide is structurally similar to a naturally occurring full-length PIF protein and shares the biochemical or biological activity of the naturally occurring full-length protein upon which the analog is based. In some embodiments, the compositions, pharmaceutical compositions and kits comprise a peptide or peptidomimetic sharing share no less than about 70%, about 75%, about 79%, about 80%, about 85%, about 86%, about 87%, about 90%, about 93%, about 94% about 95%, about 96%, about 97%, about 98%, about 99% homology with any one or combination of PIF sequences; and wherein one or a plurality of amino acid residues is a non-natural amino acid residue or an amino acid residue with a non-natural sidechain. In some embodiments, peptide or peptide mimetics are provided, wherein a loop is formed between two cysteine residues. In some embodiments, the peptidomimetic may have many similarities to natural peptides, such as: amino acid side chains that are not found among the known 20 proteinogenic amino acids, non-peptide-based linkers used to effect cyclization between the ends or internal portions of the molecule, substitutions of the amide bond hydrogen moiety by methyl groups (N-methylation) or other alkyl groups, replacement of a peptide bond with a chemical group or bond that is resistant to chemical or enzymatic treatments, N- and C-terminal modifications, and conjugation with a non-peptidic extension (such as polyethylene glycol, lipids, carbohydrates, nucleosides, nucleotides, nucleoside bases, various small molecules, or phosphate or sulfate groups). As used herein, the term “cyclic peptide mimetic” or “cyclic polypeptide mimetic” refers to a peptide mimetic that has as part of its structure one or more cyclic features such as a loop, bridging moiety, and/or an internal linkage. As used herein, the term “bridging moiety” refers to a chemical moiety that chemically links one or a combination of atoms on an amino acid to any other atoms outside of the amino acid residue. For instance, in the case of amino acid tertiary structure, a bridging moiety may be a chemical moiety that chemically links one amino acid side chain with another sequential or non-sequential amino acid side chain.

Ultimately, a novel embryo-derived peptide, PIF, creates a tolerogenic state at low doses following short-term treatment leading to long-term protection from tissue rejection after transplantation. This effect is exerted without apparent toxicity and is exerted, in some embodiment, as a monotherapy without other active agents that may modulate the immune system. In some embodiments, the methods of treatment are performed without administration of steroids. In some embodiments, the methods of treatment are performed without administration of any other therapeutic compound.

For therapeutic treatment of the specified indications, a PIF peptide may be administered as such, or can be compounded and formulated into pharmaceutical compositions in unit dosage form for parenteral, transdermal, rectal, nasal, local intravenous administration, or, preferably, oral administration. Such pharmaceutical compositions are prepared in a manner well known in the art and comprise at least one active PIF peptide associated with a pharmaceutically carrier. The term “active compound”, as used throughout this specification, refers to at least one composition comprising one or a plurality of selected from compounds of the formulas or pharmaceutically acceptable salts thereof. PIF peptides may be any one or combination of those peptides from Table 1.

In such a composition, the active compound is known as the “active ingredient.” In making the compositions, the active ingredient will usually be mixed with a carrier, or diluted by a carrier, or enclosed within a carrier that may be in the form of a capsule, sachet, paper or other container. When the carrier serves as a diluent, it may be a solid, semisolid, or liquid material that acts as a vehicle, excipient of medium for the active ingredient. Thus, the composition can be in the form of tablets, pills, powders, lozenges, sachets, cachets, elixirs, emulsion, solutions, syrups, suspensions, soft and hard gelatin capsules, sterile injectable solutions, and sterile packaged powders.

The terms “pharmaceutical preparation” and “pharmaceutical composition” include preparations suitable for administration to mammals, e.g., humans. When the compounds of the present disclosure are administered as pharmaceuticals to mammals, e.g., humans, they can be given per se or as a pharmaceutical composition containing, for example, from about 0.1 to about 99.5% of active ingredient in combination with a pharmaceutically acceptable carrier.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. In some embodiments, the pharmaceutical compositions comprising a PIF peptide, mimetic or pharmaceutically acceptable salt thereof and at least one pharmaceutically acceptable carrier.

The phrase “pharmaceutically acceptable carrier” is art recognized and includes a pharmaceutically acceptable material, composition or vehicle, suitable for administering compounds of the present disclosure to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in “Remington's Pharmaceutical Sciences” by E. W. Martin, which is incorporated herein by reference in its entirety. In some embodiments, the pharmaceutically acceptable carrier is sterile and pyrogen-free water. In some embodiments, the pharmaceutically acceptable carrier is Ringer's Lactate, sometimes known as lactated Ringer's solution.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, .alpha.-tocopherol, and the like; and metal chelating agents, such as citric acid, ethylenediamine tetraacetic acid (EDTA), sorbitol, tartaric acid, phosphoric acid, and the like.

Formulations of the present disclosure include those suitable for oral, nasal, topical, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Some examples of suitable carriers, excipients, and diluents include lactose, dextrose, sucrose, sorbitol, mannitol, starches, gum acacia, calcium phosphate alginates, calcium salicate, microcrystalline cellulose, polyvinylpyrrolidone, cellulose, tragacanth, gelatin, syrup, methyl cellulose, methyl- and propylhydroxybenzoates, tale, magnesium stearate, water, and mineral oil. The formulations can additionally include lubricating agents, wetting agents, emulsifying and suspending agents, preserving agents, sweetening agents or flavoring agents. The compositions may be formulated so as to provide quick, sustained, or delayed release of the active ingredient after administration to the patient by employing procedures well known in the art.

For oral administration, a compound can be admixed with carriers and diluents, molded into tablets, or enclosed in gelatin capsules. The mixtures can alternatively be dissolved in liquids such as 10% aqueous glucose solution, isotonic saline, sterile water, or the like, and administered intravenously or by injection.

The local delivery of therapeutically effective amounts of active compound for the treatment of non-alcoholic fatty liver disease (NAFLD) can be by a variety of techniques that administer the compound at or near the targeted site. Examples of local delivery techniques are not intended to be limiting but to be illustrative of the techniques available. Examples include local delivery catheters, site specific carriers, implants, direct injection, or direct applications, such as topical application.

Local delivery by an implant describes the surgical placement of a matrix that contains the pharmaceutical agent into the affected site. The implanted matrix releases the pharmaceutical agent by diffusion, chemical reaction, or solvent activators.

For example, in some aspects, the disclosure is directed to a pharmaceutical composition comprising a PIF peptide, and a pharmaceutically acceptable carrier or diluent, or an effective amount of pharmaceutical composition comprising a PIF peptide.

TABLE 1
PIF Peptides

| (SEQ ID NO) | Peptide | Amino Acid Sequence |
|---|---|---|
| SEQ ID NO: 1 | nPIF-1₁₅ | MVRIKPGSANKPSDD |
| isolated native, matches region of | | |
| Circumsporozoite protein (Malaria) | | |
| SEQ ID NO: 2 | nPIF-1₍₁₅₋ₐₗₜₑᵣ₎ | MVRIKYGSYNNKPSD |
| isolated native, matches region of | | |
| Circumsporozoite protein (Malaria) | | |
| SEQ ID NO: 3 | nPIF-1₍₁₃₎ | MVRIKPGSANKPS |
| isolated native, matches region of | | |
| Circumsporozoite protein (Malaria) | | |
| SEQ ID NO: 4 | nPIF-1₍₉₎ | MVRIKPGSA |
| isolated native, matches region of | | |
| Circumsporozoite protein (Malaria) | | |
| SEQ ID NO: 5 | scrPIF-1₁₅ | GRVDPSNKSMPKDIA |
| synthetic, scrambled amino acid sequence | | |
| from region of Circumsporozoite protein | | |
| Malaria | | |
| SEQ ID NO: 6 | nPIF-2₍₁₀₎ | SQAVQEHAST |
| isolated native, matches region of human | | |
| retinoid and thyroid hormone receptor-SMRT | | |
| SEQ ID NO: 7 | nPIF-2₍₁₃₎ | SQAVQEHASTNMG |
| isolated native, matches region of human | | |
| retinoid and thyroid hormone receptor (SMRT) | | |
| SEQ ID NO: 8 | scrPIF-2₍₁₃₎ | EVAQHSQASTMNG |
| synthetic, scrambled amino acid sequence | | |
| from region of human retinoid and thyroid | | |
| hormone receptor SMRT | | |
| SEQ ID NO: 9 | scrPIF-2₍₁₄₎ | GQASSAQMNSTGVH |
| SEQ ID NO: 10 | nPIF-3₍₁₈₎ | SGIVIYQYMDDRYVGSDL |
| isolated native, matches region of Rev Trans | | |
| SEQ ID NO: 11 | Neg control for | GMRELQRSANK |
| synthetic, scrambled amino acid sequence | negPIF-1₍₁₅₎ | |
| from region of Circumsporozoite protein | | |
| Malaria | | |
| SEQ ID NO: 12 | nPIF-4₍₉₎ | VIHAQYMD |
| isolated native, matches region of Rev Trans | | |
| antibody of native isolated nPIF-115 | AbPIF-1₍₁₅₎ | |
| (SEQ ID NO: 13) | sPIF-1₍₁₅₎ | MVRIKPGSANKPSDD |
| synthetic, amino acid sequence from region of | | |
| Circumsporozoite protein Malaria | | |
| (SEQ ID NO: 14) | sPIF-2₍₁₃₎ | SQAVQEHASTNMG |
| synthetic, amino acid sequence from of human | | |
| retinoid and thyroid hormone receptor SMRT | | |
| (SEQ ID NO: 15) | sPIF-3₍₁₈₎ | SGIVIYQYMDDRYVGSDL |
| synthetic, amino acid sequence from region of | | |
| Circumsporozoite protein Malaria | | |
| (SEQ ID NO: 16) | sPIF-1₍₉₎ | MVRIKPGSA |
| synthetic, amino acid sequence from region of | | |
| Circumsporozoite protein Malaria | | |
| antibody of native isolated nPIF-2₍₁₃₎ | AbPIF-2₍₁₃₎ | |
| antibody of native isolated nPIF-3₍₁₈₎ | AbPIF-3₍₁₈₎ | |
| (SEQ ID NO: 17) | sPIF-4₍₉₎ | VIHAQYMD |
| Synthetic | | |
| SEQ ID NO: 18 | sPIF-1₍₅₎ | MVRIK |
| Synthetic | | |
| SEQ ID NO: 19 | sPIF-1₍₄₎ | PGSA |
| Synthetic | | |
| SEQ ID NO: 20 | PIF (−3) | MVXIKPGSANKPSDD |
| SEQ ID NO: 21 | PIF (−1) | XVRIKPGSANKPSDD |
| SEQ ID NO: 22 | PIF (−1, −3) | XVXIKPGSANKPSDD |
| SEQ ID NO: 23 | PIF (−6) | MVRIKXGSANKPSDD |
| SEQ ID NO: 24 | PIF (−4) | MVRXKPGSANKPSDD |
| SEQ ID NO: 25 | PIF (−2) | MXRIKPGSANKPSDD |
| SEQ ID NO: 26 | mut1 | MVRIKEGSANKPSDD |
| SEQ ID NO: 27 | mut3 | MVRGKPGSANKPSDD |
| SEQ ID NO: 28 | mut4 | MERIKPGSANKPSDD |
| SEQ ID NO: 29 | mut5 | AVRIKPGSANKPSDD |

n = native, s = synthetic, scr = scrambled, same AA, ( ) = number of AA, Ab = antibody,
X = any amino acid, except arginine

Specific modes of administration will depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered will depend on the characteristics of the subject being treated, e.g., the particular mammal or human treated, age, weight, health, types of concurrent treatment, if any, and frequency of treatments, and can be easily determined by one of skill in the art (e.g., by the clinician).

Pharmaceutical formulations containing the compounds of the present disclosure and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels, jellies, and foams; and parenteral dosage forms which include, but are not limited to, solutions, suspensions, emulsions, and dry powder; comprising an effective amount of a polymer or copolymer of the present disclosure. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compositions or pharmaceutical compositions of the present disclosure can be formulated for parenteral administration by injection, e.g., by bolus injection or continuous infusion. The compounds can be administered by continuous infusion subcutaneously over a predetermined period of time. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compounds can be formulated readily by combining these compounds with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, alter adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragecanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include, but are not limited to, push-fit capsules made of gelatin, as well as soft, scaled capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds of the present disclosure can also be formulated in rectal compositions such as suppositories or retention enemas, e.g., containing conventional suppository bases such as cocoa butter or other glycerides.

In addition to the formulations described previously, the compounds of the present disclosure can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of the present disclosure, for example, can be applied to a plaster, or can be applied by transdermal, therapeutic systems that are consequently supplied to the organism.

Pharmaceutical compositions comprising any one or plurality of compounds disclosed herein also can comprise suitable solid or gel phase carriers or excipients. Examples of such carriers or excipients include but are not limited to calcium carbonate, calcium phosphate, various sugars, starches, cellulose derivates, gelatin, and polymers such as, e.g., polyethylene glycols.

For parenteral administration, a composition or pharmaceutical composition can be, for example, formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques. For example, a parenteral composition suitable for administration by injection is prepared by dissolving 1.5% by weight of analog in 0.9% sodium chloride solution.

The present disclosure relates to routes of administration include intramuscular, sublingual, intravenous, intraperitoneal, intrathecal, intravaginal, intraurethral, intradermal, intrabuccal, via inhalation, via nebulizer and via subcutaneous injection. Alternatively, the pharmaceutical composition may be introduced by various means into cells that are removed from the individual. Such means include, for example, microprojectile bombardment and liposome or other nanoparticle device.

Solid dosage forms for oral administration include capsules, tablets, pills, powders and granules. In solid dosage forms, the composition or pharmaceutical compositions are generally admixed with at least one inert pharmaceutically acceptable carrier such as sucrose, lactose, starch, or other generally regarded as safe (GRAS) additives. Such dosage forms can also comprise, as is normal practice, an additional substance other than an inert diluent, e.g., lubricating agent such as magnesium state. With capsules, tablets, and pills, the dosage forms may also comprise a buffering agent. Tablets and pills can additionally be prepared with enteric coatings, or in a controlled release form, using techniques know in the art.

Liquid dosage forms for oral administration include pharmaceutically acceptable emulsions, solutions, suspensions and syrups, with the elixirs containing an inert diluent commonly used in the art, such as water. These compositions can also include one or more adjuvants, such as wetting agent, an emulsifying agent, a suspending agent, a sweetening agent, a flavoring agent or a perfuming agent.

In some embodiments, the disclosure relates to methods of modulating levels of liver enzymes in a subject, such as a human. In some embodiments, the disclosure relates to a method of reducing ALT levels in a human subject comprising administering a therapeutically effective amount of a PIF peptide to the subject in need thereof. In some embodiments, the disclosure relates to a method of decreasing the levels of any of the liver enzymes disclosed herein comprising administering a therapeutically effective amount of a PIF peptide to the subject in need thereof.

In some embodiments, the disclosure relates to a method of treating non-alcoholic fatty liver disease (NAFLD) comprising administering to a subject in need thereof a pharmaceutical composition comprising: (i) a therapeutically effective amount of a PIF peptide, compositions thereof, mimetics thereof, pharmaceutically acceptable salts thereof, or combinations thereof of any of the foregoing; and (ii) one or a plurality of pharmaceutically acceptable carriers.

In some embodiments, the disclosure relates to a method of preventing non-alcoholic fatty liver disease (NAFLD) comprising administering to a subject in need thereof a pharmaceutical composition comprising: (i) a therapeutically effective amount of a PIF peptide, compositions thereof, mimetics thereof, pharmaceutically acceptable salts thereof, or combinations thereof of any of the foregoing; and (ii) one or a plurality of pharmaceutically acceptable carriers.

In some embodiments, the disclosure relates to a method of treating non-alcoholic steatohepatitis (NASH) comprising administering to a subject in need thereof a pharmaceutical composition comprising: (i) a therapeutically effective amount of a PIF peptide, compositions thereof, mimetics thereof, pharmaceutically acceptable salts thereof, or combinations thereof of any of the foregoing; and (ii) one or a plurality of pharmaceutically acceptable carriers.

In some embodiments, the disclosure relates to a method of preventing non-alcoholic steatohepatitis (NASH) comprising administering to a subject in need thereof a pharmaceutical composition comprising: (i) a therapeutically effective amount of a PIF peptide, compositions thereof, mimetics thereof, pharmaceutically acceptable salts thereof, or combinations thereof of any of the foregoing; and (ii) one or a plurality of pharmaceutically acceptable carriers.

In some embodiments, an anti-inflammatory compound may be administered at least one hour prior to, simultaneously with, or subsequent to administration of compositions disclosed herein. In some embodiments, the anti-inflammatory compound is and anti-inflammatory compound listed in Table 5. In some embodiments, the anti-inflammatory compound is free of a steroid. In some embodiments, the therapy is a monotherapy. In some embodiments, the therapy also simultaneously treats a subject susceptible to or diagnosed with or having NAFLD, NASH, autoimmune hepatitis and/or Type I adult or juvenile diabetes.

One of skill in the art will recognize that the appropriate dosage of the compositions and pharmaceutical compositions may vary depending on the individual being treated and the purpose. For example, the age, body weight, and medical history of the individual patient may affect the therapeutic efficacy of the therapy. Further, a lower dosage of the composition may be needed to produce a transient cessation of symptoms, while a larger dose may be needed to produce a complete cessation of symptoms associated with the disease, disorder, or indication. A competent physician can consider these factors and adjust the dosing regimen to ensure the dose is achieving the desired therapeutic outcome without undue experimentation. It is also noted that the clinician and/or treating physician will know how and when to interrupt, adjust, and/or terminate therapy in conjunction with individual patient response. Dosages may also depend on the strength of the particular composition, pharmaceutical composition, salt or analog chosen for the pharmaceutical composition.

The dose of the composition or pharmaceutical compositions may vary. The dose of the composition may be once per day. In some embodiments, multiple doses may be administered to the subject per day. In some embodiments, the total dosage is administered in at least two application periods. In some embodiments, the period can be an hour, a day, a month, a year, a week, or a two-week period. In an additional embodiment of the invention, the total dosage is administered in two or more separate application periods, or separate doses over the course of an hour, a day, a month, a year, a week, or a two-week period. In some embodiments, pharmaceutical compositions of the present disclosure can be administered once, twice, or three times in a 30 day period. In some embodiments, pharmaceutical compositions of the present disclosure can be administered once, twice, or three times in a 24 hour period.

Dosage may be measured in terms of mass amount of polypeptide, salt, or analog per liter of liquid formulation prepared. One skilled in the art can increase or decrease the concentration of the polypeptide, salt, or analog in the dose depending upon the strength of biological activity desired to treat or prevent any above-mentioned disorders associated with the treatment of subjects in need thereof. For instance, some embodiments of the invention can include up to 0.00001 grams of polypeptide, salt, or analog per 5 mL of liquid formulation and up to about 10 grams of polypeptide, salt, or analog per 5 mL of liquid formulation.

In some embodiments the pharmaceutical compositions of the claimed invention comprise at least one or a plurality of active agents other than the PIF peptide, polypeptide, salt, or analog of pharmaceutically acceptable salt thereof. In some embodiments the active agent is covalently linked to the PIF peptide or PIF polypeptide, salt, or analog disclosed herein optionally by a protease cleavable linker (including by not limited to Pro-Pro or Cituline-Valine di-α-amino acid linkers). In some embodiments, the one or plurality of active agents includes one or a combination of compounds chosen from: an anti-inflammatory compound, alpha-adrenergic agonist, antiarrhythmic compound, analgesic compound, and an anesthetic compound.

TABLE 5
Examples of anti-inflammatory compounds include:
aspirin
celecoxib
diclofenac
diflunisal
etodolac
ibuprofen
indomethacin
ketoprofen
ketorolac nabumetone
naproxen
oxaprozin
piroxicam
salsalate
sulindac
tolmetin
Examples of alpha-adrenergic agonists include:
Methoxamine
Methylnorepinephrine
Midodrine
Oxymetazoline
Metaraminol
Phenylephrine
Clonidine (mixed alpha2-adrenergic and imidazoline-I1 receptor agonist)
Guanfacine, (preference for alpha2A-subtype of adrenoceptor)
Guanabenz (most selective agonist for alpha2-adrenergic as opposed
to imidazoline-I1)
Guanoxabenz (metabolite of guanabenz)
Guanethidine (peripheral alpha2-receptor agonist)
Xylazine,
Tizanidine
Medetomidine
Methyldopa
Fadolmidine
Dexmedetomidine
Examples of antiarrhythmic compounds include:
Amiodarone (Cordarone, Pacerone)
Bepridil Hydrochloride (Vascor)
Disopyramide (Norpace)
Dofetilide (Tikosyn)
Dronedarone (Multaq)
Flecainide (Tambocor)
Ibutilide (Corvert)
Lidocaine (Xylocaine)
Procainamide (Procan, Procanbid)
Propafenone (Rythmol)
Propranolol (Inderal)
Quinidine (many trade names)
Sotalol (Betapace)
Tocainide (Tonocarid)
Examples of analgesic compound include:
codeine
hydrocodone (Zohydro ER),
oxycodone (OxyContin, Roxicodone),
methadone
hydromorphone (Dilaudid, Exalgo),
morphine (Avinza, Kadian, MSIR, MS Contin), and
fentanyl (Actiq, Duragesic)
Examples of anesthetic compounds include:
Desflurane
Isoflurane
Nitrous oxide
Sevoflurane
Xenon

The compounds of the present disclosure can also be administered in combination with other active ingredients, such as, for example, adjuvants, or other compatible drugs or compounds where such combination is seen to be desirable or advantageous in achieving the desired effects of the methods described herein.

In the foregoing embodiments, the PIF peptide may be administered at a dose of about 0.0001 mg/kg/day, about 0.001 mg/kg/day, about 0.01 mg/kg/day, about 0.1 mg/kg/day, about 0.5 mg/kg/day, about 0.75 mg/kg/day, about 1 mg/kg/day, about 2 mg/kg/day, about 3 mg/kg/day, about 4 mg/kg/day, about 6 mg/kg/day, about 8 mg/kg/day, about 10 mg/kg/day, about 15 mg/kg/day, about 20 mg/kg/day, or any range between any of these values, including endpoints. Such doses may be administered as a single dose or as divided doses in a single day. In some embodiments, if administered once a day, the daily regimen may be repeated over two days, three day, four days, five days, six days, 7 days or more. In some embodiments, the daily regimen or regimens are repeated once a month or once every other month. In some embodiments, the dosage regimen may begin at one dose such as 1 mg/kg for any period of time in days or months and then slowly become reduced over time or escalate depending upon the health of the subject.

In the foregoing embodiments, the composition or pharmaceutical composition may be administered once, for a limited period of time, or as a maintenance therapy (over an extended period of time until the condition is ameliorated, cured or for the life of the subject). A limited period of time may be for 1 week, 2 weeks, 3 weeks, 4 weeks and up to one year, including any period of time between such values, including endpoints. In some embodiments, the composition or pharmaceutical composition may be administered for about 1 day, for about 3 days, for about 1 week, for about 7 days, for about 10 days, for about 2 weeks, for about 18 days, for about 3 weeks, or for any range between any of these values, including endpoints

In the foregoing embodiments, the composition or pharmaceutical composition may be administered once daily, twice daily, three times daily, four times daily or more.

In some embodiments, the composition or pharmaceutical composition is administered or provided as a pharmaceutical composition comprising a PIF peptide, as defined above, and a pharmaceutically acceptable carrier or diluent, or an effective amount of a pharmaceutical composition comprising a compound as defined above. The methods disclosed herein can be used with any of the compounds, compositions, preparations, and kits disclosed herein.

In some embodiments, the pharmaceutical composition consists of a single active agent that is PIF, PIF analog, PIF mimetic or salt thereof.

In some aspects, the disclosure relates to a pharmaceutical composition comprising a PIF peptide, PIF analog, PIF mimetic or salt thereof, plus a therapeutically effective amount of another active agent. In some embodiments, the other active agent is an agent from Table 5. In some embodiments, the other active agent is a steroid.

All referenced journal articles, patents, and other publications are incorporated by reference herein in their entireties.

### EXAMPLES

### Example 1

Human Studies in Patients with AIH

The link between AIH and NAFLD is strong. Many patients have already fibrosis by the time of diagnosis. The patients are heavy steroid and immune suppressive drug users and may have also anti-nuclear antibodies. akahashi A., Abe K., Ohira H. (2014) Nonalcoholic Steatohepatitis-Autoimmune Hepatitis Overlap. In: Ohira H. (eds) Autoimmune Liver Diseases. Springer, Tokyo DOI https://doi.org/10.1007/978-4-431-54789-1_10. Therefore the study of sPIF in chronic patients that have been exposed for several years to liver toxins (corticosteroids, immune suppressive agents) can reveal important information on a continuum that some of these patients will progress to NAFLD. The goal of sPIF is intervening in a prodromal-NAFLD which may prevent progress to the disease. The Phase I trial aimed to address such a possibility. Human studies were conducted using sPIF in randomized, double-blinded, placebo-controlled single and multiple ascending dose studies in females with chronic autoimmune hepatitis (AIH) that in 30% had already liver fibrosis and type 2 diabetes-NAFLD precursor. sPIF effect was tested on patients with AIH-recognizing that by the time of diagnosis in 30% of cases the patients already have fibrosis which is also hallmark of NAFLD/NASH. The definition of these conditions due to the variability of the symptoms are diverse specifically as it relates to ALT and AST levels in the individual patients. Although high level of enzymes may be present in certain proportion of patients with NASH in 80% of cases the ALT levels are within normal limits. The concern is that as levels normalizes it is not due to improved patient condition but is a progress to fibrosis. The data with sPIF generated showing in certain cases a rapid decline in elevated ALT and some cases also AST as well as ALP and GGT indicates that the rapid decrease in inflammation perforce does not due to progression of fibrosis. It also suggests that sPIF may address the global liver not only the inflamed hepatocytes since when liver undergoes fibrosis it affects all liver compartments. This is also supported by 1. sPIF targets in the liver 2. Changes in genes expression following exposure to Hep C and importantly non-specific viral inflammation that sPIF regulated. 3 The data in CMV confirms.

### Single Ascending Dose (SAD)

Patients 18 to 75 years old of non-child bearing potential with documented (AIH) and compensated chronic liver disease under standard of care, were enrolled in a single ascending dose study, in 6 patients already having liver fibrosis. The purpose of the study was to establish the safety, tolerability, and pharmacokinetics of sPIF. The sPIF and the placebo groups were similar in terms of demographic and baseline characteristics. The diagnosis of AIH was documented at the screening evaluation by either a pre-treatment score ≥15 or a post-treatment score of ≥17 by the International Criteria for the Diagnosis of Autoimmune Hepatitis. Treatment with oral, immunosuppressive drug(s) was stabilized for at least 6 weeks prior to screening for this study. Permitted concomitant immune suppression medications included azathioprine ≤100 mg per day, budesonide 9≤mg per day, mycophenolate mofetil ≤3000 mg per day, prednisone ≤30 mg per day, ursodeoxycholic acid ≤1500 mg per day, and tacrolimus ≤6 mg per day.

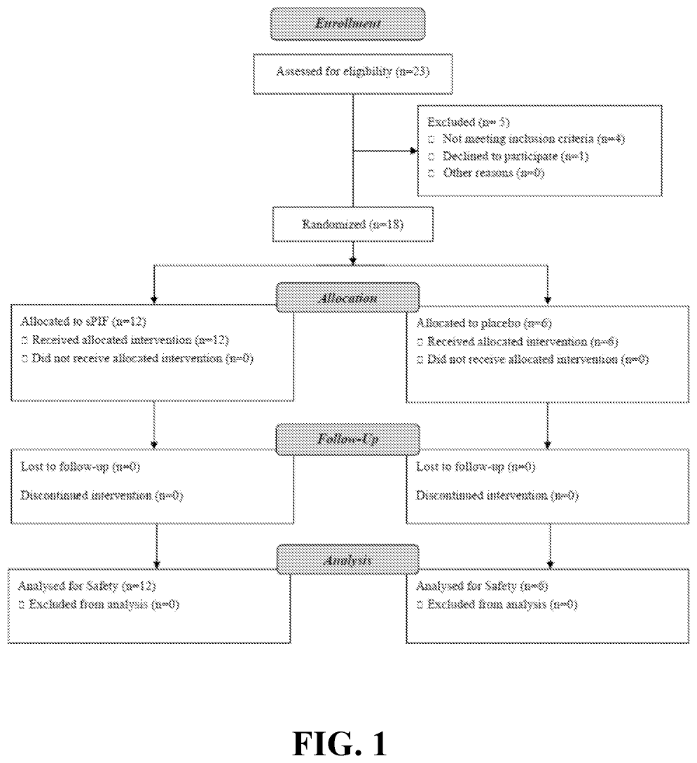

A total of two-hundred and fifty-two (252) patients were pre-screened. Twenty-three (23) were screened, eighteen (18) patients were enrolled, twelve (12) were randomly assigned to the active drug sPIF and (6) six to the placebo treatment arm. (FIG. 1) Patients were enrolled in three cohorts (0.1, 0.5 or 1.0 mg/kg) at a single US site. sPIF was reconstituted from powder to a 0.5 mL total volume per syringe with lactated Ringer's injection, USP (0.1, 0.5, 1 mg/kg) dose was adjusted to be appropriate for the patient's weight. Lactated Ringer's Injection, USP 0.5 mL was used as the placebo and was administered subcutaneously to subjects randomized to receive placebo under the same conditions as the active drug recipients. (Table 2)

TABLE 2
Demographics and baseline characteristics

| | placebo | sPIF | sPIF | sPIF | sPIF |
|---|---|---|---|---|---|
| | All | All | 0.1 mg/kg | 0.5 mg/kg | 1.0 mg/kg |
| Female, n (%) | 6 (100%) | 12(100%) | 4 (100%) | 4(100%) | 4(100%) |
| Age (y), mean +/− SD (mean) | 60 ± 11 | 60 ± 9 | 63 ± 10 | 59 ± 8 | 59 ± 9 |
| Ethnicity | | | | | |
| Caucasian, n | 3 (50%) | 11(91%) | 4 (100%) | 3 (75%) | 4(100%) |
| Hispanic, n | 1 (17%) | 1 (9%) | 0 (0%) | 1 (25%) | 0 (0%) |
| Black, n | 2 (33%) | 0 (0%) | 0 (0%) | 0 (0%) | 0 (0%) |
| Liver function tests | | | | | |
| Albumin (g/dL), mean +/− SD | 4.3 ± 0.3 | 4.3 ± 0.2 | 4.3 ± 0.2 | 4.3 ± 0.1 | 4.2 ± 0.3 |
| □ globulin (U/L), mean +/− SD | 3.8 ± 1.5 | 3.5 ± 0.3 | 3.4 ± 0.1 | 3.6 ± 0.3 | 3.5 ± 0.4 |
| Serologies | | | | | |
| Anti-HAV (positive), n | | | | | |
| Anti-HBsAg (positive), n | 0 | 0 | 0 | 0 | 0 |
| Anti-HCV (positive), n | 0 | 0 | 0 | 0 | 0 |
| AIH score, mean +/− SD (mean) | 18.2 ± 1.5 | 18.8 ± 1.2 | 18.8 ± 1.5 | 18.5 ± 1.0 | 19.0 ± 1.4 |

AIH, autoimmune hepatitis;
TB, total bilirubin;;
□ globulin, gamma globulin

### Safety and Tolerability

Safety and tolerability was evaluated by assessment of clinical laboratory tests, periodic physical examination, including vital signs measurements, 12-lead ECG at baseline (pre-dose, Day 1) and at various time points during the treatment phases of the protocol, and by the documentation of adverse events.

There was no grade 2, 3 or 4 adverse events in any of the 12 body systems reported by symptoms or physical exam in any patient to date during the active dosing portion of the study or in the 7 day follow-up period. One patient reported a headache that spontaneously resolved. One episode of “liver fullness” sensation transiently occurred in one patient. No other clinical side effects by history or physical exam were present in any dosing group. There was no grade 2, 3 or 4 adverse events of laboratory values in any patient to date during the single dose portion of the study or in the seven-day follow-up period. Minor, grade 1 or non-graded (World Health Organization criteria) changes in the blood studies occurred in the 7 patients from baseline values. There were no clinically significant laboratory results that required study drug to be modified, interrupted, or discontinued.

### Pharmacokinetics

Pharmacokinetic analysis was performed by determining sPIF levels in the plasma collected at baseline pre-dose, 30 (±5), 60 (±10) and 120 (±15) and 240 (±20) minutes after SQ injection using validated liquid chromatography and mass spectroscopy (LC-MS/MS) assay (lower limit of quantification 1 ng/mL). Assay was performed at (Covance Laboratories, WI) using a GLP validated assay. Since sPIF is administered subcutaneously, absorption into the systemic vascular system will occur. Post distribution serum concentrations were used to calculate the pharmacokinetic constants, volume of distribution, elimination rate constant and half-life of the drug. The elimination rate constant (ke) was computed using the following equation: ke=−(InC1-InC2)/(t1-t2). The elimination rate constant was converted into the half-life using: t1/2=0.693/ke. The quotient of the dose and the extrapolated serum concentration at time 02 calculate the hybrid constant volume of distribution/bioavailability (V/F). The extrapolated serum concentration at time zero was calculated using a variant of the intra-venous bolus equation: C=C/e−ket where t and C are the times/concentration pair that occur after administration of the extravascular dose in both the post-absorption and post-distribution phases. Dose proportionality information was obtained by comparing plasma levels of sPIF across all dose levels evaluated across applicable cohorts.

sPIF demonstrated dose-proportional changes in circulating levels of the drug in the range of doses used in this study (0.1, 0.5 and 1.0 mg/kg). The increases in Cmax values were generally greater than dose proportional. The maximal blood level was 9.4 ng/mL. The 0.1 mg/kg sPIF dose did not produce a detectable level and it was <1 ng/ml. Using these parameters, we found that the t1/2 of sPIF following administration was 91 minutes. Similarly, volume of distribution was 22 L using a one compartment model assumption. sPIF plasma level in five patients were detectable >1 ng/ml. Levels peaked between 30 and 60 minutes after the subcutaneous injection. sPIF was not detected in the plasma at four hours after injection.

TABLE 3
Pharmacokinetics of sPIF after a single
subcutaneous dose show rapid clearance

| | sPIF | sPIF | sPIF |
|---|---|---|---|
| | 0.1 mg/kg | 0.5 mg/kg | 1.0 mg/kg |
| PK parameter | (n = 4) | (n = 4) | (n = 4) |
| Mean Cₘₐₓ, ng/mL | NA | 3.7 | 9.4 |
| Mean Tₘₐₓ, min | NA | 30 | 30 |
| Mean T_{1/2}, min | NA | 63 | 109 |
| Mean Cₗₐₛₜ, ng/mL | NA | 0 | 0.5 |
| Mean Tₗₐₛₜ, min | NA | 240 | 240 |

Serial plasma samples were collected from 0-240 minutes and analysed by validated LC/MS/MS method with lowest level of detection set at 1 ng/ml. In both 0.5 and 1 mg/kg but not in the 0/1 mg/kg sPIF dose the pharmacokinetic parameters could be calculated. Cₘₐₓ, maximum concentration; Tₘₐₓ, half-life time taken to reach maximum concentration; T_{1/2}, half-life; Cₗₐₛₜ, last observed quantifiable serum concentration of the drug; Tₗₐₛₜ, time (observed time point) of Cₗₐₛₜ

### Exploratory Analysis—LFTs

The ALT/AST/ALP enzyme levels were measured in chronic AIH patients. AIH is a heterogeneous disease and therefore patients are on steroids and immune suppressive drugs. Some patients, however; remain refractory to treatment.

Patients' serum samples were collected at five different time points; screening, pre-injection at 4 hrs, 24 hrs and on the 8th day post-injection. There was a decrease in ALT and AST to normal levels in two (2) treated patients and maintained until 8 days. Other sPIF-treated patients, saw a transient improvement in their liver indices. In patients with normal liver function, sPIF effect was minimal. One patient's ALP levels reached a normal range. In patients treated with low doses of sPIF (0.1 and 0.5 mg/kg doses), circulating sPIF levels were low, however, there was improvement in both ALT and AST values. (Tables 4, 6, 7)

TABLE 4
Individual LFTs in patients with normal ALT levels
Normal SAD

| Dose | | | | | Dose | | | | | Dose | | | | |
|---|---|---|---|---|---|---|---|---|---|---|---|---|---|---|
| 0.1 | ALT | AST | ALP | Time | 0.1 | ALT | AST | ALP | Time | 0.5 | ALT | AST | ALP | Time |
| | 39 | 26 | 106 | screen | | 23 | 26 | 70 | screen | | 46 | 37.5 | 104.1 | screen |
| | 35.7 | 22.2 | 113.6 | 0 | | | | | 0 | | 44.9 | 47.9 | 104.5 | 0 |
| | 32.4 | 22.2 | 113.6 | 4 hr | | 29.6 | 22 | 72.3 | 4 hr | | 52.4 | 45.6 | 114.8 | 4 hr |
| | 34.5 | 19.2 | 108.3 | 24 hr | | 29 | 24 | 69 | 24 hr | | 38.6 | 41.6 | 108.5 | 24 hr |
| | 33.5 | 23.7 | 108.4 | 192 hr | | 27.7 | 18.9 | 70.6 | 192 hr | | 31 | 34 | 96 | 192 hr |
| Dose | | | | | Dose | | | | | Dose | | | | |
| 0.5 | ALT | AST | ALP | Time | 1.0 | ALT | AST | ALP | Time | 1.0 | ALT | AST | ALP | Time |
| | 34 | 27 | 89 | screen | | 38 | 28 | 89 | screen | | 31 | 30.5 | 127.1 | screen |
| | 40 | 42 | 74 | 0 | | 37 | 27 | 88 | 0 | | 26.4 | 28 | 124.9 | 0 |
| | 41.7 | 38.9 | 70.1 | 4 hr | | 26. | 25. | 73. | 4 hr | | 22.8 | 27.3 | 128 | 4 hr |
| | 42.8 | 35.6 | 69 | 24 hr | | 36 | 25 | 86 | 24 hr | | 23.7 | 28 | 117.9 | 24 hr |
| | 57.5 | 47.2 | 84.8 | 192 hr | | 40. | 29. | 88. | 192 hr | | 28.8 | 29 | 125.5 | 192 hr |

TABLE 6
Individual LFTs in patients with elevated ALT
ABNORMAL SAD

| Dose | | | | | Dose | | | | | Dose | | | | |
|---|---|---|---|---|---|---|---|---|---|---|---|---|---|---|
| 0.1 | ALT | AST | ALP | Time | 0.1 | ALT | AST | ALP | Time | 0.5 | ALT | AST | ALP | Time |
| | 85 | 67.7 | 102 | screen | | 80 | 63 | 84 | screen | | 135 | 100 | 59 | screen |
| | | | | 0 | | 78 | 59 | 83 | 0 | | 127.4 | 82.8 | 59.4 | 0 |
| | 51.2 | 38.1 | 90.5 | 4 hr | | 81 | 61 | 91 | 4 hr | | 103.9 | 70.5 | 46.8 | 4 hr |
| | 49 | 40 | 86 | 24 hr | | 73.9 | 53.3 | 77.3 | 24 hr | | 124 | 84.6 | 57.3 | 24 hr |
| | 42 | 33 | 71 | 192 hr | | 66 | 58 | 82 | 192 hr | | 129.4 | 89 | 55.7 | 192 hr |
| Dose | | | | | Dose | | | | | Dose | | | | |
| 0.5 | ALT | AST | ALP | Time | 1.0 | ALT | AST | ALP | Time | 1.0 | ALT | AST | ALP | Time |
| | 93 | 83 | 75 | screen | | 46.3 | 72.3 | 78.5 | screen | | 52 | 57 | 89 | screen |
| | 69 | 61 | 84 | 0 | | 33 | 43 | 65 | 0 | | 71.2 | 68.2 | 116.2 | 0 |
| | 67.3 | 53.7 | 71.7 | 4 hr | | 25 | 40 | 63 | 4 hr | | | | | 4 hr |
| | 84.3 | 61.1 | 83 | 24 hr | | 30 | 37 | 78 | 24 hr | | 62.8 | 63.1 | 102.6 | 24 hr |
| | 75 | 58 | 95 | 192 hr | | 27 | 33 | 78 | 192 hr | | 59.5 | 57.1 | 96.1 | 192 hr |

TABLE 7
Individual LFTs in patients with normal and elevated ALT levels injected with placebo
Placebo

| ALT | AST | ALP | Time | | ALT | AST | ALP | Time | | ALT | AST | ALP | Time | |
|---|---|---|---|---|---|---|---|---|---|---|---|---|---|---|
| 71.2 | 51.5 | 100.9 | screen | | 58.9 | 39.4 | 118.9 | screen | | 72 | 87 | 110 | screen | |
| | | | 0 | | 26.5 | 78.7 | 74.4 | 0 | | 77 | 88 | 102 | 0 | |
| 49.3 | 29.7 | 113.8 | 4 | hr | 26.5 | 78.7 | 74.4 | 4 | hr | 64 | 95 | 92 | 4 | hr |
| 38 | 27 | 87 | 24 | hr | 43 | 28 | 112 | 24 | hr | 61.8 | 82.7 | 101.3 | 24 | hr |
| 33.5 | 29.5 | 103 | 192 | hr | 40.3 | 26.7 | 109.3 | 192 | hr | 68 | 82 | 99 | 192 | hr |
| 39 | 42 | 92 | screen | | 35.7 | 62.4 | 89.8 | screen | | 93 | 95 | 122 | screen | |
| 37.3 | 34.8 | 82.1 | 0 | | 31.6 | 47.1 | 78.9 | 0 | | 90 | 90 | 106 | 0 | |
| 28.7 | 36 | 86 | 4 | hr | 30.6 | 46.8 | 77 | 4 | hr | | | | 4 | hr |
| 31.4 | 34.2 | 90.6 | 24 | hr | 28 | 49 | 82 | 24 | hr | 77 | 72 | 106 | 24 | hr |
| 31 | 37 | 88 | 192 | hr | 26.6 | 36.8 | 73.8 | 192 | hr | 81 | 70 | 112 | 192 | hr |

### Exploratory Analysis—Cytokines

Serum samples collected were tested for cytokine and chemokine panel (IL-1p IL-4, IL-8, IL-10, IL-17α, IL-17γ, IL-21, IL-22, IL-23, IFNγ, IP-10, MCP-1, TNFα) using MILLIPLEX multiplex magnetic bead panels (EMD Millipore) in the MAGPIX instrument (Luminex Corporation). Median fluorescent intensities (MFI) were analyzed with MILLIPLEX™ Analyst Software (EMD Millipore) and cytokine levels were expressed as pg/mL. The levels pre-dose was compared to levels after 24 hours.

Paired serum samples were available from all 18 patients to study the effect of sPIF on serum cytokines and chemokines. Twenty-four hours post-injection, increased sPIF doses led to changes in cytokines levels. In 7 of the 12 patients with normal or elevated ALT levels, data showed a clear directional change. High concordance for trend either increase or decrease (5 cases) in Th2 (IL 10, IL4) and Th1 cytokines IL8 and IL1b and IFNγ) levels were noted one day after sPIF administration. (Table 8)

TABLE 8
sPIF effect on circulating cytokines after 24 hours

| | | LFTs | IFNg | IL-10 | IL-17a | IL-1b | IL-4 | IL-8 | MCP-1 | TNFa | IL-21 |
|---|---|---|---|---|---|---|---|---|---|---|---|
| PIF | Dose | Nl/Abnl | pg/ml | pg/ml | pg/ml | pg/ml | pg/ml | pg/ml | pg/ml | pg/ml | pg/ml |
| 0 | 0.1 | Normal | A | 29.25 | 5.59 | 16.74 | <0.25↓ | 907.73 | 1045 | 11.99 | 37.41 |
| 24 h | 0.1 | Normal | 7.28 | 32.21 | 5.24 | 1.67 | 29.28 | 121.27 | 798.76 | 5.88 | 76.39 |
| 0 | 0.1 | Normal | 2.41 | 1.11 | 11.53 | 0.98 | 331.08 | 196.58 | 799.58 | 18.62 | 26.46 |
| 24 h | 0.1 | Normal | 1.87 | <0.15↓ | 10.75 | 0.8 | 18.39 | 67.32 | 737.75 | 26.61 | 17.69 |
| 0 | 0.1 | Abnormal | 10.21 | <0.15↓ | 3.47 | 4.02 | 77.77 | 492.86 | 879.94 | 13.46 | 51.1 |
| 24 h | 0.1 | Abnormal | 9.48 | <0.15↓ | 3.64 | 3.46 | 20.51 | 111.77 | 1016 | 14.49 | 65.25 |
| 0 | 0.1 | Abnormal | 1.16 | <0.15↓ | 9.21 | 0.75 | 249.08 | 93.83 | 986.25 | 32.42 | 71.53 |
| 24 h | 0.1 | Abnormal | 0.81 | <0.15↓ | 8.67 | 0.66 | 125.51 | 65.09 | 1059 | 26.29 | 72.57 |
| 0 | 0.5 | Abnormal | 8.38 | 99.3 | 3.29 | 368.8 | 18.39 | >2048↑ | 7155 | 107.44 | 5.19 |
| 24 h | 0.5 | Abnormal | 13.88 | 5172 | 3.64 | >18446↑ | 445.17 | >2048↑ | 8108 | 6225 | <0.98↓ |
| 0 | 1.0 | Normal | 2.94 | 5.48 | <0.06↓ | 0.57 | <0.25↓ | 607.47 | 1823 | 20.69 | 25.73 |
| 24 h | 1.0 | Normal | 4.74 | 14.72 | 1.52 | 59.96 | 2.25 | >2048↑ | 4844 | 63.97 | 13.36 |
| 0 | 1.0 | Normal | 0.12 | <0.15↓ | 2.94 | 1.53 | 22.66 | 691.86 | 912.28 | 5.44 | 20.6 |
| 24 h | 1.0 | Normal | <0.04↓ | <0.15↓ | 1.35 | 0.16 | 8.52 | 279.73 | 669.57 | 3.35 | 34.14 |

### Multiple Ascending Dose (MAD)

Patients 18 to 75 years old of non-child bearing potential with documented autoimmune hepatitis (AIH) and compensated chronic liver disease under standard of care were enrolled in a multiple ascending dose study. The purpose of the study was to establish the safety, tolerability, and pharmacokinetics of sPIF. The sPIF and the placebo groups were similar in terms of demographic and baseline characteristics. The diagnosis of AIH was documented at the screening evaluation by either a pre-treatment score ≥15 or a post-treatment score of ≥17 by the International Criteria for the Diagnosis of Autoimmune Hepatitis. Treatment with oral, immunosuppressive drug(s) was stabilized for at least 6 weeks prior to screening for this study. Permitted concomitant immune suppression medications included azathioprine ≤100 mg per day, budesonide 9≤mg per day, mycophenolate mofetil ≤3000 mg per day, prednisone ≤30 mg per day, ursodeoxycholic acid ≤1500 mg per day, and tacrolimus ≤6 mg per day.

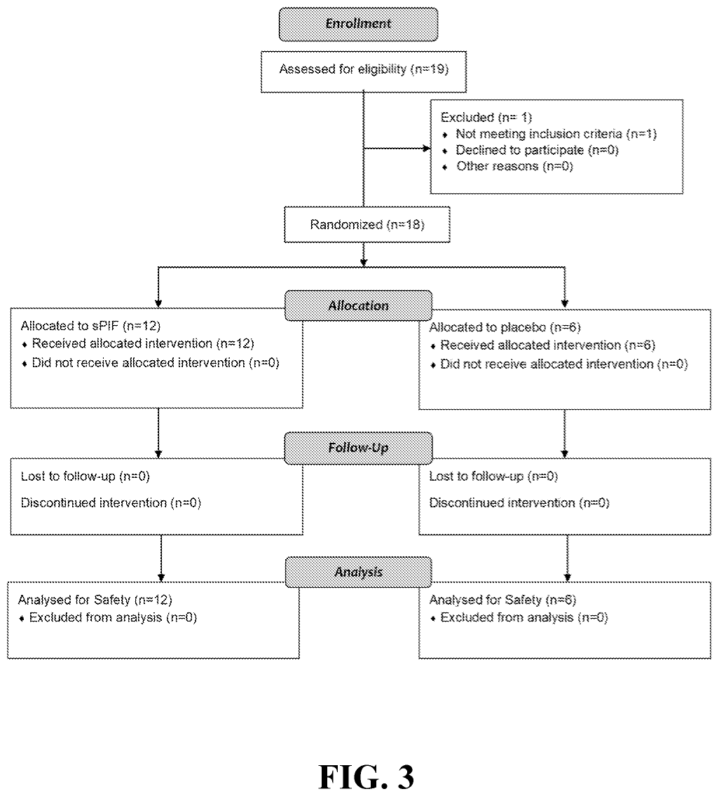

Eighteen (18) patients were enrolled in the study. Twelve (12) patients were randomly assigned to sPIF and 6 to the placebo treatment arm. All enrolled patients completed the trial. (FIG. 3) Patients were enrolled in three cohorts (0.1, 0.5 or 1.0 mg/kg) at a single US site. sPIF was reconstituted from powder to a 0.5 mL total volume per syringe with lactated Ringer's injection, USP (0.1, 0.5, 1 mg/kg). Dose was adjusted to be appropriate for the patient's weight. Lactated Ringer's injection, USP 0.5 mL was used as the placebo and was administered subcutaneously to subjects randomized to receive placebo under the same conditions as the active drug recipients. For sPIF dosing, patients were dosed daily for 5 days. Subsequently, patients were followed at day 8, 15, and 29 of the study.

TABLE 9
Demographics and baseline characteristics

| | placebo | sPIF | sPIF | sPIF | sPIF |
|---|---|---|---|---|---|
| | all | all | 0.1 mg/kg | 0.5 mg/kg | 1.0 mg/kg |
| Female, n (%) | 6 (100%) | 12 (100%) | 4 (100%) | 4 (100%) | 4 (100%) |
| Age (y), mean | 58.83 | 58.00 | 56.75 | 58.75 | 58.50 |
| Age SD | 7.91 | 9.42 | 13.40 | 9.43 | 7.33 |
| Ethnic origin | | | | | |
| Caucasian, n | 4 (66%) | 9 (75%) | 3 (75%) | 4(100%) | 2 (50%) |
| Hispanic, n | 1 (17%) | 3 (25%) | 1 (25%) | 0 (0%) | 2 (50%) |
| Black, n | 1 (17%) | 0 (0%) | 0 (0%) | 0 (0%) | 0 (0%) |
| AIH score, mean +/− SD | 18.0 | 18.9 | 18.8 | 18.5 | 19.5 |
| Pretreatment SD | 0.9 | 1.2 | 1.5 | 1.0 | 1.3 |
| Liver Function Tests | | | | | |
| Total Bilirubin (mg/dL), mean | 0.57 | 0.55 | 0.50 | 0.58 | 0.58 |
| Total Bilirubin SD | 0.25 | 0.19 | 0.18 | 0.29 | 0.13 |
| Albumin (g/dL), mean | 4.02 | 4.08 | 3.95 | 4.35 | 3.95 |
| ALB SD | 0.26 | 0.34 | 0.44 | 0.26 | 0.06 |
| Gamma globulin (U/L), mean | 3.90 | 3.06 | 2.73 | 3.05 | 3.40 |
| Gamma Globulin SD | 1.46 | 0.53 | 0.10 | 0.70 | 0.45 |
| HBsAg (positive), n (%) | 0 (0%) | 0 (0%) | 0 (0%) | 0 (0%) | 0 (0%) |
| Anti-HCV (positive), n (%) | 0 (0%) | 0 (0%) | 0 (0%) | 0 (0%) | 0 (0%) |

### Safety and Tolerability

Safety and tolerability was evaluated by assessment of clinical laboratory tests, periodic physical examination, including vital signs measurements, 12-lead ECG at baseline (pre-dose, Day 1) and at various time points during the treatment phases of the protocol, and by the documentation of adverse events. Concomitant medication intake was also recorded. All adverse events and all treatment related adverse events were listed by subject. Adverse events were summarized by relationship to study drug and severity. In addition to the standard labs, serum cytokines and plasma sPIF were collected.

No grade 2, 3 or 4 adverse events in any of the 12 body systems were reported by symptoms or by physical exam in any patient during the active dosing portion of the study or until day 29 follow-up period. The frequency of the adverse symptom categories was recorded by organ system. One noted SAE was skin irritation at the injection site which resolved within the hour. No grade 2, 3 or 4 adverse events of laboratory values occurred in any patient to date during study drug administration or up to day 29 follow up period. Minor, grade 1 or non-graded (World Health Organization criteria) changes in the blood studies occurred in few patients. The overall adverse events and their frequency by patient number was recorded. There were no clinically significant laboratory results that required study drug to be modified, interrupted, or discontinued. Overall, sPIF's safety was established.

### 1. Pharmacokinetics

Levels of sPIF in plasma pre-injection-baseline and on day 3 and 5 prior to injection were determined to assess sPIF accumulation. Clearance was tested by determining plasma sPIF levels at 3 additional time points until day 29 of the study. sPIF levels were determined by using a validated liquid chromatography and mass spectroscopy (LC-MS/MS) assay (lower limit of quantification 1 ng/mL). sPIF plasma levels were analyzed from baseline pre-injection until day 29 of the study. sPIF levels in all cases even at the 1 mg/kg dose were below <1 ng/ml indicating sPIF clearance is rapid.

### 2. Anti-sPIF Antibody

The method for circulating anti-sPIF antibody detection was reported using a validated assay. Patients' samples from day 0 screening until day 29 of the study were tested. Following sPIF administration, serum samples were tested for anti-sPIF antibodies (9). In all patients (N=18) from prior to injection, serum samples were serially analyzed until day 29 of the study. There was no evidence for an anti-sPIF antibody development. Therefore, sPIF is not immunogenic following repeat administration.

### No Drug to Drug Interaction

The patient population treated in the multiple ascending portion of the study continued under their current standard of care (previously prescribed steroids, immune suppressants and other medications for their co-morbidities). sPIF administration at any dose was not associated with any symptoms or laboratory tests that could indicate drug to drug interaction.

### 3. Exploratory Analysis—Effect on LFTS

ALT/AST/ALP/GGT enzymes levels were evaluated. Patients' serum samples were collected at seven different time points; screening, pre-injection at day 1, day 3, 5 and 8, 15 and 29 post-injection. (Attachment 2, Tables 2-4)

AIH is a heterogenous condition of unknown and diverse etiology, hence one or more LFTs can be affected. ALT/AST reflect mainly hepatocyte function while ALP/GGT levels reflect biliary duct function. sPIF was administered to chronic AIH patients with both normal and in some cases, abnormal LFTs. Despite steroid and immune suppressive use, some patients remained refractory to treatment.

Overall, a trend in all 4 enzymes towards improved LFTs levels in sPIF treated patients was observed. Patients with normal LFTs maintained normal range enzyme levels while a majority of those with abnormal enzyme levels showed improved levels. Though sPIF dosing was 5 days, the improvement in enzyme levels in some cases lasted one week and even up to day 29 of the study. In 2 patients, ALT levels returned to the normal range. In 1 patient AST and ALP levels reached the normal range. In patients where ALT/AST levels were in the normal range, sPIF also reduced ALP levels to normal in 2 patients and GTT levels two-fold in 1 patient reflecting an integrated effect on the liver. Ten out of twelve patients showed improvement in at least one and up to four of their liver enzymes improve.

Normal MAD

| Dose | Time | | | | | Dose | | | | | Dose | | | | |
|---|---|---|---|---|---|---|---|---|---|---|---|---|---|---|---|
| 0.1 | (days) | AST | ALT | AP | GGT | 0.1 | AST | ALT | AP | GGT | 0.5 | AST | ALT | AP | GGT |
| | Screen | 16 | 14 | 98 | | scr | 13 | 10 | 70 | | | 23 | 23 | 88 | |
| | 0 | 22 | 28 | 117 | <10 | 0 | 21 | 24 | 93 | 20 | | 32 | 33 | 100 | 17 |
| | 5 | 23 | 32 | 112 | 10 | 3 | 20.6 | 23.6 | 86.4 | 21 | | 27 | 35 | 96 | 14 |
| | 8 | 22 | 31 | 109 | <10 | 3 | 21 | 22 | 84 | 20 | | 33 | 42 | 103 | 16 |
| | 15 | 21.2 | 24.8 | 104.7 | <10 | 3 | 23 | 24 | 86 | 21 | | 35 | 39 | 94 | 16 |
| | 29 | 22 | 28 | 111 | <10 | 15 | 31 | 26 | 82 | 19 | | 29.1 | 24.8 | 90.1 | 13 |
| | | | | | | 29 | 21.5 | 26.1 | 85.2 | 19 | | 35 | 39 | 109 | 15 |
| Dose | Time | | | | | Dose | | | | | Dose | | | | |
| 0.5 | (days) | AST | ALT | AP | GGT | 1.0 | AST | ALT | AP | GGT | 1.0 | AST | ALT | AP | GGT |
| | screen | 20 | 20 | 71 | | | 39.2 | 49.5 | 120.6 | 41 | | 28 | 27 | 141 | |
| | 0 | 24 | 33.2 | 65.5 | 31 | | 43 | 52 | 126 | 42 | | 29.7 | 29.1 | 134.3 | 187 |
| | 3 | 27 | 30 | 61 | 27 | | 43.9 | 62.7 | 122.7 | 41 | | 28 | 33 | 129 | 195 |
| | 5 | 26 | 31 | 69 | 31 | | 45.8 | 51.8 | 127 | 42 | | 28.6 | 24.7 | 128.2 | 196 |
| | 8 | 23 | 25 | 69 | 30 | | 57 | 61 | 150 | 50 | | 31 | 24.1 | 115 | 147 |
| | 15 | 24.5 | 20.9 | 71.9 | 30 | | 48 | 54 | 109 | 39 | | 29 | 33 | 104 | 111 |
| | 29 | 27.1 | 33.5 | 69.9 | 32 | | 54 | 67 | 113 | 48 | | 27.4 | 24.5 | 121.2 | 92 |

Upper limit Normal ALT = 69 Liver cell (hepatocyte damage)
Upper limit Normal AST = 46 Liver cell (hepatocyte damage)
Upper limit Normal = ALP 98: Bile ducts disease confirm when GGT high
Upper limit GGT = 51-The GGT test can diagnose liver damage, specifically bile ducts damage or obstruction. Relevant when combined with high ALT.

ABNORM AL MAD

| | Time | | | | | Dose | | | | | Dose | | | | |
|---|---|---|---|---|---|---|---|---|---|---|---|---|---|---|---|
| Dose 0.1 | (days) | AST | ALT | AP | GGT | 0.1 | AST | ALT | AP | GGT | 0.5 | AST | ALT | AP | GGT |
| | screen | 70 | 81 | 112 | 154 | | 88 | 134 | 50 | | | 68 | 42 | 146 | |
| | 0 | 72 | 62 | 116 | 159 | | 106 | 166 | 52 | 21 | | 58 | 39 | 112 | 173 |
| | 3 | 74.9 | 68.7 | 116.4 | 158 | | 112.6 | 186.2 | 58 | 22 | | 63.5 | 42 | 124.1 | 172 |
| | 5 | 64.6 | 73.9 | 114.9 | 161 | | 119 | 176 | 59 | 22 | | 61 | 48 | 110 | 162 |
| | 8 | 69 | 68.5 | 151.1 | 188 | | 114.5 | 194.4 | 53.4 | 22 | | 62 | 40 | 122 | 163 |
| | 15 | 107.3 | 107.6 | 153.1 | 222 | | 129 | 187 | 56 | 25 | | 61.7 | 31.8 | 116.1 | 164 |
| | 29 | 74.3 | 84.2 | 125.5 | 194 | | 157 | 233 | 53 | 25 | | 63.3 | 46.4 | 125.6 | 150 |
| | Time | | | | | Dose | | | | | Dose | | | | |
| Dose 0.5 | (days) | AST | ALT | AP | GGT | 1.0 | AST | ALT | AP | GGT | 1.0 | AST | ALT | AP | GGT |
| | screen | 50 | 57 | 87 | | | 101 | 121 | 100 | | | 39.1 | 52.9 | 129.5 | 44 |
| | 0 | 58 | 77 | 102 | 281 | | 108.6 | 137.3 | 80.9 | 155 | | 23.2 | 41.7 | 128.2 | 39 |
| | 3 | 54 | 69 | 90 | 251 | | 103.5 | 128.9 | 90.3 | 164 | | 25.1 | 39.9 | 125.4 | 39 |
| | 5 | 57 | 74 | 97 | 252 | | 98.1 | 123.4 | 85.3 | 157 | | 25.1 | 40.5 | 131.3 | 40 |
| | 8 | 53 | 68 | 90 | 241 | | 114 | 134 | 106 | 156 | | 27.7 | 39 | 120.3 | 35 |
| | 15 | 47 | 59 | 88 | 226 | | 110.5 | 148.5 | 78.2 | 137 | | 37.3 | 48.5 | 125.7 | 40 |
| | 29 | 49.9 | 67.6 | 82.5 | 192 | | 117 | 154 | 86 | 138 | | 38 | 57 | 127 | 50 |

Placebo Normal

| Time | | | | | | | | | | | | |
|---|---|---|---|---|---|---|---|---|---|---|---|---|
| (days) | AST | ALT | AP | GGT | AST | ALT | AP | GGT | AST | ALT | AP | GGT |
| screen | 16 | 19 | 87 | | 17 | 11 | 69 | | 27.2 | 39 | 78.8 | 29 |
| 0 | 28 | 33 | 97 | 33 | 25 | 19 | 68 | | 26.5 | 41.5 | 83.7 | 30 |
| 3 | 27.8 | 36.8 | 91.3 | 30 | 25 | 23 | 67 | <10 | 26.9 | 41.8 | 85.8 | 30 |
| 5 | 29.8 | 43.2 | 87.5 | 29 | 20 | 22 | 63 | <10 | 29.1 | 31 | 75.8 | 27 |
| 8 | 23 | 27.2 | 86.1 | 27 | 22 | 25 | 62 | <10 | 30 | 34 | 91 | 30 |
| 15 | 27 | 41 | 99 | 31 | 24 | 24 | 65 | <10 | 27 | 32 | 80 | 28 |
| 29 | 32.5 | 59.8 | 101.3 | 30 | 24.6 | 28.6 | 74.8 | <10 | 26 | 41 | 85 | 36 |

Placebo Abnormal

| Time | | | | | | | | | | | | |
|---|---|---|---|---|---|---|---|---|---|---|---|---|
| (days) | AST | ALT | AP | GGT | AST | ALT | AP | GGT | AST | ALT | AP | GGT |
| screen | 41 | 39 | 106 | | 72 | 114 | 112 | 26 | 82 | 39 | 89 | |
| 0 | 44.1 | 37.2 | 129.2 | 51 | 57 | 118 | 104 | 24 | 74 | 41 | 95 | 26 |
| 3 | 48 | 38 | 132 | 50 | 55 | 115 | 98 | 21 | 65 | 41 | 89 | 25 |
| 5 | 52 | 41 | 119 | 50 | 56 | 113 | 107 | 23 | 64 | 39 | 87 | 25 |
| 8 | 52 | 48 | 127 | 51 | 52.7 | 113.6 | 105.5 | 22 | 60 | 36 | 76 | 23 |
| 15 | 42.7 | 38.2 | 120 | 45 | 51 | 102 | 98 | 22 | 62.3 | 26.4 | 79.8 | 24 |
| 29 | 36 | 27 | 128 | 47 | 56 | 90 | 88 | 19 | 64.6 | 41.1 | 82.5 | 26 |

### 4. Exploratory Analysis—Cytokines

Cytokines IL-1p, IL-4, IL-8, IL-10, IL-17A, IL-21, IFNγ, IP-10, MCP-1, TNFα levels were determined in undiluted serum using MILLIPLEX multiplex cytokine magnetic bead panels (EMD Millipore) in the MAGPIX instrument (Luminex Corporation). Median fluorescent intensities (MFI) were analyzed with MILLIPLEX™ Analyst Software (EMD Millipore) and cytokine levels expressed as pg/ml.

The effect of sPIF on both Th1 and Th2 cytokines was examined serially from baseline until day 8 of the study. In 6 patients, although baseline IL8 levels were different, sPIF regulated this inflammatory cytokine. IL8 is recognized as an important AIH marker. In addition, in 4 patients, a similar trend on Th1 (IL1b, IL17a) cytokine levels was noted. (Attachment 2, Table 5)

sPIF Effect on Circulating Cytokines Serially Tested

| | | IFNg | IL-10 | IL-17a | IL-1b | IL-8 | IP-10 | MCP-1 | TNFa |
|---|---|---|---|---|---|---|---|---|---|
| S. No. | Time | pg/ml | pg/ml | pg/ml | pg/ml | pg/ml | pg/ml | pg/ml | pg/ml |
| 0.1 | Day 0 | 1.21 | <3.39↓ | <0.80↓ | 16.03 | 262.3 | 1560 | 434.13 | 15.15 |
| | Day 3 | 1.21 | <3.39↓ | <0.80↓ | 28.96 | 488.13 | 1735 | 409.98 | 16.89 |
| | Day 5 | 1.46 | <3.39↓ | <0.80↓ | 4.18 | 672.27 | 1611 | 439.48 | 16.02 |
| | Day 8 | 1.21 | <3.39↓ | <0.80↓ | 25.91 | 870.87 | 1741 | 498.73 | 17.11 |
| 0.1 | Day 0 | 2 | 3.83 | 0.96 | 5.63 | 1210 | 870.7 | 416 | 11.72 |
| | Day 3 | 8.24 | 16.46 | 1.2 | 2.77 | 1366 | 1669 | 560.79 | 13 |
| | Day 5 | 4.39 | 9.15 | 1.45 | 5.02 | 1817 | 950.85 | 374.58 | 14.72 |
| | Day 8 | 2.62 | 4.16 | 1.59 | 8.56 | 1453 | 690.6 | 471.28 | 13 |
| 0.5 | Day 0 | 4.78 | <3.39↓ | 2.01 | 2.43 | 4030 | 447.84 | 1181 | 18.63 |
| | Day 3 | 4.78 | 3.83 | 2.01 | 5.87 | 6619 | 430.93 | 1662 | 22.8 |
| | Day 5 | 4.78 | 4.86 | 2.16 | 3 | 6024 | 365.27 | 1538 | 18.63 |
| | Day 8 | 5.18 | 3.67 | 1.87 | 2.65 | 14129 | 448.38 | 1616 | 19.51 |
| 0.5 | Day 0 | 1.72 | 18.57 | 1.2 | 22.5 | 8704 | 1213 | 1351 | 14.29 |
| | Day 3 | 1.21 | 13.78 | 0.84 | 18.29 | 652.84 | 828.37 | 1104 | 11.51 |
| | Day 5 | <1.05↓ | 14.84 | 0.84 | 0.93 | 223.63 | 1116 | 1327 | 10.87 |
| | Day 8 | 1.46 | 18 | 1.07 | 2.54 | 715.04 | 983.28 | 1424 | 9.82 |
| 1 | Day 0 | 13.7 | 11.51 | 3.62 | 2.09 | 399.23 | 1159 | 1410 | 17.32 |
| | Day 3 | 38.25 | 10.54 | 8.22 | 3.47 | 198.11 | 1627 | 1558 | 24.78 |
| | Day 5 | 38.87 | 11.88 | 8.22 | 2.43 | 269.81 | 1553 | 1572 | 22.8 |
| | Day 8 | 51.59 | 11.75 | 11.18 | 2.31 | 86.85 | 1556 | 1626 | 20.82 |
| 1 | Day 0 | 3.29 | <3.39↓ | <0.80↓ | 1.87 | 1755 | 557.89 | 1274 | 21.15 |
| | Day 3 | 12.14 | <3.39↓ | 1.2 | 3.47 | 6008 | 378.94 | 1273 | 20.82 |
| | Day 5 | 20.83 | <3.39↓ | 1.45 | 1.81 | 489.53 | 437.53 | 1922 | 27.86 |
| | Day 8 | 4.39 | <3.39↓ | <0.80↓ | 1.43 | 290.84 | 380.22 | 1532 | 18.2 |

Therefore testing of sPIF in chronic demonstrated safety, no drug to drug interaction, no anti-PIF antibody development. Rapid sPIF clearance. In addition, exploratory studies showed improved LFTS sometime lasting close to 4 weeks post-treatment. The was also a trend in in changes in systemic inflammatory cytokines and chemokines. Thus sPIF may prevent progress to more advanced disease such as NAFLD.

### Example 2

sPIF Hepatitis C Induced Human Hepatocytes Inflammation—Risk Factors for NASH

Currently, effective immunomodulatory agents, with minimal side effects, that can regulate inflammation in the liver to promote liver health in the setting of chronic liver disease have not been developed and they are desperately needed. PIF has already been demonstrated to be safe for patients with liver disease from the ongoing Phase I/II studies and data has been generated to demonstrate a beneficial effect on inflammatory responses both on the liver and circulating cytokines. Specifically, our study was focused on understanding the potential benefits of sPIF for patients with inflammatory liver diseases by specifically focusing on inflammatory responses activated in hepatitis C (HCV) stimulated primary human hepatocytes (PHH, and HepaRG). In addition, the effect of TxpIC on these cells were tested This is a potent chemical mimicking viarl infection in the liver. As new sPIF mutants were developed (N=10) by modifying a single amino acid (Attachment 3) and examining the decrease in K+ flux as compared with wild type sPIF. The data showed that both mutant 3 and 10 have shown increased K+ inhibition as compared with sPIF in a dose comparison analysis. There fore these mutants were tested on human primary hepatocytes comparing with the effect of sPIF in side by side studies.

| | | Kv flux 100% in nt | |
|---|---|---|---|
| Results: conc. 0.625 uM | | (stimulus buffer only) | Effectiveness |
| PIF | MVRIKPGSANKPSDD | 66.22446618 | 33.77553382 |
| PIF-pos1 | MVRIWPGSANKPSDD | 52.20411304 | 47.79588696 |
| PIF-pos2 | MVRIFPGSANKPSDD | 117.017623 | −17.01762297 |
| PIF-pos3 | MVRIYPGSANKPSDD | 47.94738026 | 52.05261974 |
| PIF-pos4 | MVRIIPGSANKPSDD | 101.1172803 | −1.117280267 |
| PIF-pos5 | MVRIKPYSANKPSDD | 77.18982925 | 22.81017075 |
| PIF-pos6 | MVRIKPWSANKPSDD | 120.4525042 | −20.4525042 |
| PIF-pos7 | MVRIKPGSWNKPSDD | 122.2673293 | −22.26732929 |
| PIF-pos8 | MVRIKPGSANWPSDD | 54.21251304 | 45.78748696 |
| PIF-pos9 | MVRIKPGSANKWSDD | 65.9072517 | 34.0927483 |
| PIF-pos10 | MVRIKPGSANKPSFD | 46.28104675 | 53.71895325 |

Methods Primary human hepatocytes (PHHs) and HepaRG™ cells were studied. The later are terminally differentiated hepatic cells derived from a human hepatic progenitor cell line that retains many characteristics of primary human hepatocytes were purchased from Thermo Fisher Scientific Inc. (MA, USA). PHHs were also obtained from Dr. David Geller, at the University of Pittsburgh, through the NIH funded Liver Tissue and Cell Distribution System (LTCDS) and maintained in Williams E Medium containing cell maintenance supplement reagents from Invitrogen (Carlsbad, Calif.). The HCV JFH strain was used in cell culture experiments at an M.O.I. of 0.5-1. The cells treated with JFH1 virus for 0-72 h h with PIF added at concentrations of 20 to 2000 nM at the time of infection. In other experiments TxpIC at 500 ng/ml were added to the cells. This chemical replicates a viral induced inflammation and therefore it can be used in different cell types also when hepatitis C infection is difficult to induce. In PHHs with TxpIC added for 18 hours and measured various cytokine/chemokine levels in culture supernatants lead to 100 fold increase in RANTES, MIP-1a, MIP-1p, IP-10, and IL-6 secretion. Li, K. (2012). Hepatology, 55: 666-675. For analysis of endogenous mRNA levels, total RNA was isolated from cells using the RNeasy RNA extraction kit (Qiagen, Valencia, Calif.) and cDNA synthesis was performed using 0.5 μg of total RNA (Transcriptor First Strand cDNA Synthesis Kit, Roche, Basel, Switzerland). Fluorescence real-time PCR analysis was preformed using an ABI 7500 instrument (Applied Biosystems, Foster City, Calif.) and TaqMan gene expression assay (Applied Biosystems). Relative amounts of mRNA, determined using a FAM-Labeled TaqMan Probe, were normalized to the 18S ribosomal RNA levels in each PCR reaction using the eukaryotic 18S rRNA endogenous control from ABI (VIC/MGB Probe, Primer Limited, Cat #4319413E). The delta 2(-Delta Delta C(T)) method was used for quantitation of relative mRNA levels and fold induction.

### Results:

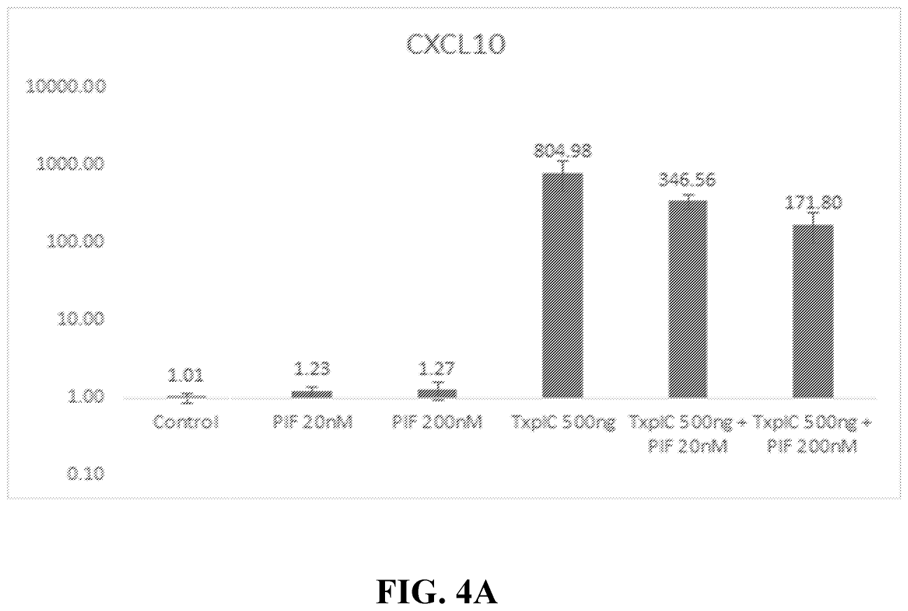

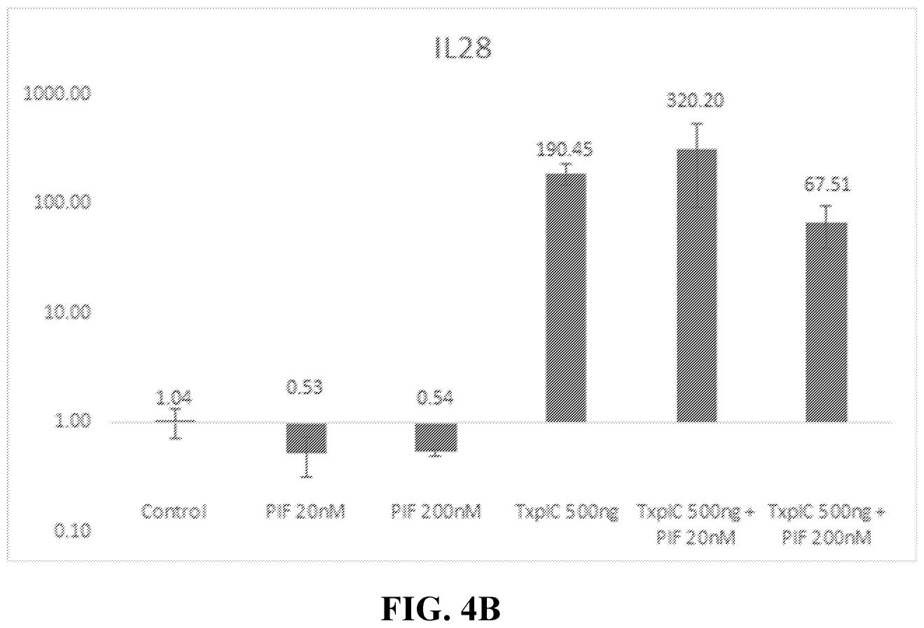

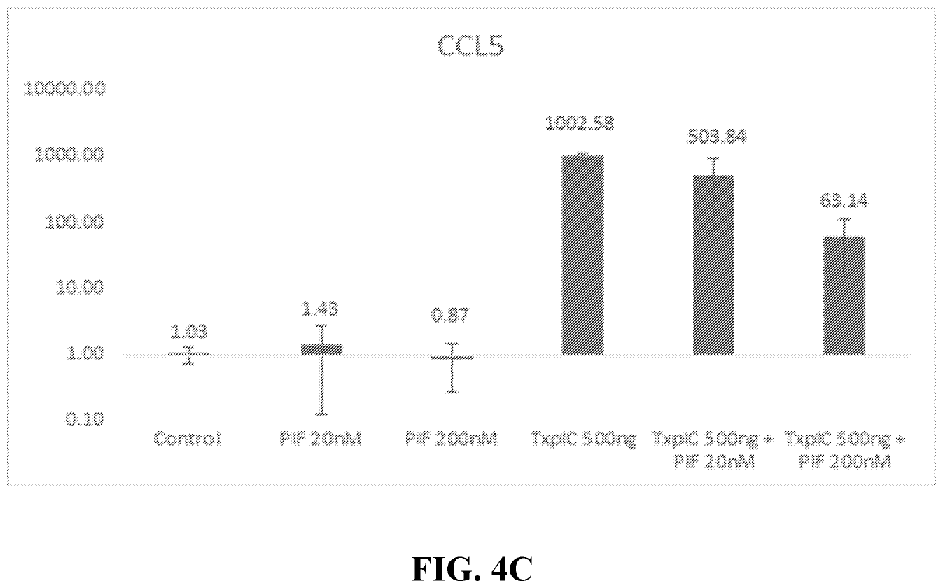

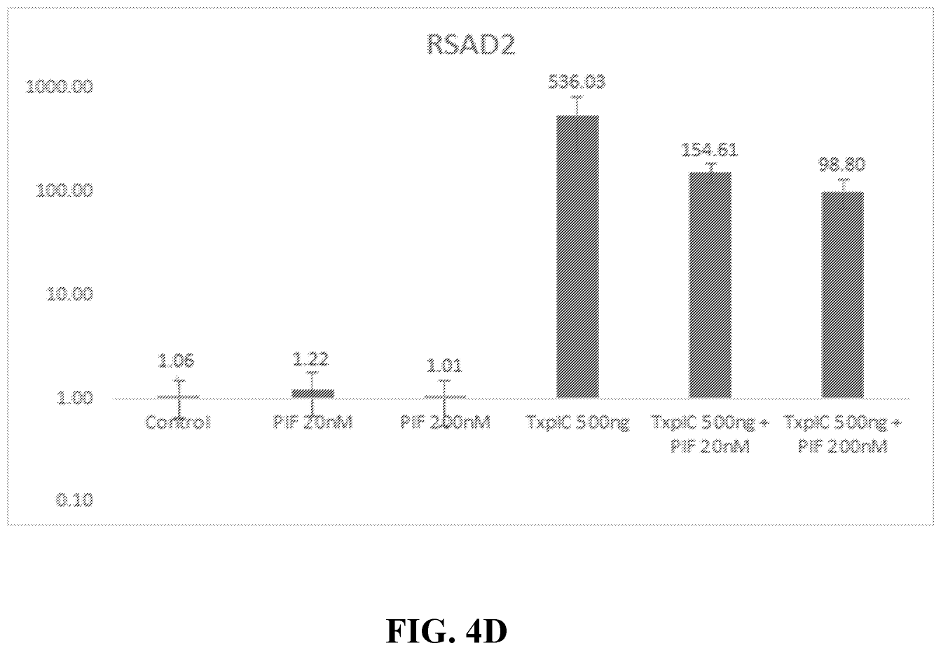

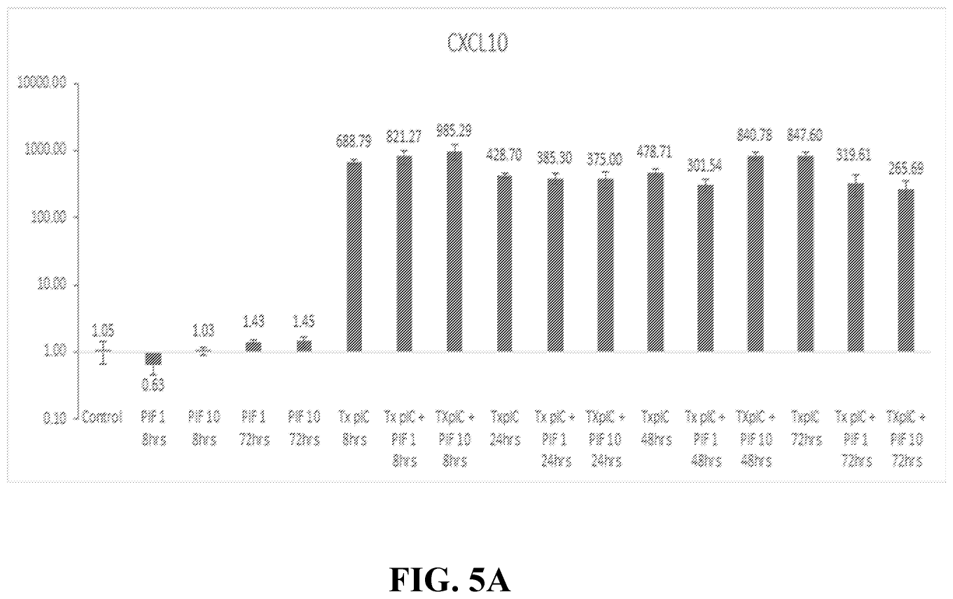

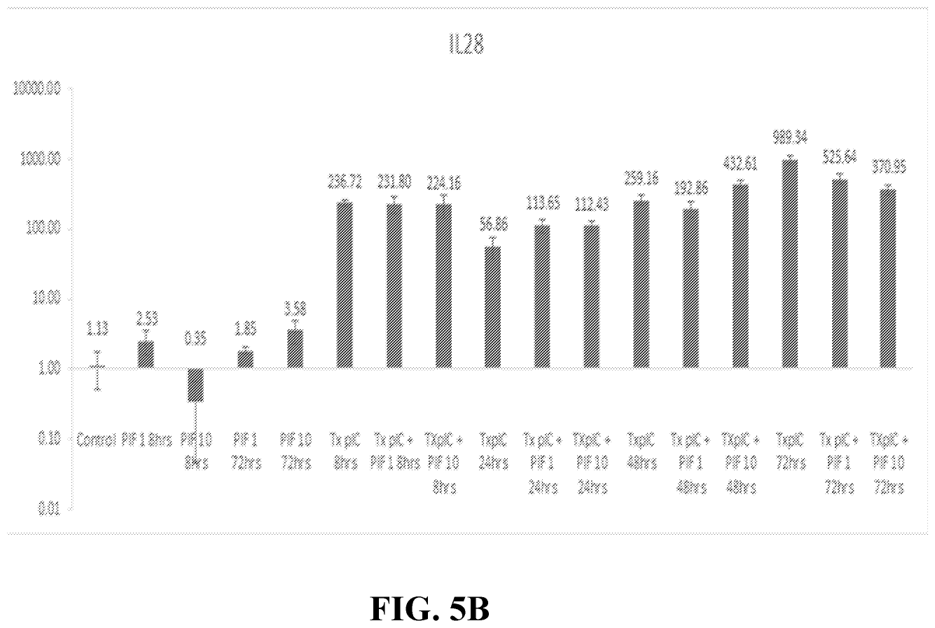

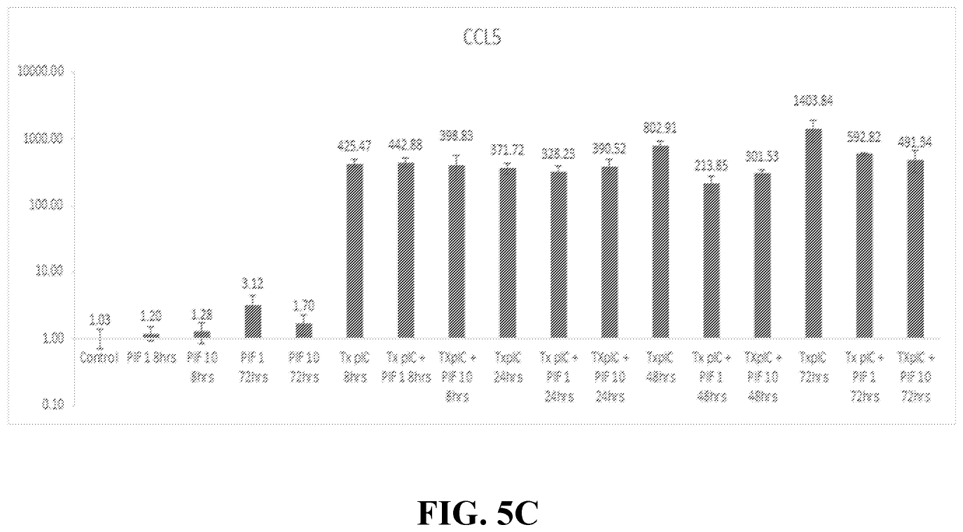

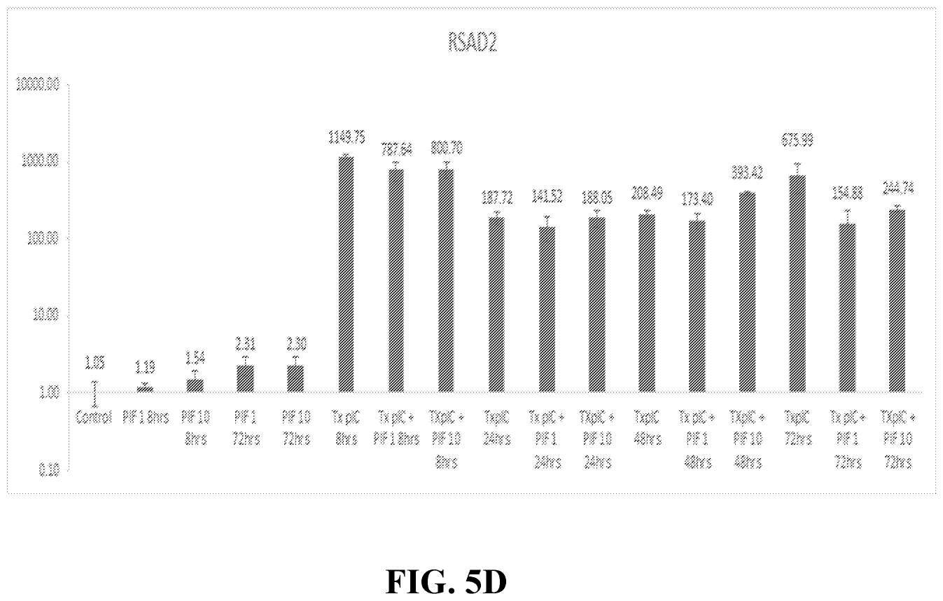

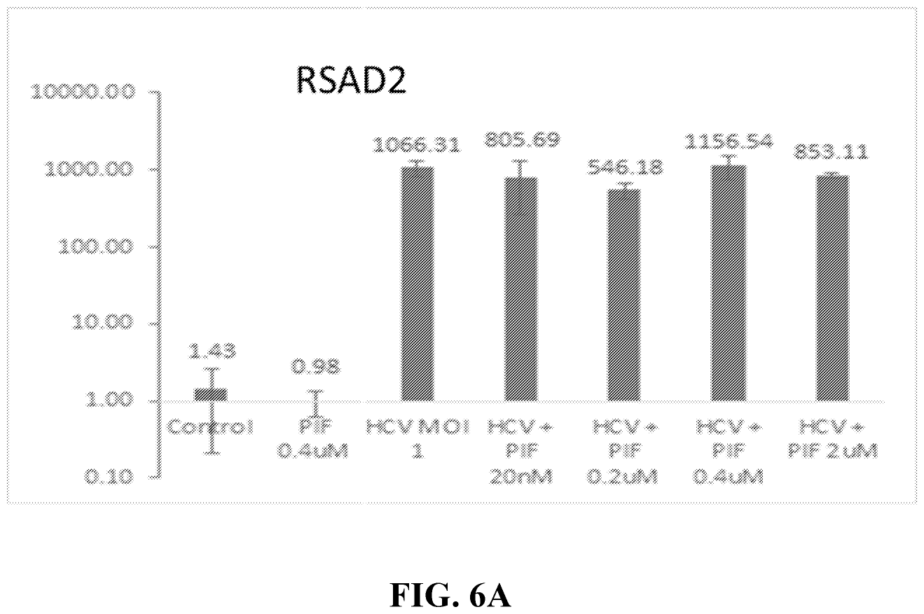

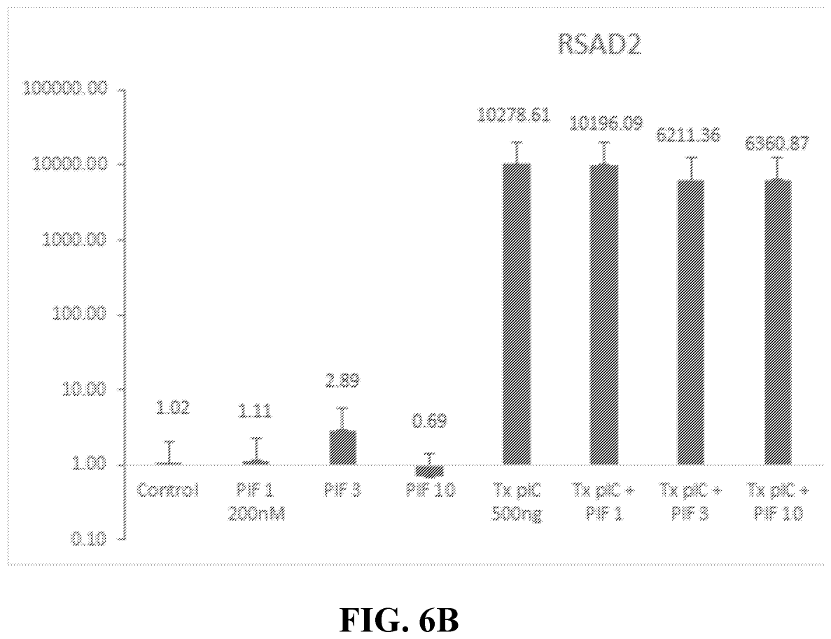

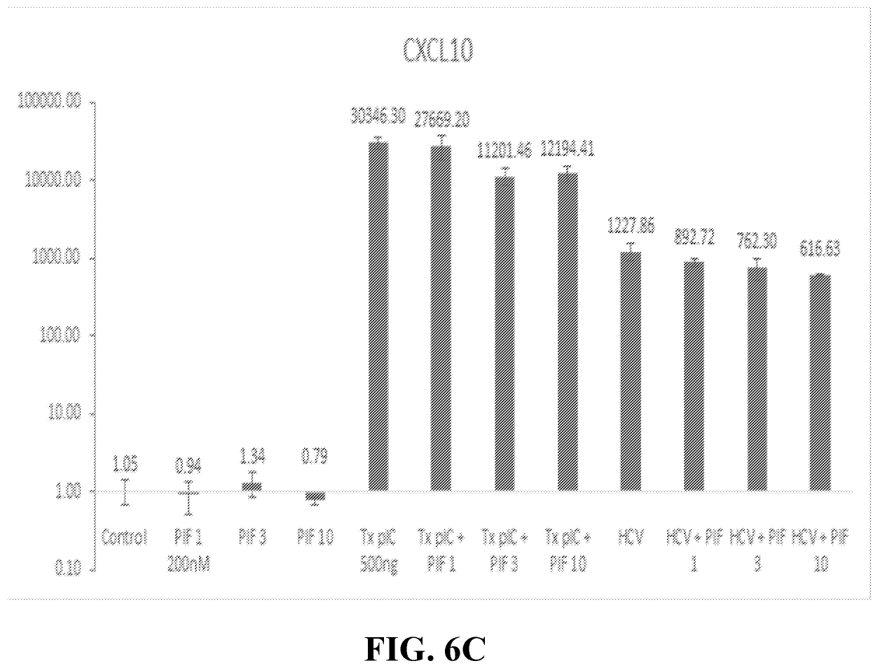

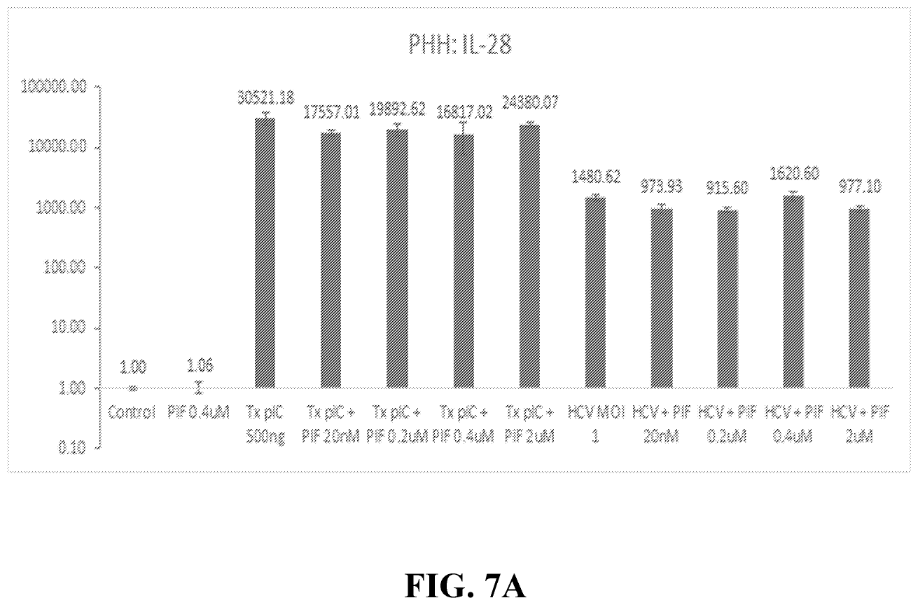

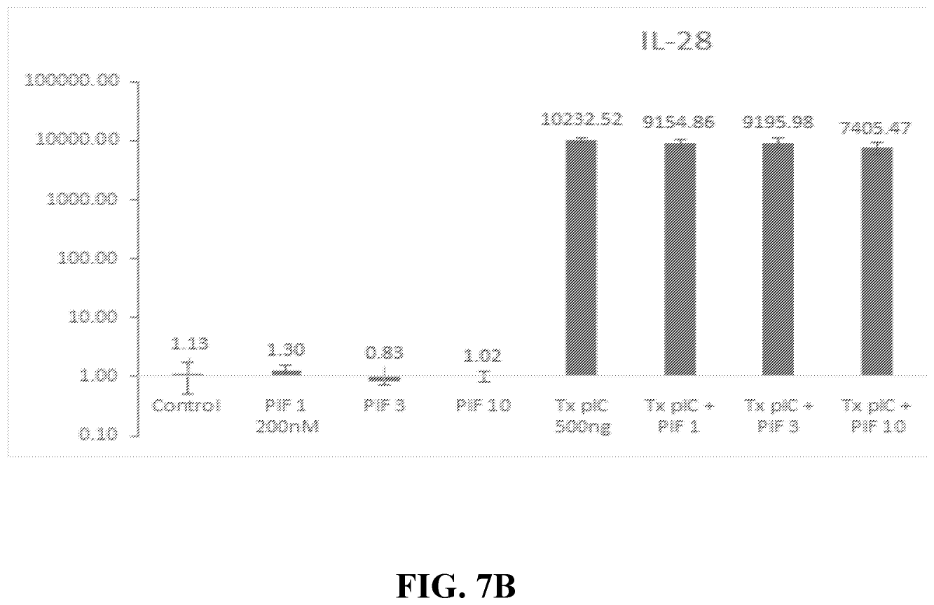

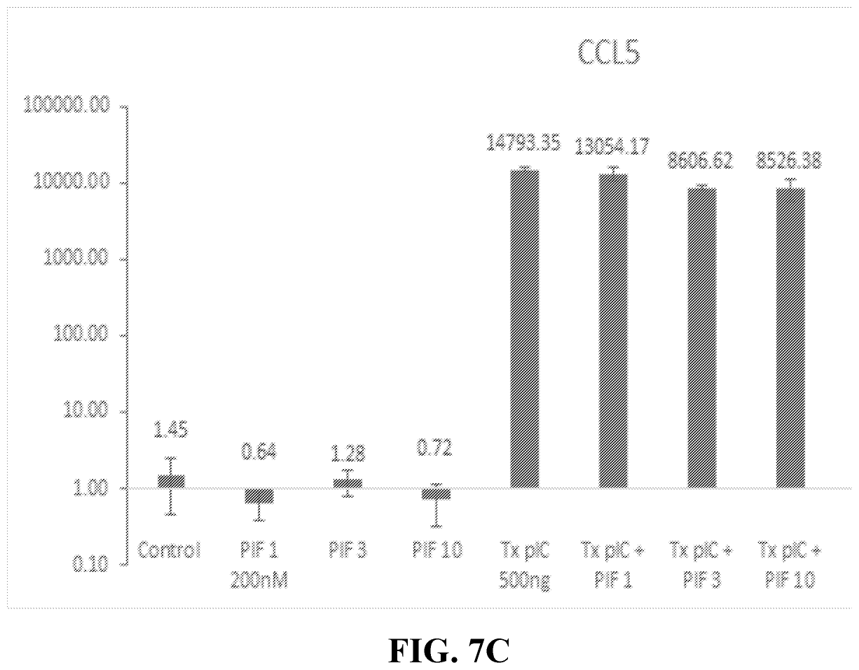

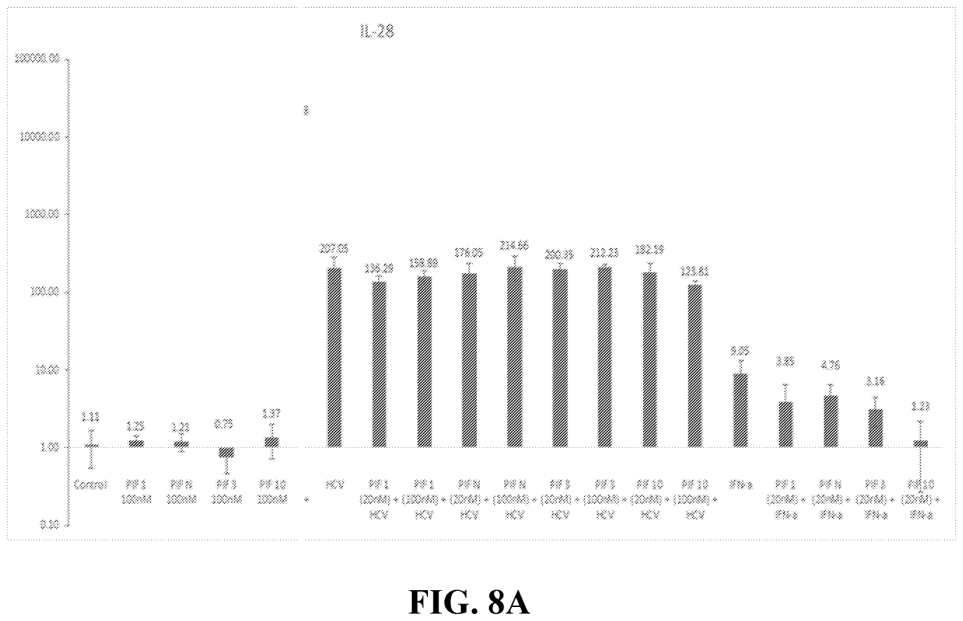

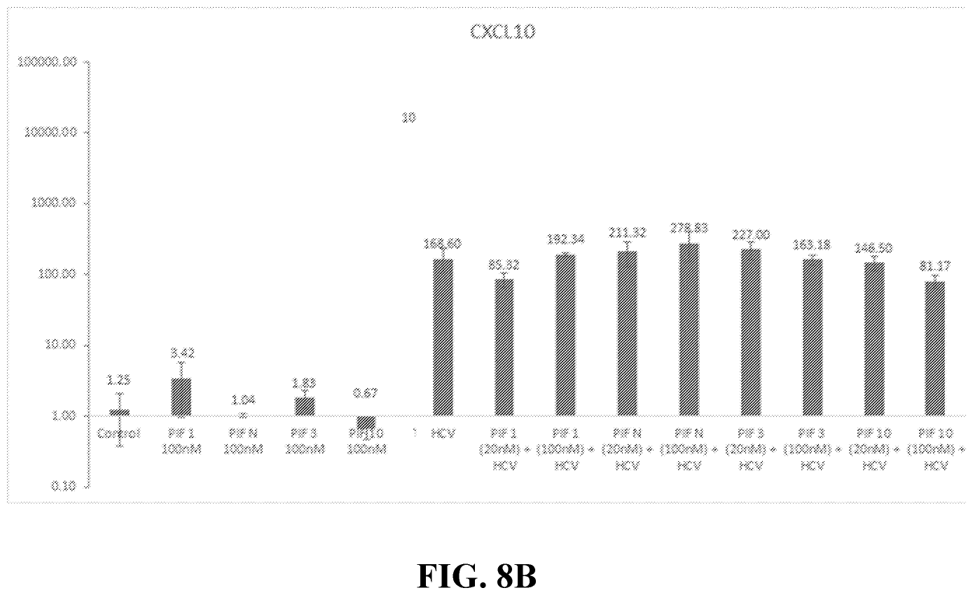

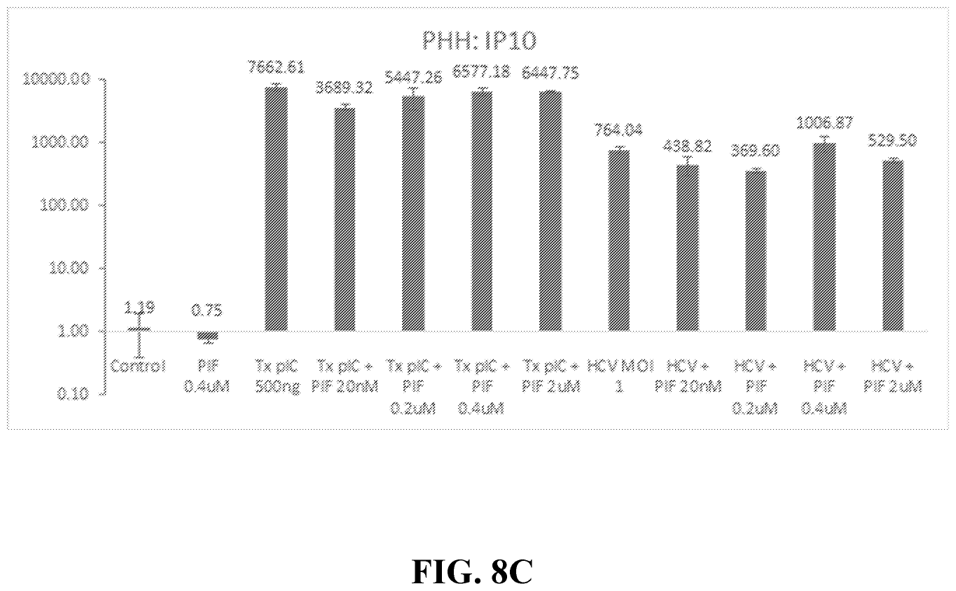

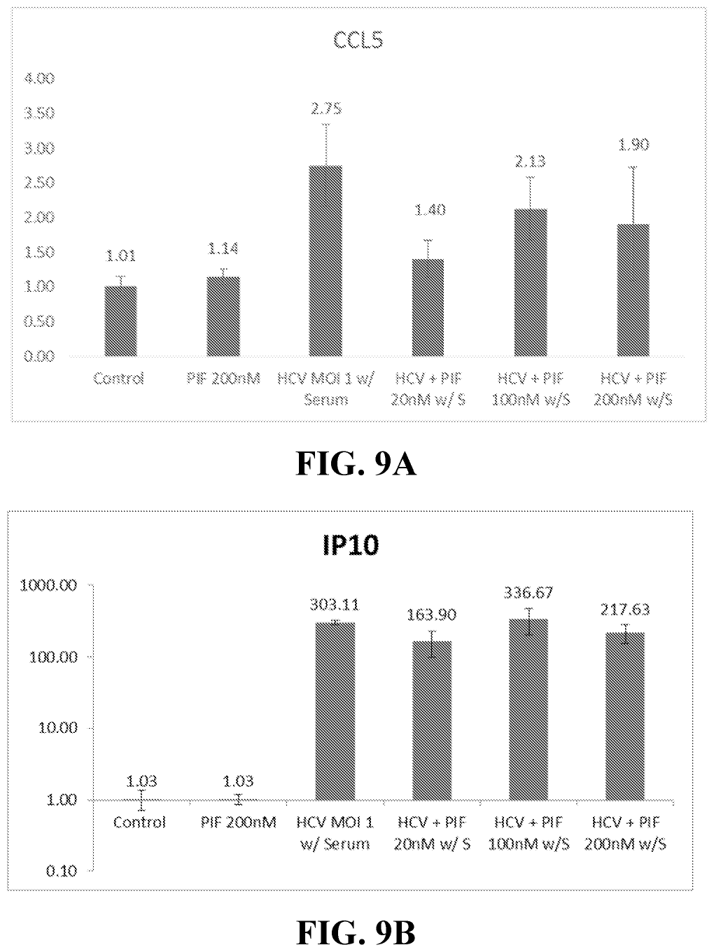

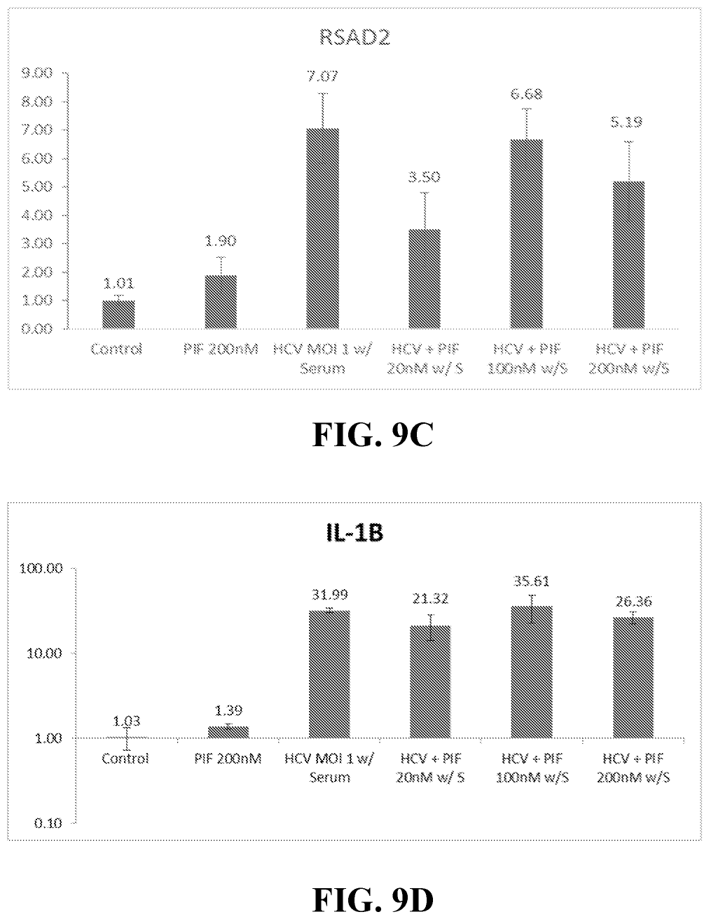

FIG. 4: The effect of TxpIC 500 ng/ml on HepaRg was tested using sPIF. A dose dependent decrease was observed in CXCL10, CCL5, IL28 and RSAD2 expression. Maximal effect was shown at 200 nM sPIF.
FIG. 5 The anti-inflammatory effect of mutant 10 (See table for lists) was compared with sPIF. The data showed the time dependent effect of sPIF and Mutant 10sPIF in culture HepaRG induced by TxpIC 500 ng/ml. The mutant (sPIF 10) led to a more pronounced suppression than sPIF. This was shown with CxCL10, IL28, CCL5. The effect was similar with RSAD2.
FIG. 6. A.sPIF dose dependent inhibitory effect of sPIF on RSAD2 in PHH. The maximal inhibitory effect on HCV infection was present at 200 nM. The effect on TxpIC induced inflammation was already at 20 nM. B. sPIF 200 nM and Mutant 3 and 10 inhibit Tx pIC induced RSAD2. C. As compared with sPIF Mutant 3 sPIF and 10 inhibit Tx pIC and HCV induced CxCI10 expression.
FIG. 7A. sPIF has a dose dependent inhibitory effect on IL28 induced by 500 ng TxpIC or by HCV in PHH. B. Inhibitory effect of sPIF Mutant 10 on HCV induced IL28. C. Inhibitory effect of mutant 3 and 10 on 500 ng/ml TpxIC induced CCL5.
FIG. 8A. Inhibitory effect of sPIF and Mutant 10 (20 nM) on HCV induced IL28 expression by PHH. sPIF, Mutant 3 and 10 potentiate INFa induced suppression of HCV. B. sPIF and mutant 10 inhibit HCV induced CXCL10. C. sPIF inhibits IP10 by TxpIC and induced by HCV in dose dependent manner. Maximal effect 20 nM and 200 nM, respectively.
FIG. 9. sPIF inhibits HCV induced IP10 (A), RSAD2, (B) and CCL5 (C) in THP1 cells. Mild inhibitory effect on IL1B.

The data generated using sPIF and mutant 10 shows that they are effective in reducing prime HCV and TpxIC induced increase in inflammatory cytokines/chemokines hall mark of viral infection. Importantly both sPIF and mutant 10 were effective both in PHH—primary human liver cells and HepaRg—that have similar characteristics as PHH. Recognizing that HCV is a risk factor for NASH such data support sPIF potential protective role.

### Example 3

sPIF Protection Against CMV Infection-Risk Factor for NAFLD

Viral infection contribute to NAFLD and may lead to NASH. We have examined whether the CMV virus can be treated using sPIF as monotherapy, which beyond the liver and spleen infection where a high viral load is present leads to weight loss it also affects the brain.

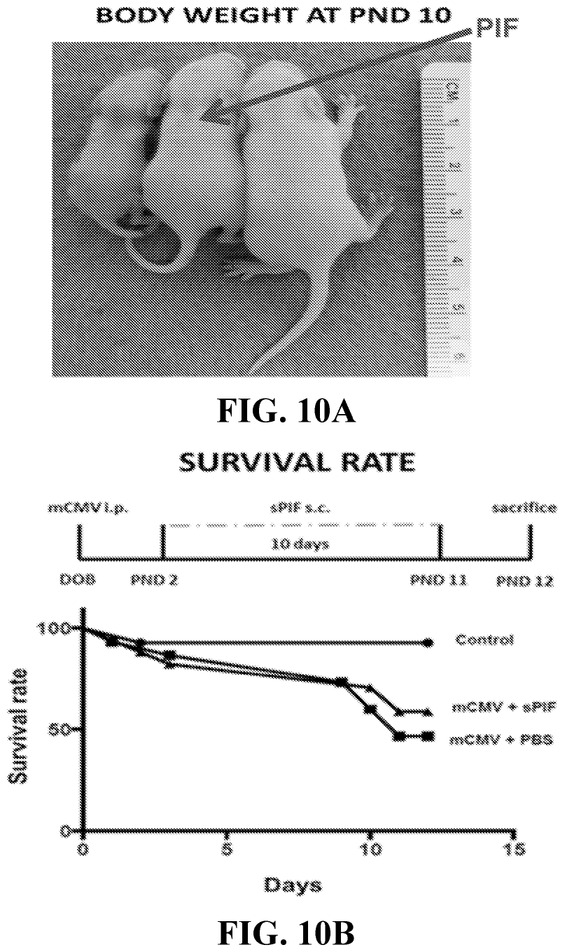

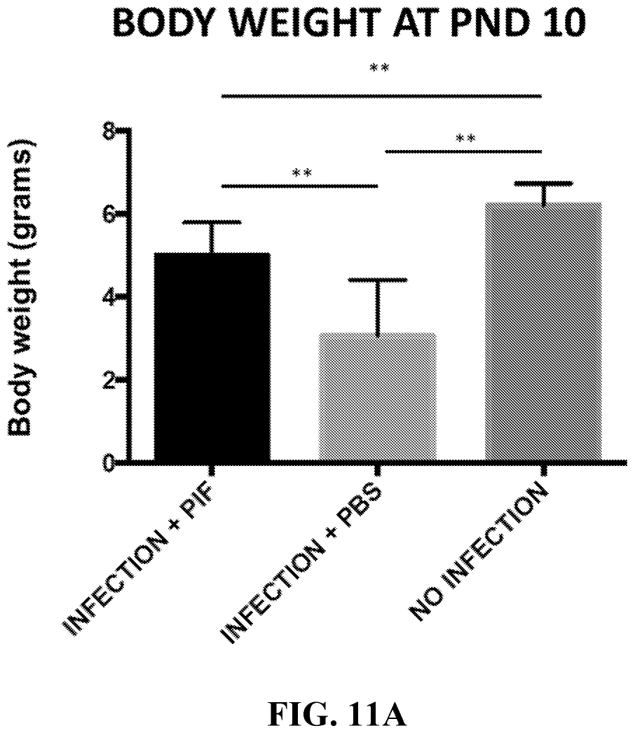

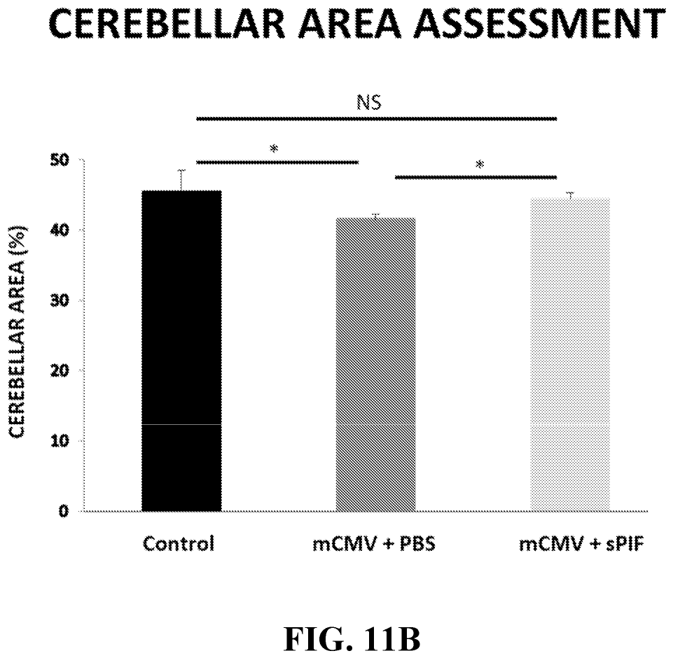

Mice were infected within 24 hours from the time of birth with 750 PFU of EGFP mCMV administered intraperitoneally (volume of administration 50 microL). sPIF was administered subcutaneously at the dosage of 0.75 mg/Kg, twice a day, from PND 2 to PND 11. Mice were sacrificed on PND 12, at least 24 hours after the last sPIF administration transcardially perfused with ice cold PBS and 4% PFA and the brains were harvested for subsequent analysis. FIG. 10. A: shows that sPIF treatment increases the weight as compared with control. The effect is significant. This is reflected also by the reduced mortality as compared to control. (B). FIG. 11A. The specific effect of sPIF on prevention of weight loss was examined showing it improved body weight as compared to control. B. The data also showed that sPIF protect the brain against infection by reducing cerebellar hypoplasia.

Therefore sPIF protects against CMV infection reducing mortality, an preventing weight loss. Considering that viral hepatitis can lead to NASH this in vivo data support such clinical potential.

### Example 4

sPIF Targets Systemic Immune Cells in NASH Model: Reduces Inflammatory CD4+Cells.

The hypercholesterolemic APO-E-model was shown to lead to vascular damage, accelerated atherosclerosis and liver steatosis. We tested whether in this model sPIF could affect the immune profile in the systemic circulation. In presence of induced inflammation the binding of FITC increased as compared with control (FITC-PIFscr).

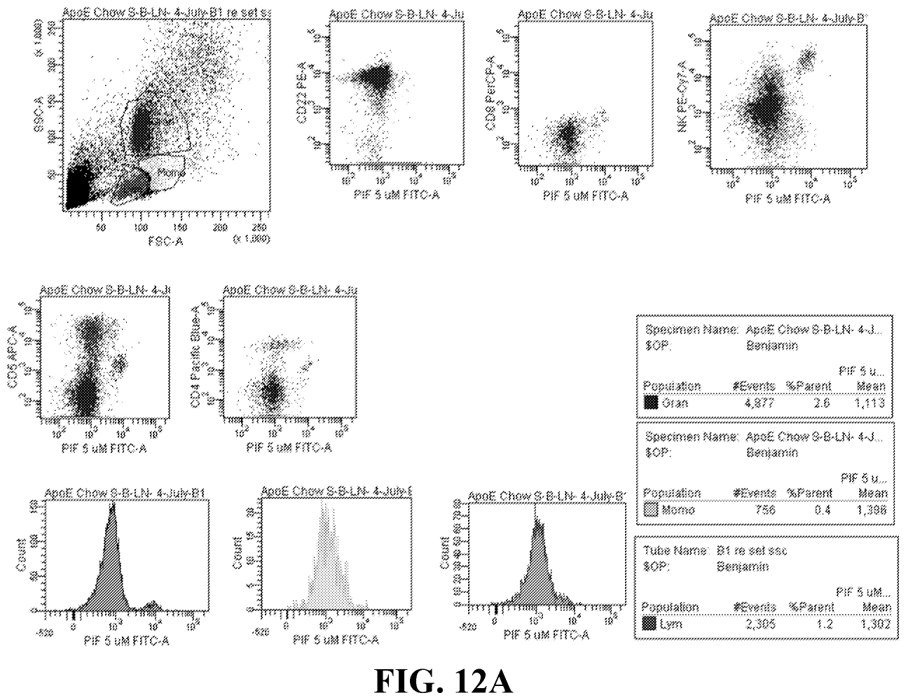

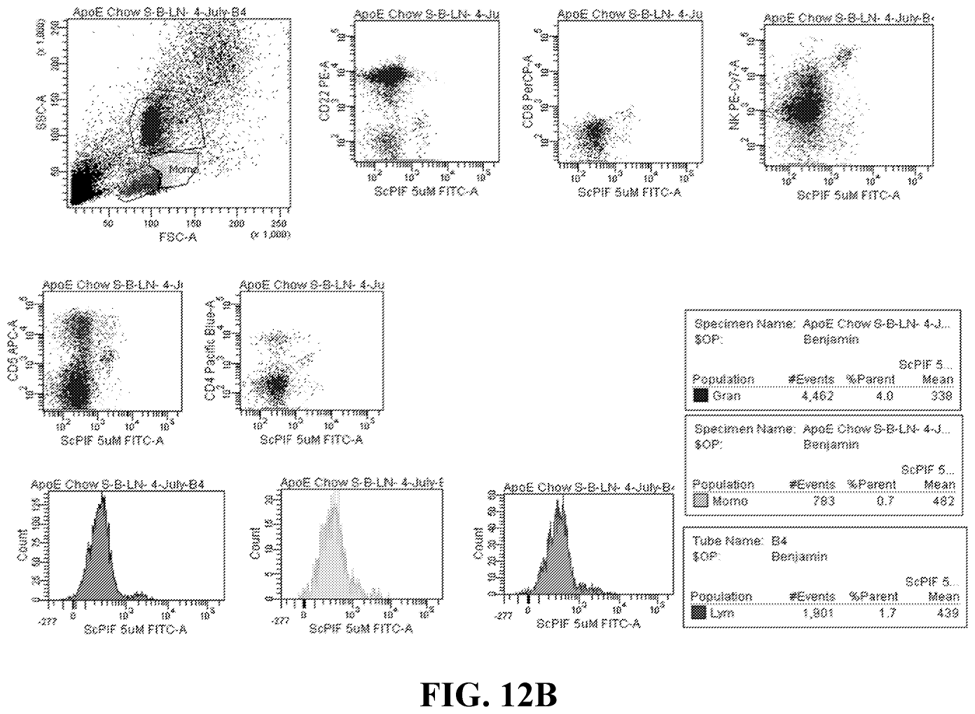

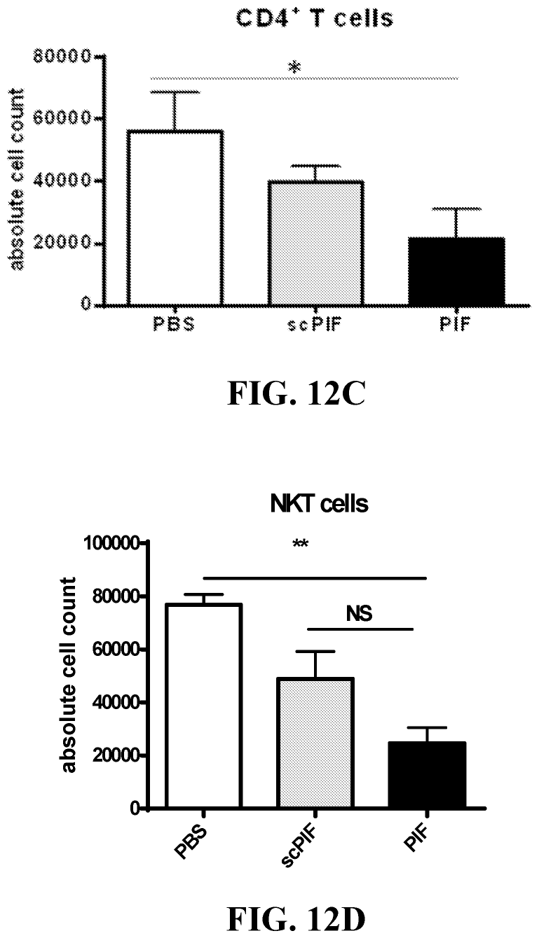

FIG. 12 A. Data showed that FITC-PIF binding to granulocytes, monocytes as well as to lymphocytes was noted. B. FITC-PIFscr-control. C. sPIF reduced systemic CD4 cells-revealing an anti-inflammatory action while not affecting NKT cells (D).

Non-alcoholic steatohepatitis (NASH) is characterized by hepatic steatosis, inflammation and fibrosis, which might progress to cirrhosis. Human NASH is associated with metabolic syndrome (MS). Currently, rodent NASH models either lack significant fibrosis or MS. ApoE−/− mice are a MS model used in cardiovascular research. We will perform a liver analysis of ApoE−/− mice treated and untreated with PIF according to the following modified protocol, which is characterized as a NASH model with significant fibrosis and MS:

Wild-type and ApoE−/− mice will be fed either with normal chow, high-fat Western diet (WD) rich in cholesterol or methionine and choline deficient diet (MCD) for about seven weeks. Body weight of animals before they are sacrificed will be observed as well as metabolic enzymes levels. ApoE−/− mice fed with WD will show a significant increase in body weight, while mice fed with MCD diet will show a decrease in body weight. Liver weight, blood analysis and liver histology will be performed on sacrificed mice to analyze signs of NASH in the liver of the animals treated and untreated with sPIF. Relative liver weight (in % of body weight) in ApoE−/− mice fed with WD should be increased in positive control group of mice, suggesting hepatomegaly, whereas it should not be altered in mice fed with MCD diet. Levels of desmosterol, hepatic cholesterol esters, hepatic triglycerides will be compared across all control and experimental animal groups.

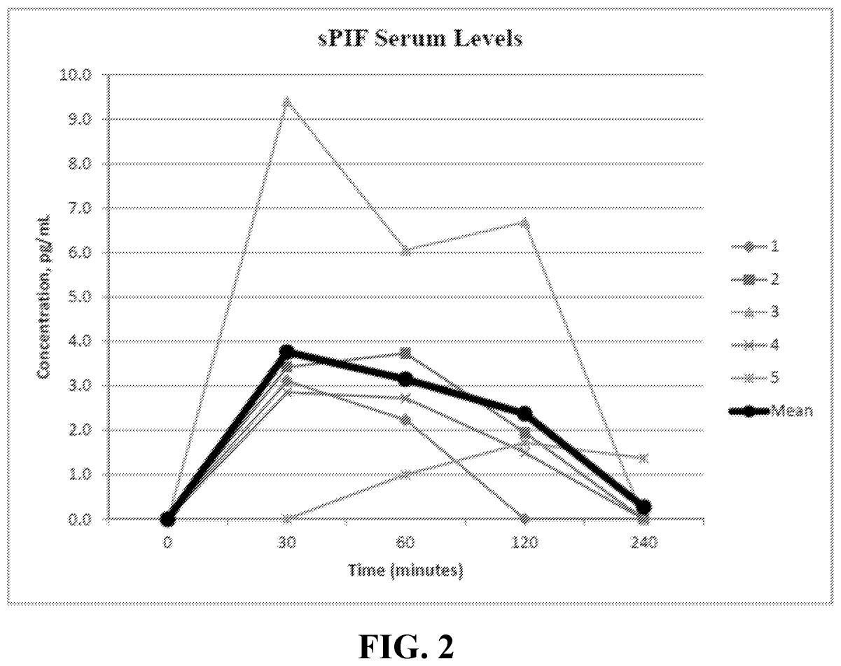

Histology of the livers of the control and experimental samples will be studied by sectioning and staining with microscopic analysis of cell features. Hepatic steatosis will be further quantified by Oil Red O staining, a histological marker for accumulation of fat in hepatocytes (FIG. 2G,H). Comparing all groups, ApoE−/− mice fed with WD should the highest amount of Oil Red O positive staining in untreated groups of animals. Compared to mice with normal chow, mice fed with MCD diet should have significantly increased Oil Red O staining, however no differences between wt and ApoE−/− mice were found. We will examine the levels of microvescular steatosis in the livers as reduction of those levels should indicate a effective biological effect of sPIF on the ApoE−/− animals fed with WD.

We will monitor the expression of the proinflammatory markers L1β, TNFα, MCP1 and Emr1 in the livers across the control and experimental groups to further characterize the NASH-like condition of livers in the animals As compared to the controls, we expect that the ApoE−/− animals fed with WD and treated with sPIF should have levels of the same genes expressed in wildtype mice either fed with WD or with MCD diet. Hepatocyte ballooning, as an important feature for the diagnosis of NASH, will be monitored in ApoE−/− mice fed WD and in mice fed with MCD diet. Interestingly, ApoE−/− mice fed normal chow and WD cells were more should be more inflated than in wt mice with respective diet or with sPIF treated ApoE−/− mice.

We will monitor fibrosis of the livers of the treated and untreated ApoE−/− animals by examining hepatic collagen deposition (hepatic hydroxyproline content, collagen mRNA), TGFβ mRNA expression and Sirius red staining. Gene expression of collagen type I (Col1a1) and profibrotic marker TGFβ should be increased in untreated ApoE^{-/-} mice fed with WD compared to wt mice fed either with WD or with MCD diet. Similarly to mRNA levels of proinflammatory markers, the expression levels of collagen and TGFβ should be highest in ApoE^{-/-} mice fed with MCD. We expect that those ApoE^{-/-} mice fed a WD and treated with sPIF should show signs of reduced collagen deposition. Study details about the mouse model are available in Schierwagen et. al, Sci Rep. 2015; 5: 12931, the contents of which are incorporated by reference in its entirety.

In conclusion, sPIF reduces the pro-inflammatory CD4+ cells thus providing systemic protection which be of relevance in protecting against NASH. It is expected that sPIF will also reduce cellular and molecular markers of NASH and fibrosis in the ApoE−/− mouse model.

### Example 5

sPIF Protects Against Streptozocin Induced Hepatotoxicity in a Primate Model.-Risk Factor for NAFLD

Various toxins can lead to liver damage which if it becomes permanent can cause NAFLD and ultimately to NASH. Whether sPIF can counteract such a toxin was examined. Streptozocin is a liver toxin where its administration lead to increase in liver enzymes. Such exposure can also lead to permanent liver injury.

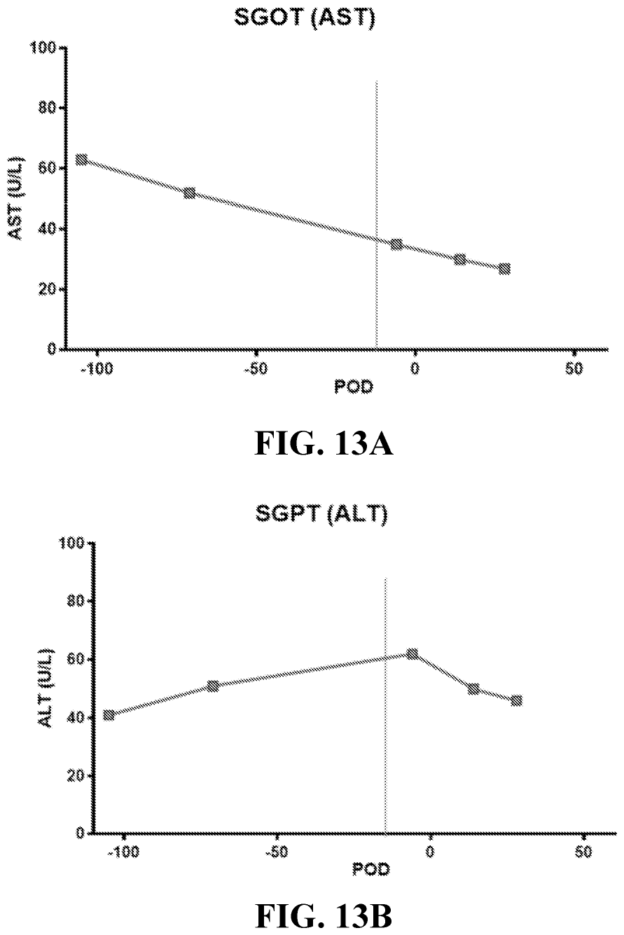

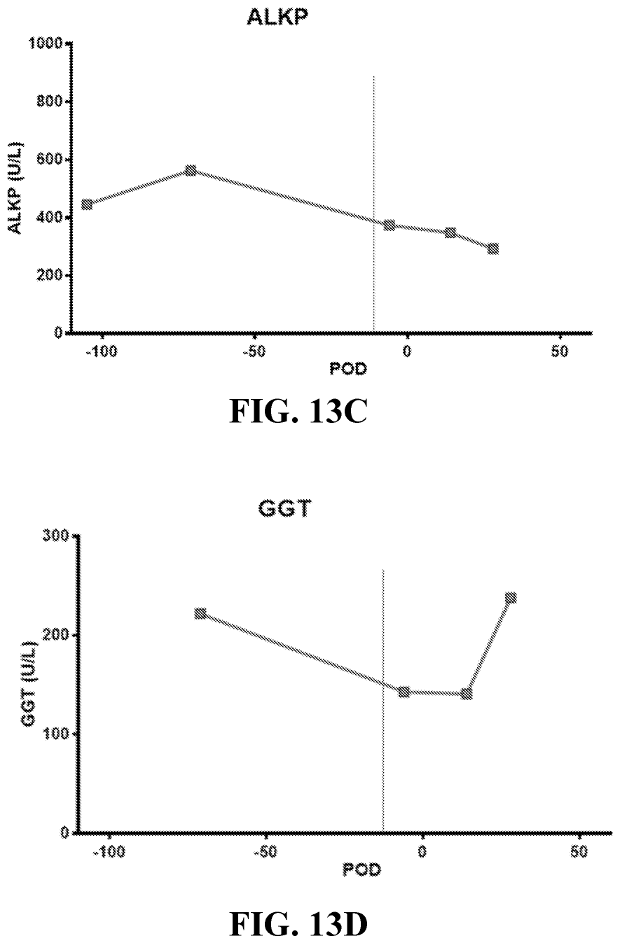

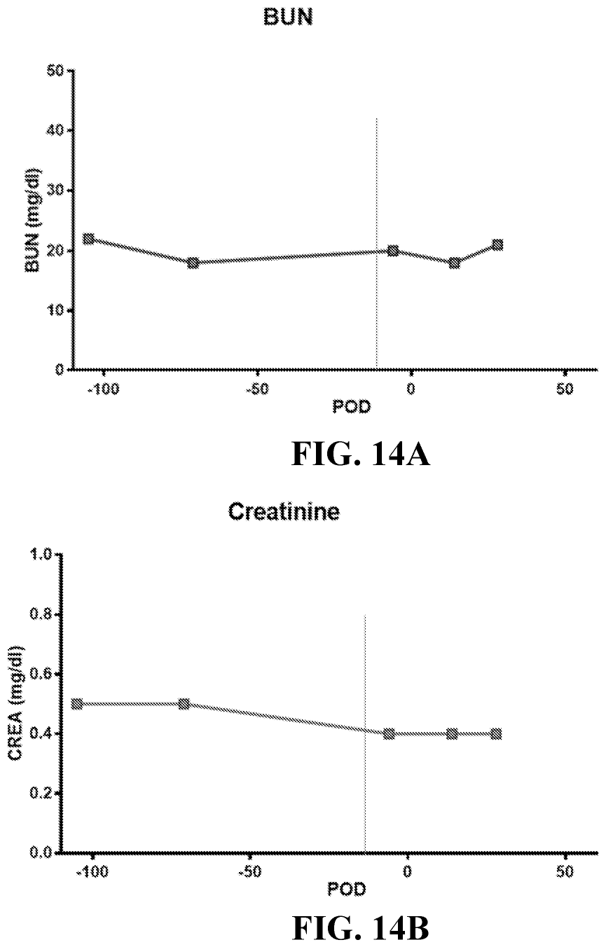

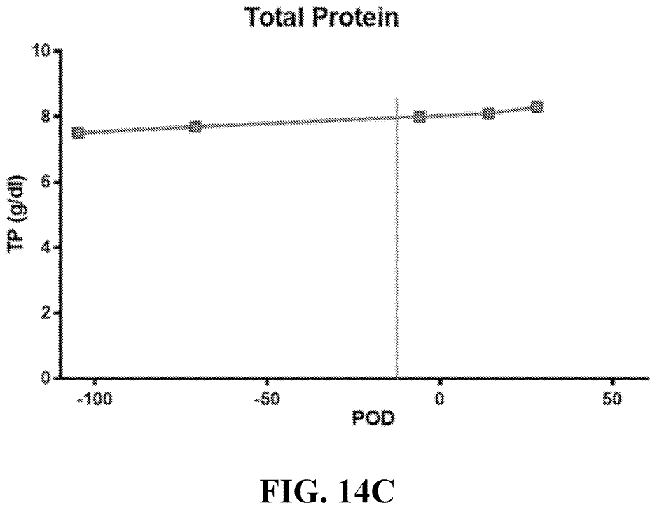

FIG. 13 The effect of Streptozocin administered to a primate 100 days before sPIF administration was tested for examining the effect on diverse liver function enzymes; ALT, AST, ALP, GGT levels. This administration leads also to diabetes by destroying the pancreatic islets. As expected all four enzymes increased following the toxic drug administration. However, as sPIF administration started at 1 mg/kg/day for 28 days caused a serial reduction in all four enzymes.
FIG. 14 In addition, sPIF also reduced creatinine thereby improving kidney function as well. Therefore, sPIF protects against drug-induced liver injury that may lead to NAFLD.

### Targets PDI/Heat Shock Proteins in the Human Liver (Attachment 4)

sPIF targets PBMCs through interaction with specific intracellular receptors involved in protecting against oxidative stress and protein misfolding namely PDI/Thioredoxin and heat shock proteins (HSPs). This was evidenced in non-obese diabetic mice where sPIF was shown to regulate pancreatic PDI and HSP proteins leading to improved diabetes control long term. To provide a strong rationale for using sPIF for treatment of liver disease beyond Phase 1 SAD and MAD data we examined the direct interaction between sPIF with normal liver tissue. Using sPIF-based affinity chromatography followed by mass spectrometry, we extracted normal liver tissue and tested the binding sites of sPIF to specific protein targets. As shown previously in human immune cells sPIF binds the same proteins in the liver. In the normal human liver tissues, the same prime targets for sPIF were identified. PDI/T, HSPs and 14-3-3, immune response/complement and cytoskeleton. In addition, several enzymes specific for the liver ethanol metabolism/oxi/redox system were identified as well. sPIF acts on the liver and the immune system by targeting the same receptors on highly specific proteins. Thus, sPIF action is both locally and systemically integrated, key factors for an effective therapeutic.

Affinity extraction Mass Spectral analysis of normal Human liver tissue lysates with PIF bound resin columns.

Previous extractions using a PIF-agarose bound column [PIF affinity chromatography column] to extract Mouse embryos and immune cell lysates derived from CD-14, 8 & 4 cell lines revealed a number of common proteins extracted for these different cell lines.

In an effort to evaluate the efficacy of this approach to the identification of PIF binding partners in Liver Disease, the same experimental approach was undertaken with three separate normal human liver tissue lysates from the same patient biopsy (Origene). It must be noted that the Lysates, while from the same patient tissue sample, are expected to exhibit heterogeneity due to the heterogeneous nature of any tissue/biopsy sample.

### PIF Affinity Extraction Protocol Using a PIF Coupled Agarose Support.

1. Dilute the protein lysate (Origene) 1:1 with binding buffer (0.2M Sodium Phosphate, pH 7.0). Mix 150 μls of lysate with 150 μls of 1×binding buffer.
2. Add 50 μls of PIF coupled Affinity Column (Biosynthesis, Lot #T5864) to the Compact Reaction Column (Part #13928 1 PK, Affymetrix, USB) (CRC) and place it in a 2.0 ml collection tube. Mix the resin gently and thoroughly before pipetting.
3. Centrifuge the column at 1250 g for 1 minute at RT.
4. Wash the column twice with 250 μls of 1×binding buffer.
5. Apply 300 μls the diluted sample to the column after sealing the bottom of the column
6. Incubate at 4° C. for 1 hour with gentle rocking motion on a rotating platform.
7. Centrifuge at 1250 g for one minute, collect the flow through and label as unbound fractions.
8. Wash the column with 400 μls of binding buffer and centrifuge at 1250 g for one minute, save the elute as wash-1.
9. Repeat Step 8 and save the elute as wash-2.
10. Apply 250 μl of elution buffer (1M glycine-HCl, pH 2.7) to the column/resin and incubate for 10 minutes with gentle rocking.
11. Place the CRC in the centrifuge collection tube collection tube containing 25 μls of neutralizing buffer (1M Tris-HCl, pH 9.0) and centrifuge at 1250 g for a minute. Label it as Eluate-1.
12. Repeat the elution with another 250 μl of elution buffer, label it as Eluate-2.
13. Keep the elutes frozen at −80 C until used.
   (Note: The binding, elution and neutralizing buffer were purchased from GE life sciences, Product #28903059, Ab Buffer Kit).

### MS Analysis of Affinity Extracted Liver Lysates

The 3 affinity extracted lysates (50 μls in 0.2 M glycine-HCl) were precipitated in 8-volume ice-cold 100% MeOH overnight at −80° C. The samples were spun to a pellet at 17,000 g for 20 min at 4° C. and the supernatant was removed. The samples were solubilized in 125 mM ammonium bicarbonate (AMBIC), 1.25 mM Ca, 0.05% Protease Max (Promega) using the Q Sonica Q800R2 sonicator. They were then reduced with 5 mM DTT and alkylated with 15 mM IAA under standard conditions. Excess IAA was quenched with an additional 5 mM DTT. Next, they were digested overnight with sequencing-grade trypsin (Promega). The next day, a 5% aliquot of each digest was acidified with 1% formic acid and heated at 95° C. for 5 min.

The peptides were separated by reversed-phase chromatography (Acclaim PepMap100 C18 column, ThermoFisher Scientific), followed by ionization with the Nanospray Flex Ion Source (ThermoFisher Scientific), and introduced into a Q Exactive mass spectrometer (ThermoFisher Scientific). Abundant species were fragmented with high-energy collision-induced dissociation (HCID). Data analysis was performed using Proteome Discoverer 2.1 (ThermoFisher Scientific) which incorporated the Sequest algorithm. The Uniprot_Hum_Compl_20160407 database was searched for human protein sequences and a reverse decoy protein database was run simultaneously for false discovery rate (FDR) determination. Sequest and X! Tandem were searched with a fragment ion mass tolerance of 0.02 Da and a parent ion tolerance of 10 PPM. Secondary analysis was performed using Scaffold (Proteome Software). Carbamidomethylation of cysteine was specified in Sequest and X! Tandem as a fixed modification. Deamidation of asparagine and glutamine, oxidation of methionine, and acetylation of the n-terminus were specified in Sequest as variable modifications. Ammonia loss of the n-terminus, deamidation of asparagine and glutamine, oxidation of methionine, and acetylation of the n-terminus were specified in X! Tandem as variable modifications.

For the 3 normal liver tissue lysates, a combined total 554 total proteins ID'ed for the 3 samples extracted. Of these, 355 are common between the three and 348 total if Keratin proteins are excluded (typical contaminants from sample handling).

### Results

Table 1 highlights the same top 25 proteins found based on total number of unique peptides identified for the protein Identified. Table 2 highlights the top 25 proteins found based on % coverage of the peptides observed for the protein Identified. Combining the identified proteins from these two tables, Table 3 highlights the 16 highest interaction proteins associated with extraction with the PIF Affinity column.

Notably, of the proteins identified, the PDI family including Calreticulin are among the highest covered proteins followed by proteins from the HSP family; notably, 78 kDa glucose-regulated protein and Endoplasmin.

Table 4 compares the proteins isolated from the Liver Tissue lysates with the proteins found to be extracted from Mouse embryo's and immune cell lines indicating that common pathways specific to PIF binding exists across diverse cell lines.

TABLE 1
Top 25 proteins identified from LC/MS/MS analysis based
on total number of unique peptides identified.

| Identified Proteins | MW | | Lysate 1 | Lysate 2 | Lysate 3 |
|---|---|---|---|---|---|
| Protein disulfide-isomerase | 57 | kDa | 268 | 359 | 235 |
| OS = Homo sapiens GN = P4HB PE = 1 SV = 3 | | | | | |
| 78 kDa glucose-regulated protein | 72 | kDa | 266 | 282 | 168 |
| OS = Homo sapiens GN = HSPA5 PE = 1 SV = 2 | | | | | |
| Protein disulfide-isomerase A4 | 73 | kDa | 194 | 217 | 185 |
| OS = Homo sapiens GN = PDIA4 PE = 1 SV = 2 | | | | | |
| Calreticulin | 48 | kDa | 176 | 199 | 192 |
| OS = Homo sapiens GN = CALR PE = 1 SV = 1 | | | | | |
| Cytosolic 10-formyltetrahydrofolate | 99 | kDa | 176 | 55 | 109 |
| dehydrogenase OS = Homo sapiens | | | | | |
| GN = ALDH1L1 PE = 1 SV = 2 | | | | | |
| Endoplasmin | 92 | kDa | 109 | 112 | 110 |
| OS = Homo sapiens GN = HSP90B1 PE = 1 SV = 1 | | | | | |
| Carbamoyl-phosphate synthase [ammonia], | 165 | kDa | 97 | 215 | 194 |
| mitochondrial | | | | | |
| OS = Homo sapiens GN = CPS1 PE = 1 SV = 2 | | | | | |
| Heat shock protein HSP 90-alpha | 85 | kDa | 90 | 63 | 98 |
| OS = Homo sapiens GN = HSP90AA1 PE = 1 SV = 5 | | | | | |
| Alpha-actinin-4 | 105 | kDa | 89 | 81 | 27 |
| OS = Homo sapiens GN = ACTN4 PE = 1 SV = 2 | | | | | |
| Transitional endoplasmic reticulum ATPase | 89 | kDa | 76 | 84 | 99 |
| OS = Homo sapiens GN = VCP PE = 1 SV = 4 | | | | | |
| Neutral alpha-glucosidase AB | 107 | kDa | 76 | 72 | 10 |
| OS = Homo sapiens GN = GANAB PE = 1 SV = 3 | | | | | |
| 14-3-3 protein epsilon | 29 | kDa | 68 | 70 | 35 |
| OS = Homo sapiens GN = YWHAE PE = 1 SV = 1 | | | | | |
| Delta-aminolevulinic acid dehydratase | 36 | kDa | 60 | 49 | 15 |
| OS = Homo sapiens GN = ALAD PE = 1 SV = 1 | | | | | |
| Glucosidase 2 subunit beta | 59 | kDa | 59 | 53 | 47 |
| OS = Homo sapiens GN = PRKCSH PE = 1 SV = 2 | | | | | |
| Protein disulfide-isomerase A3 | 57 | kDa | 57 | 74 | 42 |
| OS = Homo sapiens GN = PDIA3 PE = 1 SV = 4 | | | | | |
| Protein disulfide-isomerase A6 | 48 | kDa | 53 | 48 | 15 |
| OS = Homo sapiens GN = PDIA6 PE = 1 SV = 1 | | | | | |
| Liver carboxylesterase 1 | 63 | kDa | 51 | 99 | 13 |
| OS = Homo sapiens GN = CES1 PE = 1 SV = 2 | | | | | |
| Vimentin | 54 | kDa | 49 | 67 | 42 |
| OS = Homo sapiens GN = VIM PE = 1 SV = 4 | | | | | |
| Aldehyde dehydrogenase, mitochondrial | 56 | kDa | 45 | 73 | 56 |
| OS = Homo sapiens GN = ALDH2 PE = 1 SV = 2 | | | | | |
| Calmodulin | 17 | kDa | 45 | 27 | 23 |
| OS = Homo sapiens GN = CALM1 PE = 1 SV = 2 | | | | | |
| Alcohol dehydrogenase 1B OS = Homo sapiens | 40 | kDa | 41 | 52 | 70 |
| GN = ADH1B PE = 1 SV = 2 | | | | | |
| Fructose-bisphosphate aldolase B OS = Homo | 39 | kDa | 37 | 68 | 65 |
| sapiens GN = ALDOB PE = 1 SV = 2 | | | | | |
| Tropomyosin alpha-4 chain OS = Homo sapiens | 29 | kDa | 37 | 38 | 33 |
| GN = TPM4 PE = 1 SV = 3 | | | | | |
| Alcohol dehydrogenase 4 OS = Homo sapiens | 40 | kDa | 36 | 52 | 61 |
| GN = ADH4 PE = 1 SV = 5 | | | | | |
| High mobility group protein B1 OS = Homo sapiens | 25 | kDa | 34 | 15 | 33 |
| GN = HMGB1 PE = 1 SV = 3 | | | | | |

TABLE 2
Top 25 proteins identified from LC/MS/MS analysis based on the % Coverage
of the unique peptides observed for the protein identified.

| Identified Proteins | MW | Lysate 1 | Lysate 2 | Lysate 3 |
|---|---|---|---|---|
| 14-3-3 protein epsilon | 29 kDa | 78% | 71% | 64% |
| OS = Homo sapiens GN = YWHAE PE = 1 SV = 1 | | | | |
| Protein disulfide-isomerase | 57 kDa | 76% | 76% | 75% |
| OS = Homo sapiens GN = P4HB PE = 1 SV = 3 | | | | |
| Cytosolic 10-formyltetrahydrofolate | 99 kDa | 64% | 34% | 52% |
| dehydrogenase | | | | |
| OS = Homo sapiens GN = ALDH1L1 PE = 1 SV = 2 | | | | |
| Calreticulin | 48 kDa | 63% | 69% | 70% |
| OS = Homo sapiens GN = CALR PE = 1 SV = 1 | | | | |
| Hepatoma-derived growth factor | 27 kDa | 60% | 56% | 52% |
| OS = Homo sapiens GN = HDGF PE = 1 SV = 1 | | | | |
| Parathymosin | 12 kDa | 58% | 48% | 82% |
| OS = Homo sapiens GN = PTMS PE = 1 SV = 2 | | | | |
| Peroxiredoxin-1 | 22 kDa | 57% | 58% | 29% |
| OS = Homo sapiens GN = PRDX1 PE = 1 SV = 1 | | | | |
| 78 kDa glucose-regulated protein | 72 kDa | 55% | 51% | 48% |
| OS = Homo sapiens GN = HSPA5 PE = 1 SV = 2 | | | | |
| Alcohol dehydrogenase 4 | 40 kDa | 53% | 52% | 61% |
| OS = Homo sapiens GN = ADH4 PE = 1 SV = 5 | | | | |
| Cytochrome b-c1 complex subunit 6, | 11 kDa | 53% | 53% | 58% |
| mitochondrial OS = Homo sapiens GN = UQCRH | | | | |
| PE = 1 SV = 2 | | | | |
| Protein disulfide-isomerase A4 | 73 kDa | 52% | 52% | 51% |
| OS = Homo sapiens GN = PDIA4 PE = 1 SV = 2 | | | | |
| Transitional endoplasmic reticulum ATPase | 89 kDa | 52% | 51% | 52% |
| OS = Homo sapiens GN = VCP PE = 1 SV = 4 | | | | |
| Vimentin | 54 kDa | 50% | 49% | 52% |
| OS = Homo sapiens GN = VIM PE = 1 SV = 4 | | | | |
| Cytochrome b5 | 15 kDa | 49% | 56% | 43% |
| OS = Homo sapiens GN = CYB5A PE = 1 SV = 2 | | | | |
| Alcohol dehydrogenase 1A | 40 kDa | 48% | 53% | 54% |
| OS = Homo sapiens GN = ADH1A PE = 1 SV = 2 | | | | |
| Alpha-actinin-4 | 105 kDa | 48% | 45% | 19% |
| OS = Homo sapiens GN = ACTN4 PE = 1 SV = 2 | | | | |
| Endoplasmin | 92 kDa | 47% | 45% | 43% |
| OS = Homo sapiens GN = HSP90B1 PE = 1 SV = 1 | | | | |
| Tropomyosin alpha-4 chain | 29 kDa | 47% | 48% | 40% |
| OS = Homo sapiens GN = TPM4 PE = 1 SV = 3 | | | | |
| Alcohol dehydrogenase 1B | 40 kDa | 47% | 54% | 66% |
| OS = Homo sapiens GN = ADH1B PE = 1 SV = 2 | | | | |
| Delta-aminolevulinic acid dehydratase | 36 kDa | 47% | 49% | 25% |
| OS = Homo sapiens GN = ALAD PE = 1 SV = 1 | | | | |
| Calmodulin | 17 kDa | 46% | 46% | 46% |
| OS = Homo sapiens GN = CALM1 PE = 1 SV = 2 | | | | |
| Membrane-associated progesterone receptor | 22 kDa | 46% | 46% | 46% |
| component 1 | | | | |
| OS = Homo sapiens GN = PGRMC1 PE = 1 SV = 3 | | | | |
| Protein disulfide-isomerase A3 | 57 kDa | 45% | 44% | 42% |
| OS = Homo sapiens GN = PDIA3 PE = 1 SV = 4 | | | | |
| Enoyl-CoA hydratase domain-containing protein | 31 kDa | 45% | 42% | 31% |
| 2, mitochondrial | | | | |
| OS = Homo sapiens GN = ECHDC2 PE = 1 SV = 2 | | | | |
| Activated RNA polymerase II transcriptional | 14 kDa | 45% | 44% | 34% |
| coactivator p15 | | | | |
| OS = Homo sapiens GN = SUB1 PE = 1 SV = 3 | | | | |

TABLE 3
16 Common proteins from top 25 list in Tables 1 & 2

| Identified Proteins | MW |
|---|---|
| 14-3-3 protein epsilon | 29 kDa |
| 78 kDa glucose-regulated protein | 72 kDa |
| Alcohol dehydrogenase 1B | 40 kDa |
| Alcohol dehydrogenase 4 | 40 kDa |
| Alpha-actinin-4 | 105 kDa |
| Calmodulin | 17 kDa |
| Calreticulin | 48 kDa |
| Cytosolic 10-formyltetrahydrofolate dehydrogenase | 99 kDa |
| Delta-aminolevulinic acid dehydratase | 36 kDa |
| Endoplasmin | 92 kDa |
| Protein disulfide-isomerase A3 | 57 kDa |
| Protein disulfide-isomerase A4 | 73 kDa |
| Protein disulfide-isomerase | 57 kDa |
| Transitional endoplasmic reticulum ATPase | 89 kDa |
| Tropomyosin alpha-4 chain | 29 kDa |
| Vimentin | 54 kDa |

TABLE 4
Common Proteins extracted found in from Liver, Mouse embryo and immune
cell lysates. Yellow highlights protein common to all 5 lysates.

| | Liver | Mouse | | | |
|---|---|---|---|---|---|
| Protein ID | Lysate | embryo | CD-14 | CD-8 | CD-4 |
| 14-3-3 protein beta/alpha | X | | | | |
| 14-3-3 protein epsilon | X | X | | | |
| 14-3-3 protein eta | X | | | | |
| 14-3-3 protein gamma | X | X | | | |
| 14-3-3 protein theta | X | | | | |
| 14-3-3 protein zeta/delta | X | X | X | X | X |
| 40S ribosomal protein S3a | X | X | | | |
| 78 kDa glucose-regulated protein (HSP70 Prot 5) | X | X | | | |
| Acidic leucine-rich nuclear phosphoprotein 32 | X | | X | X | X |
| family Member A | | | | | |
| Actin, alpha skeletal muscle | X | X | | | |
| Alpha-actinin-1 | X | X | | | |
| Annexin A2 | X | X | | | |
| ATP synthase subunit beta, mitochondrial | X | X | | | |
| Calmodulin | X | | X | X | X |
| Calreticulin | X | X | X | X | X |
| Cofilin-1 | X | | X | X | X |
| Cytochrome P450 26A1 | X | X | | | |
| D-3-phosphoglycerate dehydrogenase | X | X | | | |
| Eef1a1 | X | X | | | |
| Eef1b | X | X | | | |
| Eef1d | X | X | | | |
| Elongation factor 1-alpha 1 | X | X | | | |
| Endoplasmin | X | X | | | |
| Eukaryotic initiation factor 4A-I | X | X | | | |
| Glucosidase 2 subunit beta | X | | | | |
| Hepatoma-derived growth factor | X | | X | X | X |
| Histone H2A type 1-B/E | X | | | | |
| HSP 90-alpha | X | X | X | X | X |
| Iso 2Glutamate decarbo- 1 | X | X | | | |
| Isoform 2 of Heat shock protein HSP 90-alpha | X | | X | X | X |
| Isoform 2 of Protein SET | X | X | | | |
| Isoform 2 of Tropomyosin alpha-3 chain | X | X | | | |
| Isoform 2 of Tropomyosin alpha-4 chain | X | X | | | |
| Isoform 2 of Tropomyosin beta chain | X | X | | | |
| Isoform C of Lamin-A/C | X | X | | | |
| IsoLComplement C3 | X | X | | | |
| Liver carboxylesterase N | X | X | | | |
| Nuclease-sensitive element-binding protein | X | | X | X | |
| 1 (Fragment) | | | | | |
| Nucleolin | X | X | X | X | X |
| PRDX4 | X | X | | | |
| Protein disulfide-isomerase | X | X | | | |
| Protein disulfide-isomerase A4 | X | X | | | |
| Protein disulfide-isomerase A6-like | X | X | | | |
| Serine/arginine-rich-splicing factor 1 | X | | X | X | X |
| Serine/arginine-rich-splicing factor 2 | X | | X | | X |
| Thioredoxin | X | X | | | |
| Transitional endoplasmic reticulum ATPase | X | X | | | |
| Tropomyosin alpha-4 chain | X | X | | | |

### Example 6

### NASH (Prophetic Clinical Study)

Evidence Based Rationale for Using sPIF in Nash—Fibrosis

PIF has seminal role in pregnancy biology and possible therapeutic application. sPIF is involved in immune regulation which translates to the treatment of autoimmune diseases and transplantation. The relevant preclinical models addressing efficacy in fibrosis management support this therapeutic potential. Pre-Clinical toxicological and toxicokinetic studies (rodents/canine) demonstrated high safety. Phase I trial in patients with autoimmune hepatitis (AIH) demonstrated safety and tolerability.

Pre-Clinical sPIF Effect on Metabolic/Liver Pathology-Involved in NASH-Fibrosis

Several relevant preclinical studies provide the basis for sPIFs therapeutic potential in NASH induced liver fibrosis. sPIF's addresses both local and systemic etiological factors associated with the disease.

### 1.2.7 Diabetes:

The etiology of NASH-fibrosis is due to metabolic syndrome where diabetes plays a prominent role. In a diabetic model (non-obese NOD mice), short term, low dose sPIF administration led to prevention of acute diabetes development evidenced by normalized glucose levels and normal response to glucose tolerance test.(19) The efficacy of short term sPIF administration was also demonstrated by preventing spontaneous diabetes development up to six months. Insulin expression was preserved in the islets, islet architecture was maintained, and locally the oxidative stress and protein misfolding was reduced by regulating local PDI/T and HSPs proteins. In addition, regulation of the systemic immunity by sPIF of Th1/Th2 cytokines was also observed. In conclusion, sPIF mitigates diabetes—a leading factor in NASH-fibrosis.

### High Circulating Lipids Deposits in the Liver

sPIF effect was tested APOE-E mice (NASH model). APOE-E mice fed a high fat diet develop advanced vascular inflammation and severe systemic inflammation.(20) Administration of sPIF prevented the development of accelerated atherosclerosis. Despite the high circulating lipids, sPIF prevented macrophage-induced fat deposit. Importantly, the protective effect was produced without affecting the high circulating lipids. This indicates that improved metabolic milieu can be achieved by mitigating and not reducing the high circulating lipids present in NASH patients who frequently struggle with diet and the hepatotoxic side effects of statins which limit their use. sPIF regulates macrophage function shifting them from M1 inflammatory to M2 regulatory.(21) Macrophages play an important role in liver inflammation thus sPIF could exert as similar protective effect in the liver. (22) sPIF is not immune suppressive since it does not block the oxidative burst of neutrophils required for anti-pathogen action.(20, 22-25) sPIF protective effect is dependent on the insulin degrading enzyme where sPIF regulates insulin metabolism through direct action. sPIF's protective effects in the NASH model has demonstrated reduction of circulating pro-inflammatory INF-g levels. Thus, sPIF prevents high lipid induced local and systemic inflammation without changing the high circulating lipids levels—that frequently are difficult to control in patients with NASH-fibrosis.

### Liver Inflammation-Drive NASH-Fibrosis

Four levels of data support sPIF's protection of the liver.

1. sPIF protects against semi-allogenic and totally allogenic bone marrow transplant (BMT) induced graft vs host disease (GVHD), where short term, low dose sPIF prevented development of GVHD for up to four months post-therapy.(21, 22, 26) sPIF also reverses GvHD, and promotes syngeneic bone marrow transplantation. Evidence of lack of immune suppression was shown by preservation of graft vs leukemia effect while reducing GVHD. The main target in GVHD is the liver where severe inflammation develops. Locally, oxidative stress iNOS, a hepatotoxic inducer, was reduced by sPIF treatment. In addition, proinflammatory cytokines/chemokines and their receptors expression was reduced. Skin and colon ulceration was prevented—common co-morbidity in NASH-fibrosis. This is coupled with a decrease in circulating IL1a and IL17 levels prime proinflammatory cytokines.

2. NASH-fibrosis may have a viral etiology including CMV infection. sPIF administration protected against both spleen and liver inflammation almost doubling the size of the sPIF treated rats—restoring their weight. Thus, sPIF could protect against virally induced liver inflammation.

3. In analogy to that reported on sPIF receptors in human immune cells, the binding sites in the liver are involved in oxidative stress and protein misfolding (PDI/T, HSPs) as well as aldehyde dehydrogenase-related to mitochondrial function. (24, 27) Thus, it is plausible that sPIF will exert a similar direct effect on the NASH-fibrosis patients' liver.

4. The etiology of NASH-fibrosis is not always clear. The effect of sPIF was tested on primary human hepatocytes infected with hepatitis C infection. sPIF reduced prime inflammation markers in a dose dependent manner. They include CXCL10, which is shown to promote liver fibrosis, by promoting NK cells activation.(28) In contrast, sPIF blocks human NK cells cytotoxicity.(29) CCL5 (RANTES), which plays a key role in patients with liver fibrosis, was reduced. A CCL5 antagonist used in a murine model showed reduction in fibrosis. (30) sPIF also reduced IL28 expression where homozygosity is associated with liver inflammation in hepatitis C patients.(31) RSAD2 protein over activation is associated with unfavorable hepatitis C outcome. sPIF reduced RSAD2. (32) Thus, sPIF not only binds to specific receptors in the human liver, but also reduces virally-induced liver inflammation. Overall both in vitro, human and in vivo data support liver protection against various inflammatory and pathogen induced damage leading to NASH-fibrosis.

### Fibrosis—Key to NASH Progression

sPIF can reverse fibrosis in vivo (dystrophin deficient mouse model). sPIF reversed CPK levels to baseline, a prime marker of fibrosis. In addition, sPIF administered for two weeks also reverses advanced systemic fibrosis by reducing collagen formation and leading to muscle repair at five weeks post-therapy.(33) 2. sPIF can lead to muscle differentiation was examined. sPIF treatment led to asymmetric human muscle differentiation, thus creating a reservoir of future muscle formation-instead of fibrosis. Thus, current evidence supports the view that sPIF could have similar protective properties in NASH-fibrosis not only slowing/arresting fibrosis progression, but might also lead to reduction in fibrosis that already is present.

### Clinical Data: Autoimmune Hepatitis (AIH) and Liver Fibrosis.

The HED equivalents gave a safety factor of (21-32) and demonstrated that sPIF is cleared from the circulation within four hours even at the highest dose administered even after two-week daily administration (4000 times above the therapeutic dose). Therefore, sPIF has a high safety margin. Despite the short circulating half-life due to the receptor dependent effect, sPIF's biological effect is long acting in a cascading manner. This is evidenced up to six months post two weeks administration.(35)

AIH associated liver inflammation of unknown etiology leads to progressive liver inflammation. By the time of diagnosis, ˜30% have progressed to liver fibrosis. Phase 1 placebo controlled, randomized, human, single ascending and multiple ascending dose trial in patients with normal and abnormal liver function demonstrated high safety and tolerability. Data demonstrated that there was no drug-to-drug interaction or anti-PIF antibody formed.

### 1.2.8 Single Ascending Dose

Liver function tests (LFT): ALT and AST decreased to normal levels in two patients and the effect was maintained until 8 days. A transient improvement in ALP was seen in five patients. One patient's ALP levels reached a normal range. In patients with normal liver function, sPIF effect was minimal. In patients treated with low doses of sPIF (0.1 and 0.5 mg/kg doses), circulating sPIF levels were low, however, there was improvement in both ALT and AST values.

Pharmacokinetics: sPIF demonstrated dose-proportional changes in circulating levels of the drug range tested (0.1, 0.5 and 1.0 mg/kg). sPIF plasma level in five patients were detectable ≥1 ng/ml. Levels peaked between 30 and 60 minutes after the subcutaneous injection. The increases in Cmax values were higher than dose proportional. The maximal blood level was 9.4 ng/mL. The 0.1 mg/kg sPIF dose was <1 ng/ml, below detection limit. The t1/2 of sPIF following administration was 91 minutes. Similarly, volume of distribution was 22 L using a one compartment model assumption. sPIF was not detected in the plasma at four hours after injection.

Cytokines (pg/mL): Pre-dose was compared to post-24 hours. Twenty-four hours post-injection, seven out of the twelve patients with normal or elevated ALT levels, showed a clear directional change. High concordance for trend either increase or decrease (5 cases) in Th2 (IL 10, IL4) and Th1 cytokines IL8 and IL1b and IFNγ) levels were noted one day after sPIF administration.

### 1.2.9 Multiple Ascending Dose

LFTs: Four sPIF treated patients showed improvement in LFT values from abnormal LFTs ranges. Patients with normal LFTs maintained normal range enzyme levels. Though sPIF dosing was 5 days, in some cases, improvement lasted one week and up to day 29 of the study. In two patients, ALT levels returned to the normal range. In one patient, AST and ALP levels reached the normal range. In patients with normal ALT/AST levels sPIF reduced ALP to normal range in two patients and GTT levels two-fold in one patient, reflecting an integrated effect on the liver. Overall, ten out of twelve patients showed improvement in at least one and up to four of their liver enzymes.

Pharmacokinetics: sPIF levels in all cases even at the 1 mg/kg dose were below <1 ng/ml indicating that sPIF clearance is rapid.

Cytokines (pg/mL): Cytokines were measured serially until Day 8. In six patients, although baseline IL8 levels were different, sPIF regulated this inflammatory cytokine. IL8 is an important AIH marker. In addition, in four patients, a similar trend on Th1 (IL1b, IL17a) cytokine levels was noted.

### Primary Objective of Prophetic Clinical Trial

Determine the sPIF dose that reduces liver fibrosis score from F3 to F2 as assessed by Fibroscan and reduce liver stiffness >15% from baseline as assessed by MRE at 12 and 24 weeks.

### Secondary Objective

Determine the sPIF dose that reduces elevated ALT/AST levels to the normal range at 12 weeks.

### Exploratory Assessments, at 12 Weeks

Effect on ALP/GGT
Effect on CK-18, Pro-C3
Effect on lipid profile, HbA1C
Effect on systemic immunity, cytokines and PBMC genes
Effect sPIF in plasma and anti-PIF antibody

TABLE 1
Objectives and Endpoints
Dose comparison: Induction/Maintenance Phases

| Primary Objective | Endpoint(s) for primary objective(s) |
|---|---|
| Determine sPIF 0.5 mg or 1 mg/kg dose | Fibroscan shows reduced F3 to F2 |
| administered up to 12 weeks that reduces | fibrosis, and MRE ≥15%, |
| fibrosis and liver stiffness | repeat at 24 weeks |
| Secondary objective | |
| Determine the 0.5 mg or 1 mg/kg, respectively | Change of ALT/AST from baseline to |
| dosing interval required to maintain ALT/AST | normal range |
| in normal range up 12 weeks | |
| Evaluate the safety and tolerability of the | . No SAE ≥ Grade 3 observed |
| increased dose and treatment duration | |
| Exploratory Objective(s) | Endpoint(s) for exploratory objective(s) |
| Analyze liver indices, PBMC genomics and | Improve liver indices and systemic |
| cytokine levels | immune response |
| Determine effect on circulating lipids/HbA1C levels | Changes in lipid profile/HbA1C |
| Measure effect on CK-18 and Pro-C3 levels | Improvement in circulating fibrosis markers |
| Determine anti-sPIF antibody titer | Anti-sPIF Antibody titer detection |
| Assess plasma sPIF level | sPIF clearance from circulation |

TABLE 2
Dose escalation: Induction/Maintenance Phases

| Primary Objective | Endpoint(s) for primary objective(s) |
|---|---|
| Determine effect of sPIF 0.5 mg dose | Fibroscan F3 to F2, fibrosis reduction |
| increments administered up to 12 weeks on | and MRE ≥15%, reduced stiffness, |
| fibrosis and liver stiffness | repeat at 24 weeks |
| Secondary objective | |
| Determine the 0.5 mg dose increments | Change of ALT/AST from baseline to |
| required to maintain ALT/AST levels in the | normal range. |
| normal range up 12 weeks | |
| Evaluate the safety and tolerability of the | No SAE ≥ Grade 3 observed |
| increased dose and treatment duration | |
| Exploratory Objective(s) | Endpoint(s) for exploratory objective(s) |
| Analyze liver indices, PBMC genomics and | Improve liver indices and systemic |
| cytokine levels analysis | immune response |
| Determine effect on circulating lipids/HbA1C levels | Changes in lipid profile and HbA1C |
| Measure effect on CK-18 and Pro-C3 levels | Improvement in circulating fibrosis markers |
| Determine anti-sPIF antibody titer up to 12 weeks | Anti-sPIF Antibody titer detection |
| Assess plasma sPIF level up to 12 weeks | sPIF clearance from circulation |

### 2. Population

The study population will consist of male and female patients ages 18 to 75 years with NASH induced liver fibrosis with compensated chronic liver disease currently under standard of care. Patients will be recruited from the Center for Liver Diseases' clinical practice at the University of Miami Hospital (UMH) and the University of Miami Hospital and Clinics (UMHC) at the start of the study. Additional U.S. centers (approximately 5-10) and outside the U.S. may be added once effective dose is determined within a cohort. The goal is to screen/enroll approximately a total of 60 patients.

### 2.2 Inclusion Criteria

Patients eligible for inclusion in this study must fulfill all the following criteria: Written informed consent must be obtained before any assessment is performed. Males and females aged from 18 to 75 years old
Females must be either:
Postmenopausal for greater than two years
Postmenopausal for less than two years with an FSH level greater ≥40 mlU/mL
Documented as surgically sterile (bilateral tubal ligation, bilateral oophorectomy or post-hysterectomy) at least three months prior to the screening evaluation
Or be greater than age 40, does not want more children, is currently using at least one effective method of birth control at the time of screening and agrees to using two effective methods of birth control starting with Study Day 0 and through the duration of the study
Diagnosis of liver fibrosis by Fibroscan to demonstrate F3 (8.5 to 12.5 kPA) and determine in the liver the extent of fat and degree of steatosis
MRE determining degree of liver stiffness
Elevated AST, ALT levels, above the normal range of reference laboratory
Patients having negative HbsAg and HIV, with Anti-HCV antibody but negative HCV RNA without therapy
Patients with metabolic syndrome, obesity, BMI≥30, diabetes HbA1c≥7, abnormal lipid profile LDL≥120, HDL<40 mg/dL
ALT and AST<5-fold (ULN) values with no clinical or laboratory evidence of hepatic decompensation (i.e., platelets≤100,000/mm³, total bilirubin≥1.5×ULN, prothrombin time≥1.2×ULN, albumin≤3.0 g/dL and AFP≥50 ug/L
Prednisone and/or other oral, immunosuppressive drug(s) use must be stabilized for 4 weeks prior to screening

### 2.3 Exclusion Criteria

Patients fulfilling any of the following criteria are not eligible for inclusion in this study. No additional exclusions may be applied by the Investigator, to ensure that the study population will be representative of all eligible patients.
Child-Pugh class C
Use of a mean daily dose of 80 g alcohol within one month, enzyme inducers or inhibitors, or drugs of abuse that might affect this study
Ascites, hemorrhage from portal varicose veins, uncompensated liver cirrhosis with the history of hepatic encephalopathy within the 6 months
Hepatocellular carcinoma or presence of a suspicious foci on hepatic ultrasonography at screening or liver transplantation
Pregnancy or lactation
Serious concurrent illness, renal-low <60 GFR, pulmonary-moderate COPD, neurological-severe paralysis, cardiovascular (CHF of class Ill or above; a history of MI within the past 6 months) diseases, or any cancer, autoimmune disorders or severe depression)
Any patient who is inappropriate to join clinical study as judged by participating clinicians
Bilirubin≥2.0 mg/dL, PT≥40 sec, and serum albumin<2.5 g/dL, AFP>50 ug/L, PLT≤100,000/mm³
Patient who has participated in other clinical trials within recent 3 months.
Patients who may receive chemotherapeutic or immune suppressive agents (e.g., corticosteroids, immunoglobulins and other immune- or cytokine-based therapies) during the study for any other medical condition

### 3. Treatment

### 3.2 Study Treatment

### 3.2.7 Investigational Drug

Endogenous PIF is an evolutionarily conserved peptide consisting of 15 amino acid residues (single-letter amino acid sequence: MVRIKPGSANKPSDD) that is endogenous to the circulatory system of a pregnant human and other mammalian females. sPIF INJECTION is a sterile, aqueous solution intended for subcutaneous injection.

The three-letter amino acid sequence of PIF and sPIF is: H-Met1-Val-Arg-Ile-Lys5-Pro-Gly-Ser-Ala-Asn10-Lys-Pro-Ser-Asp-Asp15-OH

The sequence contains only natural L-amino acids (except for glycine, which is optically inactive), with a C-terminal carboxylic acid group. sPIF is chemically identical to the biologically produced human native peptide. The biologic activities of sPIF and PIF are indistinguishable.

sPIF is a GMP manufactured peptide at PolyPeptide (San Diego, Calif., USA) and is bottled, filled, and sterilized at Piramal (Lexington, Ky., USA). sPIF will be reconstituted in the pharmacy from powder form (lyophilized) to a 0.5 mL total volume per syringe with lactated Ringer's solution, USP to the dose appropriate to the patient's weight at mg/kg. The purity specification for sPIF API is greater than 97%. The sPIF Active Pharmaceutical Ingredient (API) is isolated with acetate counterion (ca. 3-4 acetates per peptide molecule, limited to no more than 15% (weight % of the API). Residual water is limited to no more than 10% (weight % of the API). The sPIF is a white to off-white powder. No crystalline or polymorphic forms are known. sPIF is absent of raw materials or contacted processing materials in the manufacture that could cause exposure to bovine spongiform encephalopathy/Transmissible Spongiform Encephalopathy (BSE/TSE).

### 3.3 Treatment Arms

The study is an open label dose comparison and dose finding adaptive design. Patients will be dosed at cohorts of (n=7).

### 3.4 Patient Numbering

Once patients sign an informed consent, he or she will be assigned a screening number. The assignment of the screening number will be made in sequence upon a patient signing a consent form to participate in the clinical trial. Number sequence will be 001, 002, 003, etc.

Patients may be rescreened once as follows:
Patients who meet all eligibility criteria except for one exclusionary lab parameter may rescreen without prior sponsor approval
Patients who fail to enroll within the 30 days of screening may rescreen once without approval of the sponsor
Patients being re-screened must have all laboratory studies performed. Chest x-ray will not be done
Patients who meet the eligibility criteria will keep their original screening number.

### 3.5 Dispensing the Study Drug

The sPIF will be labeled in accordance with appropriate Federal and/or local drug dispensing laws. The labels will include, at a minimum, the following information: name, product name and strength (sPIF), bottle number (a unique identifier specific to that bottle); caution statements “Caution New Drug—Limited by Federal (or United States) Law to Investigational Use”, and the Investigator's name. Sufficient quantities of the sPIF will be supplied to the Investigator (or qualified designee, e.g., the study center pharmacist). The study drug will be shipped directly to the University of Miami Center for Liver Diseases or the University of Miami Pharmacy attention to the Principal Investigator (or its designee) using a registered courier service. Alternatively, to other participating clinical sites. sPIF should remain in powder form in a tightly capped bottle where it is stored under nitrogen. This form of storage has been documented to be stable for several years. For long-term storage, it should be stored in a −20° C. designated or its equivalent freezer. Once it is reconstituted it should be stored at 4° C. and administered within 48 hours. Until dispensed to the patients, the bottles of sPIF should be stored in a securely locked area, accessible only to authorized site personnel. To ensure the stability of sPIF and proper product identification, the drug product should be stored in the original container and will not be re-packaged into another container. The requirements of all applicable Federal and/or local drug dispensing laws will apply to the doses of sPIF dispensed by the pharmacist (or qualified designee) for administration to individual patients in the clinic at the study center. For each dose of study drug administered, the pharmacist (or qualified designee) will record the vial number in the drug accountability form.

1.5 Instructions for Prescribing and Taking Study Drug Treatment

sPIF will be stored in the hospital pharmacy at the University of Miami Center for Liver Diseases and/or the University Pharmacy at other sites at −20° C. Once a patient is consented and assigned a patient number, the patient's weight will be noted and sPIF dose will be reconstituted in lactated Ringer's solution and dispensed based on patient's weight. Reconstituted drug vials may be stored at 2-8° C. until use. sPIF will be administered by insulin syringes for injection of a single subcutaneous dose after reconstitution at 0.5 mL total volume for each injection. Subjects participating in the Induction Phase of the protocol will receive 14 doses of sPIF administered Day 1-14 by a study nurse at the University of Miami's Center for Liver Diseases or the Clinical Research Center (CRC) or the patients will receive the necessary instruction for self-administration at home.

### 3.6 Concomitant Medications

Details of all prior (within 30 days of the screening evaluation) and concomitant medication use, including all medications administered for the treatment of adverse events, will be recorded in the patient's CRF. After a patient has received their first dose of sPIF, if the patient should require treatment with a medication for an adverse event, the patient's continued participation in the study will be re-evaluated by the Investigator on an ongoing, case-by-case basis. Patients will be requested to refrain from the consumption of alcohol until after the final clinical laboratory safety tests at the follow-up evaluation is completed. The Investigator must instruct the patient to notify the study site about any new medications he/she takes after the patient is enrolled into the study. All medications, procedures and significant non-drug therapies (including physical therapy and blood transfusions) administered after the patient is enrolled into the study must be recorded in the concomitant medications/significant non-drug therapies eCRF.

### 4. Investigational Plan

### 4.2 Study Design

This is an open-label adaptive design study divided into two parts: Induction Phase and Maintenance Phase comparing baseline Fibroscan and MRE imaging with results at 12 weeks and repeated at 24 weeks. The secondary objective is the improvement of ALT/AST levels. The dose comparison will be followed as needed by dose escalation. Each consecutive cohort will contain 7 patients. Beyond the dose comparison, only after all patients in each cohort have completed the dosing, escalation to the next dose level will occur upon review and assessment of the clinical and laboratory results. Additional patients may be required to replace enrolled patients who are withdrawn from the study or decline to participate in the protocol. Patients will be recruited from the Center for Liver Diseases' clinical practice at the University of Miami Hospital (UMH) and the University of Miami Hospital and Clinics (UMHC). Additional sites in the U.S. and abroad may be added. Patients who discontinue for any reason the study in any part, may be replaced but will be advanced to the end of study visit and for the 30 days post-treatment monitoring, if possible.

### 4.3 Monitoring:

Periodic safety monitoring which includes symptom-driven physical examination, vital signs measurements, clinical laboratory testing, 12-lead ECG, and the recording of adverse events and concomitant medication intake will be determined. This will be performed at screening, prior to the first dose administration on Day 1 (to establish baseline), in each part, and then at periodic intervals (see visit tables Appendix 1 and Appendix 2) throughout the treatment phase of both parts. Serial blood sampling for safety testing will be collected:

### 5. Visit Schedule and Assessments

The study is divided into 2 parts: Induction/Maintenance Phases consisting of dose identification and maintenance.

Appendix 1 and Appendix 2 lists all the assessments and indicates with an “x” when the visits are performed.

| Screening Evaluation Day -28 and Pre-Dose | |
|---|---|
| sPIF (Day 1) Induction Phase | sPIF (Day 5, 8, 14) |
| Assessment informed consent (screening) | Assessment |
| Medical history, review of prior and | Record use of concomitant medications |
| current medication and alcohol use | |
| Inclusion criteria and confirmation that no | Medical history update |
| exclusion criteria apply | |
| Vital signs measurement (sitting blood | Vital signs measurement (sitting blood |
| pressure, pulse rate, respiration rate and | pressure, pulse rate, respiration rate and |
| oral temperature) height (cm), and body | oral temperature) height (cm), and body |
| weight (kg) | weight (kg) |
| Complete physical examination | Symptom driven physical exam as necessary |
| Fibroscan/MRE (screening) | |
| Blood Sample Collection | Serum chemistry: sodium, potassium, |
| | chloride, bicarbonate, blood urea nitrogen |
| | (BUN), creatinine, glucose |
| Hematology: complete blood count (CBC) | Hematology: complete blood count (CBC) |
| with white blood cell (WBC) differential and | with white blood cell (WBC) differential and |
| platelet count (PLT) | platelet count (PLT) |
| Serum chemistry: sodium, potassium, | Liver function tests: total bilirubin, ALT, |
| chloride, bicarbonate, blood urea nitrogen | AST, ALP, GGT total protein, albumin |
| (BUN), creatinine, glucose | |
| Liver function tests: total bilirubin, ALT AST, | Other: Amylase, creatine phosphokinase |
| ALP, GGT, total protein, albumin | (CK), magnesium, phosphorus, and uric |
| | acid (Day 14) |
| Coagulation: PT, PTT (screening) | PBMC genomics and cytokine analysis |
| Lipids: cholesterol (total, fasting), | Coagulation: PT, PTT (Day 14) |
| triglycerides (fasting), HbA1C (predose) | |
| Other: Amylase, creatine phosphokinase (CK), | |
| magnesium, phosphorus, and uric acid (screening) | |
| Alpha-fetoprotein (screening) | Plasma sPIF level (Day 14) |
| CK-18, Pro-C3 (pre-dose) | CK-18, Pro-C3 (Day 14) |
| PBMC genomics, and cytokine analysis (pre-dose) | |
| Anti-sPIF antibody (pre-dose) | |
| Urine analysis pregnancy test if applicable. | Urine analysis (macroscopic, reflex to |
| Urine analysis (macroscopic, reflex to | microscopic urinalysis if dipstick result is |
| microscopic urinalysis if dipstick result is | abnormal): appearance, blood, colour, |
| abnormal): appearance, blood, colour, glucose, | glucose, leukocyte esterase, pH, protein and |
| leukocyte esterase, pH, protein and urobilinogen | urobilinogen |
| Electrocardiogram Twelve-lead ECG (recording | |
| is made after the patient has rested in a semi- | |
| supine position for ≥5 minutes) (pre-dose) | |
| Chest x-ray PA and lateral. (screening) | |

Patients must be seen for all visits on the designated day, +/−3 days. Missed or rescheduled visits should not lead to automatic discontinuation. Patients who prematurely discontinue the study for any reason should be scheduled for a visit as soon as possible, at which time all the assessments listed for the final visit will be performed. Patients will be contacted after 30 days from the last drug administration to assess safety and their concomitant medications will be reconciled.

The following text describes the study procedures that will be completed both in the dose comparison and dose escalation study.

Maintenance Phase Days 28, 42, 56, 70 and 84
Record use of concomitant medications
Vital signs measurement (sitting blood pressure, pulse rate, respiration rate and oral
temperature) height (cm), and body weight (kg)
Medical history update/Physical exam (as needed)
Blood Sample Collection
Serum chemistry: sodium, potassium, chloride, bicarbonate, blood urea nitrogen
(BUN), creatinine, glucose (Days 28, 56, 84)
Hematology: complete blood count (CBC) with white blood cell (WBC) differential
and platelet count (PLT) (28, 56, 84
Coagulation: PT, PTT (Day 28, 56, 84)
Liver function tests: total bilirubin, alkaline phosphatase, ALT, AST, ALP, GGT total
protein, albumin
Lipids: cholesterol (total, fasting), triglycerides (fasting), HbA1C (Days, 84)
Other: Amylase, creatine phosphokinase (CK), magnesium, phosphorus, and uric
acid (Days 28, 56, 84)
PBMC genomics and serum cytokine analysis
ECG (Day 84)
CK-18, Pro-C3
Anti-sPIF antibody (Days 28, 56, 84)
Urine analysis Urine pregnancy test (Day 28, 84), if applicable. Analysis (macroscopic,
reflex to microscopic urinalysis if dipstick result is abnormal): appearance, blood, glucose,
leukocyte esterase, pH, protein and urobilinogen
Fibroscan/MRE Day 84 (12 weeks) and 24 weeks

Additional follow up as needed Additional follow-up visits will be scheduled if a patient has an ongoing clinically significant abnormality at the follow-up evaluation and/or the clinical laboratory testing performed at the follow-up evaluation subsequently reveals a clinically significant laboratory abnormality.

Clinical abnormalities or clinically significant abnormal laboratory values should be monitored periodically, as deemed prudent by the Investigator (or designee), until the abnormality(s) resolve, returns to baseline levels, or are otherwise explained.

### 5.2 Additional Analyses (Exploratory Assays)

Cytokine testing in serum collected will be collected and tested by multiplex assay. Microarray Gene Analysis of immune cells covering autoimmune inflammatory markers. Assessment of anti-drug (sPIF) antibodies in serum at different intervals will be tested. A commercial M30-Apoptosense ELISA Kit (PEVIVA, Sweden) will be used for CK-18 determination and for Pro-C3 PRO-C3 (true collagen type Ill formation) levels and ELISA method will be used.(36)

### 5.2.7 Clinical Laboratory Testing

Local laboratory or commercial laboratories will be used as per need.

### 5.2.8 Blood Sampling for Pharmacokinetic/Pharmacodynamic Analyses

The testing for sPIF levels in the circulation will be carried out using sensitive liquid chromatography and mass spectroscopy (LC-MS/MS) at Covance.

The blood serum/plasma samples for long-term storage will be collected for possible future testing. After this study, these samples may be retained in storage for a period up to 15 years. The packaging, labeling and storage of these samples at BioIncept designated laboratory.

### Example 7

Recombinant sPIF Attachment 6 (Protocol)

Currently sPIF is being manufactured by solid synthesis. Whether sPIF can be expressed in a cell was tested. The data showed that sPIF is possible to be manufactured by recombinant technology.

A gene coding GB1 domain and PIF-15 (MVRIKPGSANKPSDD) peptide was chemically synthesized by Integrated DNA Technologies. The full sequence was amplified by PCR and digested by BgIII/Xhol. The digested DNA was cloned to pRSFDuet-1 DNA vector precut by BamHI/Xhol. The resulted plasmid (pMB1705) was verified by DNA sequencing.

pMB1705 plasmid was transformed to competent BL21 (DE3) cells and cultured overnight at 37° C. on LB-agar plate containing kanamycin.

Single bacterial colony emerging from the plate was isolated and cultured in terrific broth medium, containing kanamycin. When OD600 achieved 0.8, IPTG was added to 1 mmol/L to induce the expression at 25° C. for 16-24 hours. The culture was harvested by centrifuge. The cell pellet was suspended in PBS buffer, pH7.4 and sonicated until the solution was no longer viscous. The His6-GB1-TEV-PIF fusion protein was expressed in the soluble form as determined by 4-12% SDS-PAGE gel.

The expression clone was inoculate and grew in 2 liters terrific broth. IPTG was added to 1 mmol/L when OD600 achieved 0.8. Cells were harvested by centrifuge, suspended in PBS buffer, pH7.4, and sonicated. After centrifuge, the cell lysate was loaded to Cobalt resin. After extensive wash with PBS, the bound protein was eluted by PBS, containing 150 mM imidazole and dialyzed against 20 mM Tris-HCl, 150 mM NaCl, pH7.5. The purified protein was assessed 12% SDS-PAGE

Thirty milligrams purified protein was incubated with 1 mg TEV protease to release PIF peptide. After overnight incubation, the released peptide was purified by protein concentrator (MWCO ˜3,000 Da). The purified peptide was analyzed by MALDI-TOF. The molecular weight of the peptide is 1613.19 Da

1. A method of treating and/or preventing fibrosis in a subject in need thereof, the method comprising: administering to the subject a pharmaceutical composition comprising: (i) a therapeutically effective amount of PIF peptide, compositions thereof, mimetics thereof, pharmaceutically acceptable salts thereof, or combinations of any of the foregoing; and (ii) one or a plurality of pharmaceutically acceptable carriers.

2. The method of claim 1, wherein the therapeutically effective amount of PIF peptide is from about 0.01 μg per milliliter of volume to about 10 mg per milliliter of volume.

3. (canceled)

4. The method of claim 1, wherein the PIF peptide comprises MVRIK (SEQ ID NO: 18) or a pharmaceutically acceptable salt thereof.

5-7. (canceled)

8. The method of claim 1, wherein the method further comprises administering a therapeutically effective amount of an anti-inflammatory compound at least one hour prior to, simultaneously with, or subsequent to administering the pharmaceutical composition.

9. The method of claim 8, wherein the anti-inflammatory is free of a steroid.

10. The method of claim 1 further comprising a step of administering a therapeutically effective amount of a PIF peptide, compositions thereof, mimetics thereof, pharmaceutically acceptable salts thereof, or combinations thereof, once, twice, or three times in a 30 day period.

11. The method of claim 1, wherein the therapeutically effective amount of a PIF peptide is the only therapeutic compound administered to the subject.

12. The method of claim 1, wherein the pharmaceutical composition is administered intravenously, intramuscularly, topically intradermally, transmucosally, subcutaneously, sublingually, orally, intravaginally, intraocularly, intranasally, intrarectally, gastrointesinally, intraductally, inthecally, subdurally, exradurally, intraventricularly, intraarticuarly, intraperitoneally, or into the pleural cavity.

13-24. (canceled)

25. A method of treating and/or preventing non-alcoholic steatohepatitis (NASH) in a subject in need thereof, the method comprising: administering to the subject a pharmaceutical composition comprising: (i) a therapeutically effective amount of PIF peptide, compositions thereof, mimetics thereof, pharmaceutically acceptable salts thereof, or combinations of any of the foregoing; and (ii) one or a plurality of pharmaceutically acceptable carriers.

26. The method of claim 25, the therapeutically effective amount is from about 0.01 μg per milliliter of volume to about 10 mg per milliliter of volume.

27. (canceled)

28. The method of claim 25, wherein the PIF peptide comprises MVRIK (SEQ ID NO: 18) or a pharmaceutically acceptable salt thereof.

29-31. (canceled)

32. The method of claim 25, wherein the method further comprises administering a therapeutically effective amount of an anti-inflammatory compound at least one hour prior to, simultaneously with, or subsequent to administering the pharmaceutical composition.

33. The method of claim 32, wherein the anti-inflammatory is free of a steroid.

34. The method of claim 25 further comprising a step of administering a therapeutically effective amount of a PIF peptide, compositions thereof, mimetics thereof, pharmaceutically acceptable salts thereof, or combinations thereof, once, twice, or three times in a 30 day period.

35. The method of claim 25, wherein the therapeutically effective amount of a PIF peptide is the only therapeutic compound administered to the subject.

36. The method of claim 25, wherein the pharmaceutical composition is administered intravenously, intramuscularly, topically intradermally, transmucosally, subcutaneously, sublingually, orally, intravaginally, intraocularly, intranasally, intrarectally, gastrointesinally, intraductally, inthecall, subdurally, exradurally, intraventricularly, intraarticuarly, intraperitoneally, or into the pleural cavity.

37-48. (canceled)

49. A method of treating and/or preventing viral infection in a subject in need thereof, the method comprising: administering to the subject a pharmaceutical composition comprising: (i) a therapeutically effective amount of PIF peptide, compositions thereof, mimetics thereof, pharmaceutically acceptable salts thereof, or combinations of any of the foregoing; and (ii) one or a plurality of pharmaceutically acceptable carriers.

50. The method of claim 49, the therapeutically effective amount is from about 0.01 μg per milliliter of volume to about 10 mg per milliliter of volume.

51. (canceled)

52. The method of claim 49, wherein the PIF peptide comprises MVRIK (SEQ ID NO: 18) or a pharmaceutically acceptable salt thereof.

53-55. (canceled)

56. The method of claim 49, wherein the method further comprises administering a therapeutically effective amount of an anti-inflammatory compound at least one hour prior to, simultaneously with, or subsequent to administering the pharmaceutical composition.

57. The method of claim 49, wherein the viral infection is a CMV infection or Hepatitis C infection.

58-65. (canceled)

66. The method of claim 25, wherein the administration of the pharmaceutical composition further treats and/or prevents NASH-induced fibrosis in the subject simultaneously to treating/and or preventing NASH.
